(19) United States
(12) Patent Application Publication
(10) Pub. No.: US 2016/0047000 A1
(43) Pub. Date: Feb. 18, 2016

(54) METHODS AND SYSTEMS FOR TREATMENT OF OVARIAN CANCER

(71) Applicants: GENERAL HOSPITAL CORPORATION, Boston, MA (US); DANA-FARBER CANCER INSTITUTE, INC., Boston, MA (US)

(72) Inventors: Michael BIRRER, Walpole, MA (US); Giovanni Luigi PARMIGIANI, Brookline, MA (US); Markus RIESTER, Jamaica Plain, MA (US); Wei WEI, Melrose, MA (US)

(21) Appl. No.: 14/778,832

(22) PCT Filed: Mar. 20, 2014
(86) PCT No.: PCT/US14/31295

§ 371 Date: Sep. 21, 2015

Related U.S. Application Data
(60) Provisional application No. 61/803,919, filed on Mar. 21, 2013.

(51) Int. Cl. C12Q 1/68; G01N 33/574

(52) U.S. Cl. CPC C12Q 1/6886; G01N 33/57449; C12Q 2600/158; C12Q 2600/106; G01N 2800/52

(57) ABSTRACT

As described herein, the inventors have identified gene signatures which permit the identification of patients who will benefit from (e.g. have optimal outcomes) cytoreductive surgery as treatment for ovarian cancer. Accordingly, provided herein are methods of treatment, assays, and systems relating to ovarian cancer and the administration of cytoreductive surgery. In one aspect, the technology described herein relates to a method of treatment comprising, detecting, in a sample obtained from a subject in need of treatment for ovarian cancer, the level of activation of at least one pathway, and administering cytoreductive surgery to the subject if the level of activation is not increased relative to a reference level.

### CROSS-REFERENCE TO RELATED APPLICATIONS

This application claims benefit under 35 U.S.C. §119(e) of U.S. Provisional Application No. 61/803,919 filed Mar. 21, 2013, the contents of which are incorporated herein by reference in their entirety.

### GOVERNMENT SUPPORT

The present application was made with Government support under Grant Numbers 1RC4CA156551-01 1R01CA142832-01, and 5U01CA152990 awarded by the National Institutes of Health and DBI-1053486 awarded by the National Science Foundation. The Government of the United States has certain rights in the invention.

### SEQUENCE LISTING

The instant application contains a Sequence Listing which has been submitted electronically in ASCII format and is hereby incorporated by reference in its entirety. Said ASCII copy, created on Mar. 20, 2014, is named 030258-077301-PCT_SL.txt and is 94,113 bytes in size.

### TECHNICAL FIELD

The invention relates to methods and systems of treating and prognosing ovarian cancer.

### BACKGROUND

Ovarian cancer is the most lethal gynecologic malignancy, causing over 15,000 deaths per year in the United States alone. While significant improvements have been made in the median survival of women with advanced stage ovarian cancer, overall survival has essentially not changed during the last decades. One treatment option for ovarian cancer involves invasive cytoreductive surgery to remove as much tumor tissue as possible. However, a certain percentage of patients will have cancers which cannot adequately be removed by such surgeries. With current technologies available in the clinic, it is not possible to accurately predict which patients will not benefit from the surgery, and thus numerous subjects undergo surgeries which will not improve their outcome, exposign them to additional health risks.

### SUMMARY

As described herein, the inventors have identified gene signatures which permit the identification of patients who will benefit from (e.g. have optimal outcomes) cytoreductive surgery as treatment for ovarian cancer. Accordingly, provided herein are methods of treatment, assays, and systems relating to ovarian cancer and the administration of cytoreductive surgery.

In one aspect, the technology described herein relates to a method of treatment comprising, detecting, in a sample obtained from a subject in need of treatment for ovarian cancer, the level of activation of at least one pathway selected from the group consisting of TGF-β/Smad signaling; RTK/Ras/MAPK/Egr-1 signaling; AMPK/Egr-1 signaling; and Hedgehog/Gli signaling; administering cytoreductive surgery to the subject if the level of activation is not increased relative to a reference level; and not administering cytoreductive surgery to the subject if the level of activation is increased relative to a reference level. In one aspect, the technology described herein relates to a method of treatment comprising, detecting, in a sample obtained from a subject in need of treatment for ovarian cancer, the level of expression products of at least one marker gene selected from Table 1 or Table 2; or the level of phosphorylated SMAD2 or SMAD3; administering cytoreductive surgery to the subject if the level of expression products selected from Table 1 or the level of phosphorylated SMAD2 or SMAD3 are not increased relative to a reference level or the level of expression products selected from Table 2 are not decreased relative to a reference level; and not administering cytoreductive surgery to the subject if the level of expression products selected from Table 1 or the level of phosphorylated SMAD2 or SMAD3 are increased relative to a reference level or the level of expression products selected from Table 2 are decreased relative to a reference level.

In one aspect, the technology described herein relates to an assay comprising, detecting, in a sample obtained from a subject in need of treatment for ovarian cancer, the level of activation of at least one pathway selected from the group consisting of TGF-β/Smad signaling; RTK/Ras/MAPK/Egr-1 signaling; AMPK/Egr-1 signaling; and Hedgehog/Gli signaling; wherein the subject is likely to benefit from cytoreductive surgery if the level of activation is not increased relative to a reference level; and wherein the subject is not likely to benefit from cytoreductive surgery if the level of activation is increased relative to a reference level. In one aspect, the technology described herein relates to an assay comprising, detecting, in a sample obtained from a subject in need of treatment for ovarian cancer, the level of expression products of at least one marker gene selected from Table 1 or Table 2; or the level of phosphorylated SMAD2 or SMAD3; wherein the subject is likely to benefit from cytoreductive surgery if the level of expression products selected from Table 1 or the level of phosphorylated SMAD2 or SMAD3 are not increased relative to a reference level or the level of expression products selected from Table 2 are not decreased relative to a reference level; and wherein the subject is not likely to benefit from cytoreductive surgery if the level of expression products selected from Table 1 or the level of phosphorylated SMAD2 or SMAD3 are increased relative to a reference level or the level of expression products selected from Table 2 are decreased relative to a reference level.

In one aspect, the technology described herein relates to a method of determining if a subject is likely to benefit from cytoreductive surgery, the method comprising, detecting, in a sample obtained from a subject in need of treatment for ovarian cancer, the level of activation of at least one pathway selected from the group consisting of: TGF-β/Smad signaling; RTK/Ras/MAPK/Egr-1 signaling; AMPK/Egr-1 signaling; and Hedgehog/Gli signaling; wherein the subject is likely to benefit from cytoreductive surgery if the level of activation is not increased relative to a reference level; and wherein the subject is not likely to benefit from cytoreductive surgery if the level of activation is increased relative to a reference level. In one aspect, the technology described herein relates to a method of determining if a subject is likely to benefit from cytoreductive surgery, the method comprising, detecting, in a sample obtained from a subject in need of treatment for ovarian cancer, the level of expression products of at least one marker gene selected from Table 1 or Table 2; or the level of phosphorylated SMAD2 or SMAD3; wherein the subject is likely to benefit from cytoreductive surgery if the level of expression products selected from Table 1 or the level of phosphorylated SMAD2 or SMAD3 are not increased relative to a reference level or the level of expression products selected from Table 2 are not decreased relative to a reference level; and wherein the subject is not likely to benefit from cytoreductive surgery if the level of expression products selected from Table 1 or the level of phosphorylated SMAD2 or SMAD3 are increased relative to a reference level or the level of expression products selected from Table 2 are decreased relative to a reference level.

In one aspect, the technology described herein relates to a computer system for determining if subject in need of treatment for ovarian cancer is likely to benefit from cytoreductive surgery, the system comprising: a measuring module configured to measure the level of activation of at least one pathway selected from the group consisting of: TGF-β/Smad signaling; RTK/Ras/MAPK/Egr-1 signaling; AMPK/Egr-1 signaling; and Hedgehog/Gli signaling; in a test sample obtained from a subject; a storage module configured to store output data from the measuring module; a comparison module adapted to compare the data stored on the storage module with a reference level, and to provide a retrieved content, and a display module for displaying whether the level in a test sample obtained from a subject is greater, by a statistically significant amount, than the reference level. In one aspect, the technology described herein relates to a computer system for determining if subject in need of treatment for ovarian cancer is likely to benefit from cytoreductive surgery, the system comprising: a measuring module configured to measure the level of expression products of at least one marker gene selected from Table 1 or Table 2; or the level of phosphorylated SMAD2 or SMAD3; in a test sample obtained from a subject; a storage module configured to store output data from the measuring module; a comparison module adapted to compare the data stored on the storage module with a reference level, and to provide a retrieved content, and a display module for displaying whether the level in a test sample obtained from a subject differs, by a statistically significant amount, from the reference level; wherein the subject is likely to benefit from cytoreductive surgery if the level of expression products selected from Table 1 or the level of phosphorylated SMAD2 or SMAD3 are not increased relative to a reference level or the level of expression products selected from Table 2 are not decreased relative to a reference level; and wherein the subject is not likely to benefit from cytoreductive surgery if the level of expression products selected from Table 1 or the level of phosphorylated SMAD2 or SMAD3 are increased relative to a reference level or the level of expression products selected from Table 2 are decreased relative to a reference level. In some embodiments, if the computing module determines that the level of of the marker in the test sample obtained from a subject is differs by a statistically significant amount from the reference level, the display module displays a signal indicating that the level in the sample obtained from a subject differs from the reference level. In some embodiments, the the signal indicates whether the the subject is likely to benefit from cytoreductive surgery. In some embodiments, the system further comprises creating a report based on the level of the marker.

In some embodiments of any of the foregoing aspects, the one or more marker genes is selected from the group consisting of: MMP2, TIMP3, ADAMTS1, VCL, TGFB1, SPARC, CYR61; EGR1, SMADs; GLIs, VCAN, CNY61, LOX, TAFs, ACTA2, POSTN, CXCL14, CCL13, FAP, NUAK1, PTCH1, TGFBR2; and TNFAIP6. In some embodiments of any of the foregoing aspects, the one or more marker genes is selected from the group consisting of: POSTN, CXCL14, CCL13, FAP, NUAK1, PTCH1, TGFBR2; and TNFAIP6. In some embodiments of any of the foregoing aspects, the level of the expression products of POSTN, CXCL14, CCL13, FAP, NUAK1, PTCH1, TGFBR2; and TNFAIP6 is determined. In some embodiments of any of the foregoing aspects, the expression products are mRNA expression products. In some embodiments of any of the foregoing aspects, the expression products are polypeptide expression products. In some embodiments of any of the foregoing aspects, the subject has advanced stage ovarian cancer. In some embodiments of any of the foregoing aspects, the sample is a tumor cell sample. In some embodiments of any of the foregoing aspects, the subject is a human. In some embodiments of any of the foregoing aspects, the level of expression of a marker gene product is determined using an method selected from the group consisting of RT-PCR; quantitative RT-PCR; Northern blot; microarray based expression analysis; Western blot; immunoprecipitation; enzyme-linked immunosorbent assay (ELISA); radioimmunological assay (RIA); sandwich assay; fluorescence in situ hybridization (FISH); immunohistological staining; radioimmunometric assay; immunofluoresence assay; mass spectroscopy and immunoelectrophoresis assay.

### DESCRIPTION OF THE DRAWINGS

FIGS. 1A-1B demonstrate Leave-one-dataset-out validation of performance of the new gene signature inpredicting overall survival in late stage ovarian cancer. FIG. 1A depicts a schematic overview of the described approach for prognostic signature creation. A database of the ovarian transcriptome [17] was used for developing a novel overall survival gene signature in a meta-analysis framework. In total, 6 datasets passed the training criteria (light grey boxes, see Methods for criteria) and 7 datasets were used for validation only (dark grey boxes). To first validate the methodology on the 6 large datasets used for training, a leave-one-dataset out approach was applied. Specifically, 6 different models were used and one dataset was always excluded from training. The excluded datasets, labeled 1 to 6, were then used for validation. FIG. 1B depicts graphs of risk stratification. For each dataset, a Kaplan-Meier analysis was performed in which the patients were stratified in two risk groups by a prediction model trained with the remaining 5 datasets only. For patients classified as high-risk by the prediction model, the survival is shown in solid black lines, for low-risk patients in dashed grey lines. P-values were calculated with the log-rank test and cutoffs for patient stratification were the medians of predicted risk scores in the training cohorts.

FIG. 2 demonstrates the validation of the survival signature in independent data. Risk stratification in 5 validation microarray datasets of late-stage, high-grade, serous ovarian cancer by a model trained on all 6 training datasets (FIG. 1B). The Gillet et al. validation dataset [24] is an RT-PCR dataset of genes associated with multidrug resistance. This data assayed only 12 of the 200 genes in the signature, which included however well characterized cancer genes such as APC, RBI and MMP9. The remaining 4 datasets had less than the 75 samples we required for training [11, 25, 26] or were published after the present model was finalized [14]. Solid black curves and dashed grey curves show the survival of high- and low-risk patients, respectively. The panel labeled “Early Stage TGCA” shows the performance in all 30 early stage, high grade, serous samples from the TCGA data. The panel labeled Partheen, represents testing of the model in a dataset in which survival information was only available as binary outcome (long vs. short survivors)[18]. The prediction model here estimated the probability of short time survival and its accuracy is shown with a ROC curve. This curve shows the true and the false positive rates for all possible cutoffs of the continuous prediction score. True positives are correctly classified short-term survivors.

FIGS. 3A-3B demonstrate the comparison of the present meta-analysis gene signature with existing prognostic factors and signatures proposed by the TCGA. In FIG. 3A, panels depict the analysis in which all datasets were combinated with overall survival information except TCGA for a Kaplan-Meier analysis. The TCGA dataset was excluded to get comparable prediction accuracy estimates to the TCGA and Verhaak et al. signatures [7, 16], which were trained on the TCGA data. Kaplan-Meier curves in grey x's represent patients classified as high-risk by our meta-analysis gene signature (FIGS. 1A-1B and 2); cases classified as high-risk in combination with tumor stage (III vs. IV) and debulking status (optimal vs. suboptimal) (top right panel); cases with tumor stage and debulking status only (middle left panel); cases classified as high-risk by the TCGA gene signature [7] (middle right panel); cases classified as high-risk by the Verhaak et al. survival signature (bottom left panel) [16]. The bottom right panel depicts the multivariate model proposed by Verhaak et al. using their survival signature, continuous TCGA subtype scores, as well as debulking status and tumor stage. BRCA1/BRCA2 mutation status could not be used because it was not available for any of the datasets. FIG. 3B depicts graphs comparing the Hazard Ratios of the meta-analysis gene signature with the ones of the TCGA and Verhaak et al. gene signatures in all cohorts in a forest plot.

FIG. 4 demonstrates pathway analysis of the debulking signature. A gene is labeled in dark grey when it is over-expressed in suboptimal tumors. Conversely, genes over-expressed in optimal debulked tissue are labeled in darker grey (i.e. CCFL13; PTCH1; GATA3; CYP17A1). Genes with predictive power towards poor prognosis based on the meta-analysis are highlighted with grey halo borders.

FIGS. 5A-5B demonstrate validation of selected genes associated with debulking status by qRT-PCR in the Bonomevalidation data (n=78). FIG. 5A depicts a graph of the observed fold-changes in suboptimal vs. optimal tumors and their standard errors of the 7 genes with significantly (P<0.05, Student t-test) different expression between the two groups. FIG. 5B depicts graphs of the prediction accuracy of a multivariate model in which the 8 qRT-PCR validated genes were equally weighted. The samples were stratified into groups of high and low risk for suboptimal surgery based on the tertiles of the multivariate risk score: the 33% of patients with highest risk score were classified as high-risk, the 33% with lowest risk score as low-risk and all others as medium-risk. Between the high- and low-risk groups, 78.8% of samples were classified correctly. The accuracy of the multivariate risk prediction is further shown with a ROC curve. The series in the graphs of FIG. 5A, are in order, “optimal” and “suboptimal”.

FIGS. 6A-6D demonstrate the validation of POSTN, pSmad2/3 and CXCL14 in an independent cohort by Immunohistochemistry (IHC). FIG. 6A depicts a histogram visualizing the frequency of optimal and suboptimal tumors stratified by POSTN IHC grade in an independent cohort of 177 samples. The true and false positive rates of POSTN IHC intensity scores [23] utilized for classification are further shown with a ROC curve. FIGS. 6B-6C depict the corresponding Figs. for pSmad2/3 and CXCL14. FIG. 6D depicts the prediction accuracy of the multivariate model in which the 3 IHC validated genes were equally weighted (as in FIG. 5B).

FIG. 7 depicts graphs of cutoff influence. A fixed gene signature size of 200 genes was used for all signatures described herein. These graphs demonstrate the influence of this cutoff on the prediction concordance of the overall survival signature. Each point represents the prediction concordance of a model with x genes in the corresponding dataset that was trained using the remaining datasets only.

FIG. 8 demonstrates the application of the TCGA model to the test sets shown in FIG. 2c of the TCGA paper [3]. The identical results show that the present implementation of the TCGA model is correct. Darker survival curves correspond to high risk patients, lighter curves to low risk patients.

FIGS. 9A-9C demonstrate the association of subtype and overall survival. FIG. 9A depicts a graph of all training and validation datasets excluding TCGA. FIG. 9B depicts the stratification of TCGA samples by subtype. FIG. 9C depicts Kaplan-Meier curves of the subtypes proposed by the Australian Ovarian Cancer Study Group (AOCS) in Tothill et al [6]. This analysis corresponds to FIG. 5B of the Tothill study, with the difference that here only the late-stage, high-grade, serous tumors used in the present meta-analysis are depicted.

FIG. 10 depicts pairwise correlation of the gene signature risk scores for the Meta-Analysis, TCGA and Verhaak et al. signatures. Numbers in the upper-right, triangular half of the matrix are the Pearson correlation coefficients, which were all statistically significant (P<0.05). Pairwise scatterplots of expression values are shown in the lower-left half and the expression histograms are shown on the matrix diagonal.

FIG. 11 depicts the prediction accuracy as a function of training sample sizes. The plot depicts the improvement of predictions when training sample sizes were increased. For each of the 9 shown datasets, 255 different models were trained using the remaining 8 datasets only. These 255 (2⁸−1) models correspond to all possible combinations of the 8 remaining databases. Each point in the plot represents a training dataset combination and the combination's total sample size is shown on the x-axis, its Hazard Ration (HR) in the validation data on the y-axis. This analysis showed that increasing the sample size via meta-analysis typically increased the model HRs. TCGA data was further identified as an extremely difficult validation dataset.

FIG. 12 demonstrates prediction of suboptimally debulked tumors in a leave-one-dataset-out cross-validation. The prediction model calculates for each sample a score. The higher the score, the higher the probability the tumor will be not optimally debulkable. For each dataset, the model is trained using only the remaining datasets. ROC curves visualize the true and false positive rates as a function of the probability cutoffs. AUCs significantly (P<0.05) larger than 0.5 are marked with an asterisk.

FIG. 13 depicts prediction of debulking status with the Berchuck et al. signature [1] as in FIG. 12. The Dressman data was excluded because a subset of Dressman samples was used for training.

FIG. 14 depicts prediction of debulking status with the POSTN expression alone as in FIG. 12.

FIG. 15 depicts the influence of the 200 gene cutoff on the prediction accuracy of the debulking signature. Each point represents the AUC of a model with x genes in the corresponding dataset that was trained using the remaining datasets only. For the Bonome et al. dataset, this shows the leave-one-dataset-out cross-validated performance in the 93 samples used for training.

FIG. 16 demonstrates the validation of selected genes by qRT-PCR in the Bonome validation dataset, a subset of 78 samples (39 optimal and 39 suboptimal tumors). Points represent patients, the x-axis shows the Affymetrix fRMA normalized intensities, the y-axis the qRT-PCR level. The Spearman correlation of platforms was highly significant for all genes (P<0.001).

FIG. 17 is a diagram of an exemplary embodiment of a system for performing an assay for determining the level of expression products of marker genes selected from Tables 1 and/2 and/or the level of phosphorylated SMAD2/3 in sample obtained from a subject.

FIG. 18 is a diagram of an embodiment of a comparison module as described herein.

FIG. 19 is a diagram of an exemplary embodiment of an operating system and instructions for a computing system as described herein.

### DETAILED DESCRIPTION

As described herein, the inventors have discovered that a number of genes are differentially regulated in tumors of patients with ovarian cancer who will benefit from cytoreductive surgery as compared to those subject with ovarian cancer who will not benefit from cytoreductive surgery. Accordingly, there are provided herein methods, assays, and systems relating to the prognosis, risk assessment, and treatment of subjects having ovarian cancer, particularly as relates to subjects receiving cytoreductive surgery as part of their treatment regimen for ovarian cancer.

“Ovarian cancer” refers to cancers arising in, or involving, the ovaries, e.g. in the epithelium of the ovaries. As used herein, the term “cancer” or “tumor” refers to an uncontrolled growth of cells which interferes with the normal functioning of the bodily organs and systems. A subject that has a cancer or a tumor is a subject having objectively measurable cancer cells present in the subject's body. Included in this definition are benign and malignant cancers, as well as dormant tumors or micrometastases. Cancers which migrate from their original location and seed vital organs can eventually lead to the death of the subject through the functional deterioration of the affected organs.

In some embodiments, the methods described herein relate to treating a subject having or diagnosed as having ovarian cancer. Subjects having ovarian cancer can be identified by a physician using current methods of diagnosing ovarian cancer. Symptoms and/or complications of ovarian cancer which characterize these conditions and aid in diagnosis are well known in the art and include but are not limited to, bloating, pelvic pain, difficulty eating, abdominal pain, back pain, constipation, tiredness, vaignal bleeding, weight loss, and frequent urination. Tests that may aid in a diagnosis of, e.g. ovarian cancer include, but are not limited to, transvaginal ultrasounds and serum AFP, LDH, OVA1, or CA-125 tests. A family history of cancer or exposure to risk factors for ovarian cancer can also aid in determining if a subject is likely to have ovarian cancer or in making a diagnosis of ovarian cancer.

Ovarian cancer is typically treated by cytoreductive surgery (also referred to herein as “debulking”) followed by administration of chemotherapy. As used here, “cytoreductive surgery” refers to surgical removal of at least part of the ovarian cancer tissue from a subject. Cytoreductive surgery can remove varying amounts of tumor tissue from a subject, depending upon the location and character of the tumor tissue, the health of the subject, and complicating factors which one of skill in the art can assess. In some embodiments, cytoreductive surgery can remove at least 10% of the tumor tissue, e.g. 10% or more, 20% or more, 30% or more, 40% or more, 50% or more, 60% or more, 70% or more, 80% or more, 90% or more, or 95% or more of the tumor tissue present in the subject. Some subjects will benefit from cytoreductive surgery, e.g. the combination of cytoreductive surgery and subsequent chemotherapy leads to improved outcomes (survival rates) as compared to chemotherapy alone. However, some subjects do not benefit from cytoreductive surgery, e.g. the combination of cytoreductive surgery and subsequent chemotherapy does not lead to improved outcomes (survival rates) as compared to chemotherapy alone. In some embodiments, “benefiting from cytroreductive surgery” can refer to a subject who will have an optimal outcome from cytoreductive surgery, e.g. no residual macroscopic tumor remains after surgery and/or the residual tumor mass after surgery is not greater than 1 cm.

As described herein, the inventors have identified certain genes which are upregulated to a statistically significant degree, as compared to a reference level, in ovarian cancer tissue of subjects who will not benefit from cytoreductive surgery. These genes are sometimes referred to herein as marker genes to indicate their relation to being a marker for whether cytoreductive surgery will be efficacious. Accordingly, some embodiments of the invention are generally related to assays, methods and systems for assessing the likely response of a subject to cytoreductive surgery and/or treating a subject for ovarian cancer, e.g. by determining if a subject will benefit from cytoreductive surgery and performing the surgery if it is likely to be efficacious. In certain embodiments, the assays, methods and systems are directed to determination of the expression level of a gene product (e.g. protein and/or gene transcript such as mRNA) in a biological sample of a subject. In certain embodiments the assays, methods, and systems are directed to determination of the expression level of a gene product of at least two genes in a biological sample of a subject, i.e. at least two genes, at least three genes, at least four genes, at least five genes, at least six genes, at least seven genes, at least eight genes, at least nine genes, at least 10 genes . . . at least 15 genes, . . . at least 25 genes, . . . at least 30 genes, or more genes, or any number of genes selected from Table 1 and/or Table 2 as described herein. In some embodiments, the marker gene(s) is selected from the group consisting of MMP2, TIMP3, ADAMTS1, VCL, TGFB1, SPARC, CYR61; EGR1, SMADs; GLIB, VCAN, CNY61, LOX, TAFs, ACTA2, POSTN, CXCL14, CCL13, FAP, NUAK1, PTCH1, TGFBR2; and TNFAIP6. In some embodiments, the marker gene(s) is selected from the group consisting of POSTN, CXCL14, CCL13, FAP, NUAK1, PTCH1, TGFBR2; and TNFAIP6. In some embodiments, the marker is the level of phosphorylated SMAD2 and/or SMAD3. In some embodiments, the assays, methods, and systems described herein are directed to determination of the expression level of a gene product of at least two genes in a biological sample of a subject, e.g. at least two genes, or at least three genes, or at least four genes, or, e.g. all of the following genes: POSTN, CXCL14, CCL13, FAP, NUAK1, PTCH1, TGFBR2; and TNFAIP6.

TABLE 1
Genes upregulated in tumors from patients who will
not benefit from cytoreductive surgery

| | Mean fold |
|---|---|
| | change |
| | (suboptimal |
| Genes | versus optimal) |
| POSTN | 1.833732822 |
| LUM | 1.504577198 |
| CXCL14 | 1.4794835 |
| NNMT | 1.449301812 |
| ASPN | 1.437637881 |
| FAP | 1.422880475 |
| LOX | 1.398395566 |
| VCAN | 1.353922244 |
| COL5A1 | 1.334666862 |
| COL3A1 | 1.311635317 |
| NUAK1 | 1.294585635 |
| CRISPLD2 | 1.285580861 |
| ACTA2 | 1.272345163 |
| CYR61 | 1.256290876 |
| MMP2 | 1.249974628 |
| ALDH1A3 | 1.244818923 |
| MICAL2 | 1.238358878 |
| CTSK | 1.232425006 |
| TGFBI | 1.222209599 |
| FOS | 1.218229847 |
| SRPX | 1.214818064 |
| OLFML2B | 1.209725412 |
| ADAMTS1 | 1.202775994 |
| TNFAIP6 | 1.199564217 |
| KIAA1199 | 1.198031989 |
| ST6GALNAC5 | 1.187271908 |
| SCRG1 | 1.181255614 |
| VSIG4 | 1.178825502 |
| PRSS23 | 1.178171225 |
| MTDH | 1.177018732 |
| C9orf3 | 1.174438495 |
| KLF6 | 1.173996511 |
| RGCC | 1.16791048 |
| RGS4 | 1.167738964 |
| ITGBS | 1.167384936 |
| TGFBR2 | 1.167308735 |
| PRRX1 | 1.167233635 |
| CH25H | 1.16682987 |
| ENPP1 | 1.166201043 |
| C5AR1 | 1.162876728 |
| LMCD1 | 1.162357335 |
| EGR1 | 1.158603956 |
| EPAS1 | 1.157219985 |
| FERMT2 | 1.154160239 |
| ABCA1 | 1.153394012 |
| SPARC | 1.153207964 |
| EMP1 | 1.148516622 |
| MTSS1 | 1.148463622 |
| MYOF | 1.147126571 |
| LGALS1 | 1.146794661 |
| PVRL2 | 1.138923059 |
| C3AR1 | 1.131180536 |
| CNN3 | 1.128940407 |
| SCG2 | 1.128561535 |
| SH3PXD2A | 1.12621939 |
| SVIL | 1.124308105 |
| DDR2 | 1.124004491 |
| SORT1 | 1.123592657 |
| ARL4C | 1.122734396 |
| LIMA1 | 1.114645998 |
| AP2S1 | 1.111424328 |
| PTS | 1.110404975 |
| VCL | 1.110357376 |
| GSN | 1.108808893 |
| SUMO3 | 1.108363487 |
| LPAR1 | 1.1072977 |
| STK3 | 1.102196233 |
| CD82 | 1.102165973 |
| SRPK2 | 1.099476961 |
| TMC6 | 1.098976244 |
| SMAD1 | 1.097703625 |
| ZNF706 | 1.092371262 |
| EHD4 | 1.092239946 |
| KIAA0196 | 1.090551191 |
| VTI1B | 1.089441079 |
| TRIM8 | 1.088712121 |
| TMEM165 | 1.0881192 |
| WSB2 | 1.085779937 |
| LGMN | 1.084746285 |
| ACTR1A | 1.084541033 |
| LHFPL2 | 1.074203835 |
| HSPA14 | 1.07386303 |
| MPP5 | 1.071834461 |
| AZIN1 | 1.07176981 |
| ATP2B1 | 1.071597159 |
| ATP6V1C1 | 1.071278388 |
| MAN2A1 | 1.07119714 |
| TM9SF3 | 1.071103021 |
| CREM | 1.069005736 |
| RAB32 | 1.062484406 |
| ACSL3 | 1.061239671 |
| MARK3 | 1.057014582 |
| SNX19 | 1.056175078 |
| PCMT1 | 1.055963962 |
| PDCD10 | 1.053423869 |
| SLC35C1 | 1.052840209 |
| GRB2 | 1.048009157 |
| MANBA | 1.047157445 |
| SOS2 | 1.044111399 |
| SYPL1 | 1.035303895 |
| DNAJB12 | 1.032048575 |
| PPM1A | 1.020652923 |

TABLE 2
Genes downregulated in tumors in patients who will
not benefit from cytoreductive surgery

| | Mean fold |
|---|---|
| | change |
| | (suboptimal |
| Genes | versus optimal) |
| DLK1 | −1.534738165 |
| PEG3 | −1.435544005 |
| COLEC11 | −1.412092206 |
| STAR | −1.33055129 |
| PROM1 | −1.287199118 |
| CCL13 | −1.273289194 |
| PCSK6 | −1.220397348 |
| NRTN | −1.218426513 |
| GREB1 | −1.217899277 |
| AMT | −1.164533301 |
| SEZ6L2 | −1.161941676 |
| CDHR1 | −1.152500471 |
| TRIM58 | −1.143368805 |
| COL7A1 | −1.142656189 |
| CHGA | −1.142064127 |
| IL17RB | −1.141071725 |
| HMGCS2 | −1.13824265 |
| CBR4 | −1.136770677 |
| TNFRSF14 | −1.132818482 |
| CHST2 | −1.131675239 |
| NEK11 | −1.130825409 |
| PAQR6 | −1.128503382 |
| CDK10 | −1.125593318 |
| GATA4 | −1.122456596 |
| WDR59 | −1.12206389 |
| C4BPA | −1.115789378 |
| AP3B2 | −1.108271779 |
| BAIAP3 | −1.107107502 |
| MACROD1 | −1.106647952 |
| PTCH1 | −1.10573423 |
| PIGL | −1.104995288 |
| CYP17A1 | −1.104705569 |
| GAS8 | −1.103814526 |
| RIC3 | −1.102545943 |
| IGF1R | −1.101267576 |
| CYP27B1 | −1.099907089 |
| CHST10 | −1.098393606 |
| SOX15 | −1.09766276 |
| NAV2 | −1.097588672 |
| APRT | −1.09691552 |
| PDPR | −1.095990518 |
| ZNHIT3 | −1.095572941 |
| NOL3 | −1.092920888 |
| CYP2B6 | −1.092605991 |
| GRB14 | −1.090923585 |
| PPOX | −1.090340515 |
| ZNF652 | −1.090330929 |
| TTLL1 | −1.089906054 |
| KIAA0240 | −1.088979818 |
| RANBP10 | −1.088228957 |
| TTLL12 | −1.087588361 |
| PARD6A | −1.086371608 |
| SOX3 | −1.085666199 |
| SPG7 | −1.085208603 |
| CUL7 | −1.084841041 |
| FSD1 | −1.08304669 |
| FGFR4 | −1.080681201 |
| CACNA1F | −1.079909535 |
| NFATC3 | −1.076213226 |
| KIAA0753 | −1.074786644 |
| TMEM74B | −1.069597468 |
| NEIL1 | −1.069013172 |
| RBM8A | −1.068930671 |
| ULK2 | −1.068293104 |
| FAN1 | −1.067889474 |
| NFYA | −1.066889968 |
| ACD | −1.066706864 |
| CUL9 | −1.066208523 |
| CHMP1A | −1.066129874 |
| FMO5 | −1.065647977 |
| REV1 | −1.065332757 |
| LLGL1 | −1.064270705 |
| UCN | −1.063565091 |
| PIDD | −1.061836093 |
| WDR33 | −1.060345186 |
| MAZ | −1.060154848 |
| SCIN | −1.058206404 |
| SLC4A3 | −1.058190114 |
| NAT2 | −1.054164325 |
| EPB41 | −1.054017306 |
| SEMA4G | −1.052201524 |
| TBXA2R | −1.051393897 |
| SMPD3 | −1.05072838 |
| RNMT | −1.049949549 |
| PPP2R5D | −1.04910143 |
| NR1H4 | −1.047110522 |
| OR1D2 | −1.045170076 |
| B4GALNT1 | −1.044675551 |
| ARFIP2 | −1.043495791 |
| FHOD1 | −1.041985958 |
| HSF4 | −1.041698247 |
| HAP1 | −1.039060694 |
| TMEM53 | −1.039059472 |
| PLG | −1.037745292 |
| SPATA20 | −1.03753861 |
| SCARB1 | −1.033284155 |
| THOC1 | −1.033031491 |
| FABP7 | −1.032055261 |

The gene names listed in Tables 1 and 2 are common names. NCBI Gene ID numbers for each of the genes listed in Tables 1 and 2 can be obtained by searching the “Gene” Database of the NCBI (available on the World Wide Web at http://www.ncbi.nlm.nih.gov/) using the common name as the query and selecting the first returned Homo sapiens gene.

In some embodiments, the methods and assays described herein include (a) transforming the gene expression product into a detectable gene target; (b) measuring the amount of the detectable gene target; and (c) comparing the amount of the detectable gene target to an amount of a reference, wherein if the amount of the detectable gene target is statistically significantly greater than the amount of the reference level, the subject is identified as not likely to benefit from and/or is not administered cytoreductive surgery. In some embodiments, if the amount of the detectable gene target is not statistically significantly greater than the amount of the reference level, the subject is identified as likely to benefit from and/or is administered cytoreductive surgery.

In some embodiments, the reference can be a level of expression of the marker gene product in a population of subjects who have been demonstrated to benefit from cytoreductive surgery. In some embodiments, the reference can also be a level of expression of the marker gene product in a control sample, a pooled sample of control individuals or a numeric value or range of values based on the same.

In certain embodiments, the marker gene(s) are selected from the genes listed in Tables 1 and/or 2. In certain embodiments, one or more marker genes are selected from the group consisting of POSTN, CXCL14, CCL13, FAP, NUAK1, PTCH1, TGFBR2; and TNFAIP6. In some embodiments, if the marker gene(s) are not upregulated as compared to the reference, the subject is determined to be likely to benefit from cytoreductive surgery and/or is administered cytoreductive surgery. In some embodiments, if the marker gene(s) are upregulated as compared to the reference, the subject is determined to not be likely to benefit from cytoreductive surgery and/or is not administered cytoreductive surgery. Preferably, one looks at the presence and/or absence of a statistically significant change. For example, even if a few genes in a group do not differ from normal, a subject can be identified as not being likely to benefit from cytoreductive surgery if the overall change of the group shows a significant change, preferably a statistically significant change.

In certain embodiments, the marker gene(s) are selected from the genes listed in Table 1 and/or Table 2. In certain embodiments, one or more marker genes are selected from the group consisting of POSTN, CXCL14, CCL13, FAP, NUAK1, PTCH1, TGFBR2; and TNFAIP6. In subjects who are not likely to benefit from cytoreductive surgery, the marker genes listed in Table 1 can be upregulated and those in Table 2 can be downregulated, e.g. for marker genes listed in Table 1, if the measured marker gene expression in a subject is higher as compared to a reference level of that marker gene's expression, then the subject is identified as not likely to benefit from cytoreductive surgery. Likewise, for marker genes listed in Table 2, if the measured marker gene expression in a subject is lower as compared to a reference level of that marker gene's expression, then the subject is identified not likely to benefit from cytoreductive surgery. Preferably, once looks at a statistically significant change. However, even if a few genes in a group do not differ from normal, a subject can be identified as likely to not benefit from cytoreductive surgery if the overall change of the group shows a significant change, preferably a statistically significant change.

The level of a gene expression product of a marker gene in Table 1 which is higher than a reference level of that marker gene by at least about 10% than the reference amount, at least about 20%, at least about 30%, at least about 40%, at least about 50%, at least about 80%, at least about 100%, at least about 200%, at least about 300%, at least about 500% or at least about 1000% or more, is indicative that the subject is not likely to benefit from cytoreductive surgery and/or is not administered cytoreductive surgery in accordance with the methods described herein.

The level of a gene expression product of a marker gene in Table 2 which is lower than a reference level of that marker gene by at least about 10% than the reference amount, at least about 20%, at least about 30%, at least about 40%, at least about 50%, at least about 80%, at least about 90% or more, is indicative that the subject is not likely to benefit from cytoreductive surgery and/or is not administered cytoreductive surgery in accordance with the methods described herein.

By way of non-limiting example, Table 7 depicts non-limiting potential combinations of two marker genes that can be used in the methods and assays described herein. All possible combinations of 2 or more of the indicated markers are contemplated herein.

TABLE 7

| | POSTN | CXCL14 | CCL13 | FAP | NUAK | PTCH1 | TGFBR2 | TNFAIP6 | pSmad2/3 |
|---|---|---|---|---|---|---|---|---|---|
| POSTN | | X | X | X | X | X | X | X | X |
| CXCL14 | X | | X | X | X | X | X | X | X |
| CCL13 | X | X | | X | X | X | X | X | X |
| FAP | X | X | X | | X | X | X | X | X |
| NUAK | X | X | X | X | | X | X | X | X |
| PTCH1 | X | X | X | X | X | | X | X | X |
| TGFBR2 | X | X | X | X | X | X | | X | X |
| TNFAIP6 | X | X | X | X | X | X | X | | X |
| pSmad2/3 | X | X | X | X | X | X | X | X | |

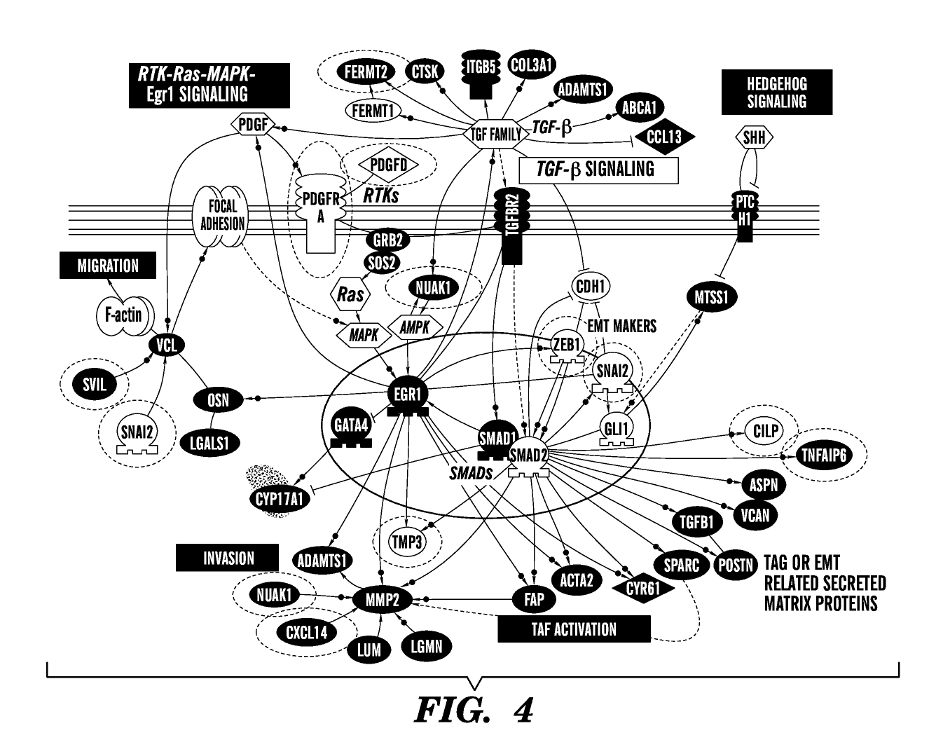

In one aspect, the technology described herein relates to a method of treatment comprising detecting, in a sample obtained from a subject in need of treatment for ovarian cancer, the level of activation of at least one pathway selected from the group consisting of TGF-β/Smad signaling; RTK/Ras/MAPK/Egr-1 signaling; AMPK/Egr-1 signaling; and Hedgehog/Gli signaling, administering cytoreductive surgery to the subject if the level of activation is not increased relative to a reference level; and not administering cytoreductive surgery to the subject if the level of activation is increased relative to a reference level. “Activation” of a given pathway can include increased levels of expression products of one or more genes comprising the pathway, preferably two or more genes (e.g. two genes, three genes, four genes, five genes, or more genes of the pathway) and/or increased levels of activity of of the proteins comprising the pathway, e.g. increased phosphorylation of a target and/or member of the pathway or increased transcription of a gene whose expression is regulated by the pathway. These pathways and their constituent genes are known in the art. FIG. 4 depicts key aspects of each of these pathways. By way of non-limiting example, TGFβ/Smad signaling can include FERMT2, CTSK, ITGBS, CCL3A1, ADAMTS1, ABCA1, FERMT1, CCL13, TGFBR2, NUAK1, AMPK, CDH1, EGR1, SMAD1, and SMAD2. By way of non-limiting example, RTK/Ras/MAPK/Egr-1 signaling can include PDGF, RTKs, PDFGRA, GRB2, SOS2, Ras, MAPK, and EGR1. By way of non-limiting example, AMPK/Egr-1 signaling can include NUAK1, AMPK, and EGR1. By way of non-limiting example, Hedgehog/Gli signaling can include PTCH1, MTSS1, GLI1, SMAD2, and SNAI2.

In some embodiments of any of the aspects described herein, the level of at least one expression products of each of at least 2 of the four pathways described in the preceding pathway are determined. In some embodiments of any of the aspects described herein, the level of at least one expression products of each of at least 3 of the four pathways described in the preceding pathway are determined. In some embodiments of any of the aspects described herein, the level of at least one expression products of each of the four pathways described in the preceding pathway are determined.

As used herein, the term “transforming” or “transformation” refers to changing an object or a substance, e.g., biological sample, nucleic acid or protein, into another substance. The transformation can be physical, biological or chemical. Exemplary physical transformation includes, but not limited to, pre-treatment of a biological sample, e.g., from whole blood to blood serum by differential centrifugation. A biological/chemical transformation can involve at least one enzyme and/or a chemical reagent in a reaction. For example, a DNA sample can be digested into fragments by one or more restriction enzyme, or an exogenous molecule can be attached to a fragmented DNA sample with a ligase. In some embodiments, a DNA sample can undergo enzymatic replication, e.g., by polymerase chain reaction (PCR).

Methods to measure gene expression products associated with the marker genes described herein are well known to a skilled artisan. Such methods to measure gene expression products, e.g., protein level, include ELISA (enzyme linked immunosorbent assay), western blot, and immunoprecipitation, immunofluorescence using detection reagents such as an antibody or protein binding agents. Alternatively, a peptide can be detected in a subject by introducing into a subject a labeled anti-peptide antibody and other types of detection agent. For example, the antibody can be labeled with a radioactive marker whose presence and location in the subject is detected by standard imaging techniques.

For example, antibodies for the polypeptide expression products of the marker genes described herein are commercially available and can be used for the purposes of the invention to measure protein expression levels, e.g. anti-CXCL14 (Cat. No. ab46010; Abcam; Cambridge, Mass.) and anti-POSTN (Cat No. LF-PA0075; BioVendor R&D; Asheville, N.C.). Alternatively, since the amino acid sequences for the marker genes described herein are known and publically available at NCBI website, one of skill in the art can raise their own antibodies against these proteins of interest for the purpose of the invention. Furthermore, antibodies specific for certain isoforms, e.g. phosphorylated SMAD2/3 are commercially available, e.g. anti-pSmad2/3 (Cat No 3101; Cell Signaling; Danvers, Mass.).

The amino acid sequences of the marker genes described herein have been assigned NCBI accession numbers for different species such as human, mouse and rat. In particular, the NCBI accession numbers for the amino acid sequences of the human marker genes are included herein. For the human POSTN protein (e.g. NCBI Ref Seq: NP_{—}006466; SEQ ID NO: 1); the human CXCL14 protein (e.g. NCBI Ref Seq: NP_{—}004878; SEQ ID NO: 2); the human CCL13 protein (e.g. NCBI Ref Seq: NP_; SEQ ID NO: 3); the human FAP protein (e.g. NCBI Ref Seq: NP_{—}004451; SEQ ID NO: 4); the human NUAK1 protein (e.g. NCBI Ref Seq: NP_{—}055655; SEQ ID NO: 5); the human PTCH1 protein (e.g. NCBI Ref Seq: NP_{—}000255; SEQ ID NO: 6); the human TGFBR2 protein (e.g. NCBI Ref Seq: NP_{—}001020018; SEQ ID NO: 7); the human TNFAIP6 protein (e.g. NCBI Ref Seq: NP 009046; SEQ ID NO: 8); the human Smad2 protein (e.g. NCBI Ref Seq: NP 001003652; SEQ ID NO: 9); and the human Smad3 protein (e.g. NCBI Ref Seq: NP_{—}005893; SEQ ID NO: 10) are known.

In some embodiments, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations Immunochemistry is a family of techniques based on the use of an antibody, wherein the antibodies are used to specifically target molecules inside or on the surface of cells. The antibody typically contains a marker that will undergo a biochemical reaction, and thereby experience a change color, upon encountering the targeted molecules. In some instances, signal amplification can be integrated into the particular protocol, wherein a secondary antibody, that includes the marker stain or marker signal, follows the application of a primary specific antibody.

In some embodiments, the assay can be a Western blot analysis. Alternatively, proteins can be separated by two-dimensional gel electrophoresis systems. Two-dimensional gel electrophoresis is well known in the art and typically involves iso-electric focusing along a first dimension followed by SDS-PAGE electrophoresis along a second dimension. These methods also require a considerable amount of cellular material. The analysis of 2D SDS-PAGE gels can be performed by determining the intensity of protein spots on the gel, or can be performed using immune detection. In other embodiments, protein samples are analyzed by mass spectroscopy.

Immunological tests can be used with the methods and assays described herein and include, for example, competitive and non-competitive assay systems using techniques such as Western blots, radioimmunoassay (RIA), ELISA (enzyme linked immunosorbent assay), “sandwich” immunoassays, immunoprecipitation assays, immunodiffusion assays, agglutination assays, e.g. latex agglutination, complement-fixation assays, immunoradiometric assays, fluorescent immunoassays, e.g. FIA (fluorescence-linked immunoassay), chemiluminescence immunoassays (CLIA), electrochemiluminescence immunoassay (ECLIA, counting immunoassay (CIA), lateral flow tests or immunoassay (LFIA), magnetic immunoassay (MIA), and protein A immunoassays. Methods for performing such assays are known in the art, provided an appropriate antibody reagent is available. In some embodiment, the immunoassay can be a quantitative or a semi-quantitative immunoassay.

An immunoassay is a biochemical test that measures the concentration of a substance in a biological sample, typically a fluid sample such as serum, using the interaction of an antibody or antibodies to its antigen. The assay takes advantage of the highly specific binding of an antibody with its antigen. For the methods and assays described herein, specific binding of the target polypeptides with respective proteins or protein fragments, or an isolated peptide, or a fusion protein described herein occurs in the immunoassay to form a target protein/peptide complex. The complex is then detected by a variety of methods known in the art. An immunoassay also often involves the use of a detection antibody.

Enzyme-linked immunosorbent assay, also called ELISA, enzyme immunoassay or EIA, is a biochemical technique used mainly in immunology to detect the presence of an antibody or an antigen in a sample. The ELISA has been used as a diagnostic tool in medicine and plant pathology, as well as a quality control check in various industries.

In one embodiment, an ELISA involving at least one antibody with specificity for the particular desired antigen (i.e. a marker gene polypeptide as described herein) can also be performed. A known amount of sample and/or antigen is immobilized on a solid support (usually a polystyrene micro titer plate). Immobilization can be either non-specific (e.g., by adsorption to the surface) or specific (e.g. where another antibody immobilized on the surface is used to capture antigen or a primary antibody). After the antigen is immobilized, the detection antibody is added, forming a complex with the antigen. The detection antibody can be covalently linked to an enzyme, or can itself be detected by a secondary antibody which is linked to an enzyme through bio-conjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

In another embodiment, a competitive ELISA is used. Purified antibodies that are directed against a target polypeptide or fragment thereof are coated on the solid phase of multi-well plate, i.e., conjugated to a solid surface. A second batch of purified antibodies that are not conjugated on any solid support is also needed. These non-conjugated purified antibodies are labeled for detection purposes, for example, labeled with horseradish peroxidase to produce a detectable signal. A sample (e.g., tumor, blood, serum or plasma) from a subject is mixed with a known amount of desired antigen (e.g., a known volume or concentration of a sample comprising a target polypeptide) together with the horseradish peroxidase labeled antibodies and the mixture is then are added to coated wells to form competitive combination. After incubation, if the polypeptide level is high in the sample, a complex of labeled antibody reagent-antigen will form. This complex is free in solution and can be washed away. Washing the wells will remove the complex. Then the wells are incubated with TMB (3,3′,5,5′-tetramethylbenzidene) color development substrate for localization of horseradish peroxidase-conjugated antibodies in the wells. There will be no color change or little color change if the target polypeptide level is high in the sample. If there is little or no target polypeptide present in the sample, a different complex in formed, the complex of solid support bound antibody reagents-target polypeptide. This complex is immobilized on the plate and is not washed away in the wash step. Subsequent incubation with TMB will produce much color change. Such a competitive ELSA test is specific, sensitive, reproducible and easy to operate.

There are other different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904. These references are hereby incorporated by reference in their entirety.

In one embodiment, the levels of a polypeptide in a sample can be detected by a lateral flow immunoassay test (LFIA), also known as the immunochromatographic assay, or strip test. LFIAs are a simple device intended to detect the presence (or absence) of antigen, e.g. a polypeptide, in a fluid sample. There are currently many LFIA tests are used for medical diagnostics either for home testing, point of care testing, or laboratory use. LFIA tests are a form of immunoassay in which the test sample flows along a solid substrate via capillary action. After the sample is applied to the test strip it encounters a colored reagent (generally comprising antibody specific for the test target antigen) bound to microparticles which mixes with the sample and transits the substrate encountering lines or zones which have been pretreated with another antibody or antigen. Depending upon the level of target polypeptides present in the sample the colored reagent can be captured and become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water, and/or homogenized tumor samples etc. Strip tests are also known as dip stick test, the name bearing from the literal action of “dipping” the test strip into a fluid sample to be tested. LFIA strip tests are easy to use, require minimum training and can easily be included as components of point-of-care test (POCT) diagnostics to be use on site in the field. LFIA tests can be operated as either competitive or sandwich assays. Sandwich LFIAs are similar to sandwich ELISA. The sample first encounters colored particles which are labeled with antibodies raised to the target antigen. The test line will also contain antibodies to the same target, although it may bind to a different epitope on the antigen. The test line will show as a colored band in positive samples. In some embodiments, the lateral flow immunoassay can be a double antibody sandwich assay, a competitive assay, a quantitative assay or variations thereof. Competitive LFIAs are similar to competitive ELISA. The sample first encounters colored particles which are labeled with the target antigen or an analogue. The test line contains antibodies to the target/its analogue. Unlabelled antigen in the sample will block the binding sites on the antibodies preventing uptake of the colored particles. The test line will show as a colored band in negative samples. There are a number of variations on lateral flow technology. It is also possible to apply multiple capture zones to create a multiplex test.

The use of “dip sticks” or LFIA test strips and other solid supports have been described in the art in the context of an immunoassay for a number of antigen biomarkers. U.S. Pat. Nos. 4,943,522; 6,485,982; 6,187,598; 5,770,460; 5,622,871; 6,565,808, U.S. patent application Ser. No. 10/278,676; U.S. Ser. No. 09/579,673 and U.S. Ser. No. 10/717,082, which are incorporated herein by reference in their entirety, are non-limiting examples of such lateral flow test devices. Examples of patents that describe the use of “dip stick” technology to detect soluble antigens via immunochemical assays include, but are not limited to U.S. Pat. Nos. 4,444,880; 4,305,924; and 4,135,884; which are incorporated by reference herein in their entireties. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. It is within the skill of one in the art to modify the teachings of this “dip stick” technology for the detection of polypeptides using antibody reagents as described herein.

Other techniques can be used to detect the level of a polypeptide in a sample. One such technique is the dot blot, and adaptation of Western blotting (Towbin et at., Proc. Nat. Acad. Sci. 76:4350 (1979)). In a Western blot, the polypeptide or fragment thereof can be dissociated with detergents and heat, and separated on an SDS-PAGE gel before being transferred to a solid support, such as a nitrocellulose or PVDF membrane. The membrane is incubated with an antibody reagent specific for the target polypeptide or a fragment thereof. The membrane is then washed to remove unbound proteins and proteins with non-specific binding. Detectably labeled enzyme-linked secondary or detection antibodies can then be used to detect and assess the amount of polypeptide in the sample tested. The intensity of the signal from the detectable label corresponds to the amount of enzyme present, and therefore the amount of polypeptide. Levels can be quantified, for example by densitometry.

In certain embodiments, the gene expression products as described herein can be instead determined by determining the level of messenger RNA (mRNA) expression of genes associated with the marker genes described herein. Such molecules can be isolated, derived, or amplified from a biological sample, such as a tumor biopsy. Detection of mRNA expression is known by persons skilled in the art, and comprise, for example but not limited to, PCR procedures, RT-PCR, Northern blot analysis, differential gene expression, RNA protection assay, microarray analysis, hybridization methods etc.

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e. each primer is specifically designed to be complementary to a strand of the genomic locus to be amplified. In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

The nucleic acid sequences of the marker genes described herein have been assigned NCBI accession numbers for different species such as human, mouse and rat. In particular, the NCBI accession numbers for the nuclei acid sequences of the human marker genes are included herein. For the human POSTN mRNA (e.g. NCBI Ref Seq: NM_{—}006475; SEQ ID NO: 11); the human CXCL14 mRNA (e.g. NCBI Ref Seq: NM_{—}004887; SEQ ID NO: 12); the human CCL13 mRNA (e.g. NCBI Ref Seq: NM_{—}005408; SEQ ID NO: 13); the human FAP mRNA (e.g. NCBI Ref Seq: NM_{—}004460; SEQ ID NO: 14); the human NUAK1 mRNA (e.g. NCBI Ref Seq: NM_{—}014840; SEQ ID NO: 15); the human PTCH1 mRNA (e.g. NCBI Ref Seq: NM_{—}000264; SEQ ID NO: 16); the human TGFBR2 mRNA (e.g. NCBI Ref Seq: NM_{—}001024847; SEQ ID NO: 17); and the human TNFAIP6 mRNA (e.g. NCBI Ref Seq: NM_{—}007115; SEQ ID NO: 18) are known.

Accordingly, a skilled artisan can design an appropriate primer based on the known sequence for determining the mRNA level of the respective gene.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In general, the PCR procedure describes a method of gene amplification which is comprised of (i) sequence-specific hybridization of primers to specific genes within a nucleic acid sample or library, (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In some embodiments, one or more of the reagents (e.g. an antibody reagent and/or nucleic acid probe) described herein can comprise a detectable label and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents (e.g. antibodies and nucleic acid probes) are well known in the art.

In some embodiments, detectable labels can include labels that can be detected by spectroscopic, photochemical, biochemical, immunochemical, electromagnetic, radiochemical, or chemical means, such as fluorescence, chemifluorescence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable e.g., as is common in immunological labeling using secondary and tertiary antibodies), The detectable label can be linked by covalent or non-covalent means to the reagent. Alternatively. a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection reagent is label with a fluorescent compound. When the fluorescently labeled antibody is exposed to light of the proper wavelength, its presence can then be detected due to fluorescence. In some embodiments, a detectable label can be a fluorescent dye molecule, or fluorophore including, but not limited to fluorescein, phycoerythrin, phycocyanin, o-phthaldehyde, fluorescamine, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfiuorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g. umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. In some embodiments, a detectable label can be a radiolabel including, but not limited to ³H, ¹²⁵I, ³⁵S, ¹⁴C, ³²P, and ³³P. In some embodiments, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments, a detectable label is a chemiluminescent label, including, but not limited to lucigenin, luminol, luciferin, isoluminol, theromatic acridinium ester, imidazole, acridinium salt and oxalate ester. In some embodiments, a detectable label can be a spectral colorimetric label including, but not limited to colloidal gold or colored glass or plastic (e.g., polystyrene, polypropylene, and latex) beads.

In some embodiments, detection reagents can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i. e. specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e. g. from DAKO; Carpinteria, Calif. A reagent can also be detectably labeled using fluorescence emitting metals such as ¹⁵²Eu, or others of the lanthanide series. These metals can be attached to the reagent using such metal chelating groups as diethylenetriaminepentaacetic acid (DTPA) or ethylenediaminetetraacetic acid (EDTA).

In some embodiments of any of the aspects described herein, the level of expression products and/or phorphorylation level of more than one gene can be determined simultaneously (e.g. a multiplex assay) or in parallel. In some embodiments, the level of expression products and/or phorphorylation level of no more than 200 other genes is determined. In some embodiments, the level of expression products and/or phorphorylation level of no more than 100 other genes is determined. In some embodiments, the level of expression products and/or phorphorylation level of no more than 20 other genes is determined. In some embodiments, the sequence, expression level, and/or mutational status of no more than 10 other genes is determined.

The term “sample” or “test sample” as used herein denotes a sample taken or isolated from a biological organism, e.g., a tumor sample from a subject. Exemplary biological samples include, but are not limited to, a biofluid sample; serum; plasma; urine; saliva; a tumor sample; a tumor biopsy and/or tissue sample etc. The term also includes a mixture of the above-mentioned samples. The term “test sample” also includes untreated or pretreated (or pre-processed) biological samples. In some embodiments, a test sample can comprise cells from subject. In some embodiments, a test sample can be a tumor cell test sample, e.g. the sample can comprise cancerous cells, cells from a tumor, and/or a tumor biopsy.

The test sample can be obtained by removing a sample of cells from a subject, but can also be accomplished by using previously isolated cells (e.g. isolated at a prior timepoint and isolated by the same or another person). In addition, the test sample can be freshly collected or a previously collected sample.

In some embodiments, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments, the test sample is a clarified test sample, for example, by centrifugation and collection of a supernatant comprising the clarified test sample. In some embodiments, a test sample can be a pre-processed test sample, for example, supernatant or filtrate resulting from a treatment selected from the group consisting of centrifugation, filtration, thawing, purification, and any combinations thereof. In some embodiments, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments, the methods, assays, and systems described herein can further comprise a step of obtaining a test sample from a subject. In some embodiments, the subject can be a human subject.

In some aspects, the invention described herein is directed to systems (and computer readable media for causing computer systems) for obtaining data from at least one sample obtained from at least one subject, the system comprising 1) a measuring module configured to measure the level of expression products of one or more marker genes selected from Table 1 and/or Table 2 and/or the level of phosphorylated SMAD2/3 in a test sample obtained from a subject, 2) a storage module configured to store output data from the measuring module, 3) a comparison module adapted to compare the data stored on the storage module with a reference level, and to provide a retrieved content, and 4) a display module for displaying whether the expression level of the one or more marker genes and/or the level of phosphorylated SMAD2/3 is greater than or less than, by a statistically significant amount, than the reference level and/or displaying the relative levels.

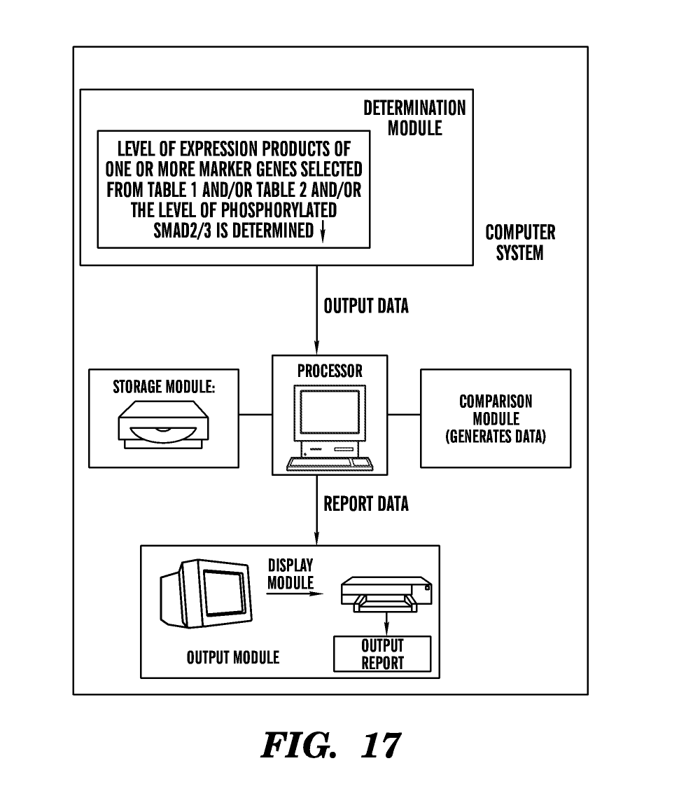

In one embodiment, provided herein is a system comprising: (a) at least one memory containing at least one computer program adapted to control the operation of the computer system to implement a method that includes 1) a measuring module configured to measure the level of expression products of one or more marker genes selected from Table 1 and/or Table 2 and/or the level of phosphorylated SMAD2/3 in a test sample obtained from a subject, 2) a storage module configured to store output data from the measuring module, 3) a computing module adapted to identify from the output data whether the level in a sample obtained from a subject is statistically significantly greater than a reference level, and 4) a display module for displaying a content based in part on the data output from the measuring module, wherein the content comprises a signal indicative of the level of expression products of one or more marker genes selected from Table 1 and/or Table 2 and/or the level of phosphorylated SMAD2/3 and (b) at least one processor for executing the computer program (see FIG. 17).

In some embodiments, the measuring module can measure the presence and/or intensity of a detectable signal from an immunoassay indicating the level of expression products of one or more marker genes selected from Table 1 and/or Table 2 and/or the level of phosphorylated SMAD2/3 in the test sample. Exemplary embodiments of a measuring module can include a FACS machine, automated immunoassay, etc. In some embodiments, the measuring module can measure the presence and/or intensity of a detectable signal from a nucleic acid probe assay indicating the level of expression products of one or more marker genes selected from Table 1 and/or Table 2.

The measuring module can comprise any system for detecting a signal elicited from an assay to determine the level of expression products of one or more marker genes selected from Table 1 and/or Table 2 and/or the level of phosphorylated SMAD2/3 as described above herein. In some embodiments, the measuring module can comprise multiple units for different functions, such as measurement of the nucleic acid expression product of a marker gene and the measurement of the level of phosphorylated SMAD2/3. In one embodiment, the measuring module can be configured to perform the methods described elsewhere herein, e.g. quantitative RT-PCR, or detection of any detectable label or signal.

The term “computer” can refer to any non-human apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

The term “computer-readable medium” may refer to any storage device used for storing data accessible by a computer, as well as any other means for providing access to data by a computer. Examples of a storage-device-type computer-readable medium include: a magnetic hard disk; a floppy disk; an optical disk, such as a CD-ROM and a DVD; a magnetic tape; a memory chip. The term a “computer system” may refer to a system having a computer, where the computer comprises a computer-readable medium embodying software to operate the computer. The term “software” is used interchangeably herein with “program” and refers to prescribed rules to operate a computer. Examples of software include: software; code segments; instructions; computer programs; and programmed logic.

The computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

Embodiments of the invention can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules can perform other functions, thus the modules are not limited to having any particular functions or set of functions.

The functional modules of certain embodiments of the invention include at minimum a measuring module, a storage module, a computing module, and a display module. The functional modules can be executed on one, or multiple, computers, or by using one, or multiple, computer networks. The measuring module has computer executable instructions to provide e.g., levels of platelet-adherent leukocytes etc. in computer readable form.

The information determined in the measuring system can be read by the storage module. As used herein the “storage module” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon, for example, sample name, biomolecule assayed and the level of said biomolecule. Such information may be provided in digital form that can be transmitted and read electronically, e.g., via the Internet, on diskette, via USB (universal serial bus) or via any other suitable mode of communication.

As used herein, “stored” refers to a process for encoding information on the storage module. Those skilled in the art can readily adopt any of the presently known methods for recording information on known media to generate manufactures comprising expression level information.

In some embodiments of any of the systems described herein, the storage module stores the output data from the measuring module. In additional embodiments, the storage module stores reference information such as levels of expression products of marker genes in in healthy subjects, and/or a population of subjects who do benefit from/receive optimal cytoreductive surgery.

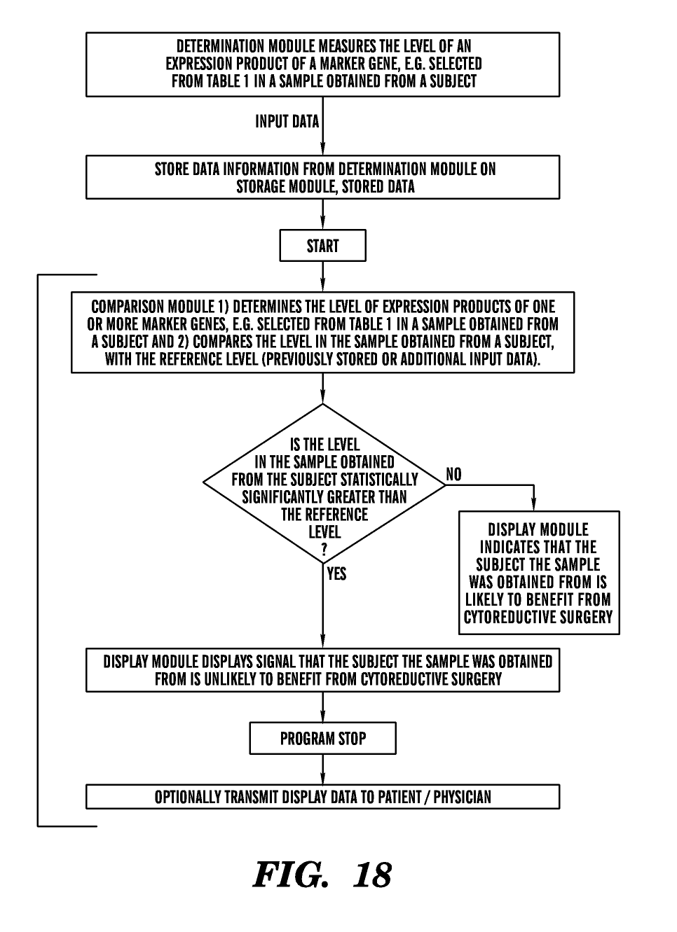

The “computing module” can use a variety of available software programs and formats for computing the level of expression products. Such algorithms are well established in the art. A skilled artisan is readily able to determine the appropriate algorithms based on the size and quality of the sample and type of data. The data analysis tools and equations described herein can be implemented in the computing module of the invention. In some embodiments, the computing module can comprise a computer and/or a computer system. In one embodiment, the computing module further comprises a comparison module, which compares the level of expression products in a sample obtained from a subject as described herein with a reference level as described herein (see, e.g. FIG. 18). By way of an example, when the level of expression products of one or more genes selected from Tables 1 and 2 in a sample obtained from a subject is measured, a comparison module can compare or match the output data with the mean level of expression products of that gene(s) in a population of subjects not having signs or symptoms of ovarian cancer and/or the level in tumor cells of subjects who received optimal results from cytoreductive surgeries (i.e. a reference level). In certain embodiments, the mean level of the expression products of one or more genes in a population of reference subjects can be pre-stored in the storage module. During the comparison or matching process, the comparison module can determine whether the level in a sample obtained from a subject is statistically significantly greater or less than the reference level. In various embodiments, the comparison module can be configured using existing commercially-available or freely-available software for comparison purpose, and may be optimized for particular data comparisons that are conducted.

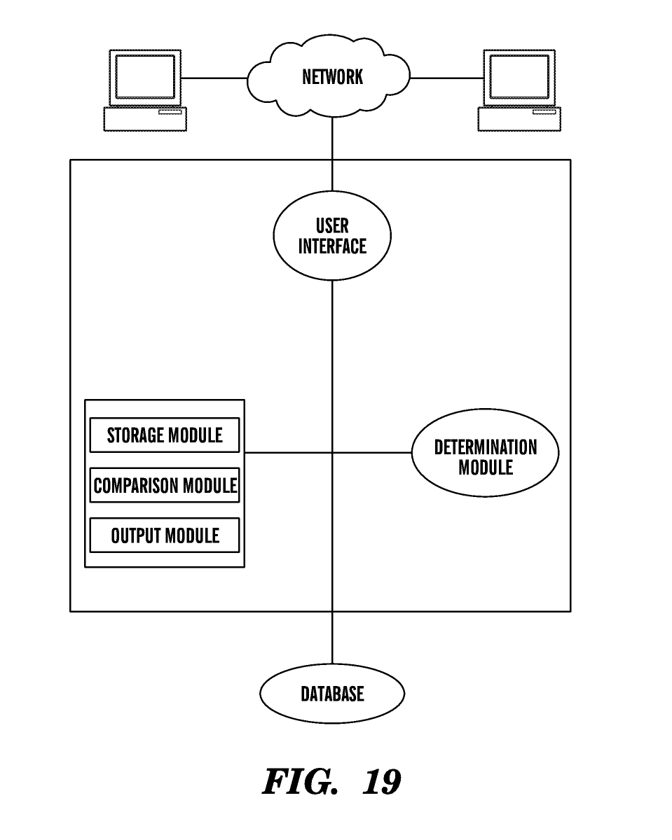

The computing and/or comparison module, or any other module of the invention, can include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). In some embodiments users can directly access data (via Hypertext links for example) residing on Internet databases using a HTML interface provided by Web browsers and Web servers (FIG. 19).

The computing and/or comparison module provides a computer readable comparison result that can be processed in computer readable form by predefined criteria, or criteria defined by a user, to provide content based in part on the comparison result that may be stored and output as requested by a user using an output module, e.g., a display module.

In some embodiments, the content displayed on the display module can be a report, e.g. the level of the expression products of one or more marker genes selected from Table 1 and/or Table 2 and/or the level of phosphorylated SMAD2/3 in the sample obtained from a subject. In some embodiments, the report can denote raw values of the level of expression products of one or more marker genes selected from Table 1 and/or Table 2 and/or the level of phosphorylated SMAD2/3 in the test sample or it indicates a percentage or fold increase as compared to a reference level, and/or provides a signal that the subject is or is not likely to benefit from cytoreductive surgery as described above herein.

In some embodiments, if the computing module determines that the level of expression products of one or more marker genes selected from Table 1 and/or Table 2 and/or the level of phosphorylated SMAD2/3 in the sample obtained from a subject is different by a statistically significant amount than the reference level, the display module provides a report displaying a signal indicating that the level in the sample obtained from a subject is different than that of the reference level. In some embodiments, the content displayed on the display module or report can be the relative level of expression products of one or more marker genes selected from Table 1 and/or Table 2 and/or the level of phosphorylated SMAD2/3 in the sample obtained from a subject as compared to the reference level. In some embodiments, the signal can indicate the degree to which the level of expression products of one or more marker genes selected from Table 1 and/or Table 2 and/or the level of phosphorylated SMAD2/3 in the sample obtained from the subject varies from the reference level. In some embodiments, the signal can indicate that the subject is likely or not likely to benefit from cytoreductive surgery. In some embodiments, the content displayed on the display module or report can be a numerical value indicating one of these risks or probabilities. In such embodiments, the probability can be expressed in percentages or a fraction. In some embodiments, the content displayed on the display module or report can be single word or phrases to qualitatively indicate a risk or probability. For example, a word “unlikely” can be used to indicate a lower likelihood of benefiting from cytoreductive surgery, while “likely” can be used to indicate a high likelihood of benefiting from cytoreductive surgery.

In one embodiment of the invention, the content based on the computing and/or comparison result is displayed on a computer monitor. In one embodiment of the invention, the content based on the computing and/or comparison result is displayed through printable media. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the computing/comparison result. It should be understood that other modules of the invention can be adapted to have a web browser interface. Through the Web browser, a user can construct requests for retrieving data from the computing/comparison module. Thus, the user will typically point and click to user interface elements such as buttons, pull down menus, scroll bars and the like conventionally employed in graphical user interfaces.

Systems and computer readable media described herein are merely illustrative embodiments of the invention for determining the level of expression products of one or more marker genes selected from Table 1 and/or Table 2 and/or the level of phosphorylated SMAD2/3 in a sample obtained from a subject, and therefore are not intended to limit the scope of the invention. Variations of the systems and computer readable media described herein are possible and are intended to fall within the scope of the invention. The modules of the machine, or those used in the computer readable medium, may assume numerous configurations. For example, function may be provided on a single machine or distributed over multiple machines.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, or “reduction”, are all used herein to mean a decrease by a statistically significant amount. In some embodiments, “reduce,” “reduction” or “decrease” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or does not encompass a complete inhibition or reduction as compared to a reference level.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of ovarian cancer. A subject can be female.

A subject can be one who has been previously diagnosed with or identified as suffering from or having a condition in need of treatment (e.g. cancer) or one or more complications related to such a condition, and optionally, have already undergone treatment for cancer or the one or more complications related to cancer. Alternatively, a subject can also be one who has not been previously diagnosed as having cancer or one or more complications related to cancer. For example, a subject can be one who exhibits one or more risk factors for cancer or one or more complications related to cancer or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. ovarian cancer. The term “treating” includes reducing or alleviating at least one adverse effect or symptom of a condition, disease or disorder associated with a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); Current Protocols in Protein Science (CPPS) (John E. Coligan, et. al., ed., John Wiley and Sons, Inc.), which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method of treatment comprising,
   detecting and/or measuring, in a sample obtained from a subject in need of treatment for ovarian cancer, the level of activation of at least one pathway selected from the group consisting of:
   TGF-β/Smad signaling; RTK/Ras/MAPK/Egr-1 signaling; AMPK/Egr-1 signaling; and Hedgehog/Gli signaling;
   administering cytoreductive surgery to the subject if the level of activation is not increased relative to a reference level; and
   not administering cytoreductive surgery to the subject if the level of activation is increased relative to a reference level.
2. A method of treatment comprising,
   detecting and/or measuring, in a sample obtained from a subject in need of treatment for ovarian cancer, the level of expression products of at least one marker gene selected from Table 1 or Table 2;
   or the level of phosphorylated SMAD2 or SMAD3;
   administering cytoreductive surgery to the subject if the level of expression products selected from Table 1 or the level of phosphorylated SMAD2 or SMAD3 are not increased relative to a reference level or the level of expression products selected from Table 2 are not decreased relative to a reference level; and
   not administering cytoreductive surgery to the subject if the level of expression products selected from Table 1 or the level of phosphorylated SMAD2 or SMAD3 are increased relative to a reference level or the level of expression products selected from Table 2 are decreased relative to a reference level.
3. A method of treatment comprising,
   administering cytoreductive surgery to a subject in need of treatment for ovarian cancer if the level of activation of at least one pathway selected from the group consisting of:
      TGF-β/Smad signaling; RTK/Ras/MAPK/Egr-1 signaling; AMPK/Egr-1 signaling; and Hedgehog/Gli signaling
   in a sample obtained from the subject is determined and/or measured not increased relative to a reference level; and
   not administering cytoreductive surgery to the subject in need of treatment for ovarian cancer if the level of activation of at least one pathway selected from the group consisting of:
      TGF-β/Smad signaling; RTK/Ras/MAPK/Egr-1 signaling; AMPK/Egr-1 signaling; and Hedgehog/Gli signaling
   in a sample obtained from the subject is determined and/or measured to be increased relative to a reference level.
4. A method of treatment comprising,
   administering cytoreductive surgery to a subject in need of treatment for ovarian cancer if the level of expression products selected from Table 1 or the level of phosphorylated SMAD2 or SMAD3 in a sample obtained from the subject is determined and/or measured to not be increased relative to a reference level or the level of expression products selected from Table 2 in a sample obtained from the subject is determined and/or measured to not be decreased relative to a reference level; and
   not administering cytoreductive surgery to a subject in need of treatment for ovarian cancer if the level of expression products selected from Table 1 or the level of phosphorylated SMAD2 or SMAD3 in a sample obtained from the subject is determined and/or measured to be increased relative to a reference level or the level of expression products selected from Table 2 in a sample obtained from the subject is determined and/or measured to be decreased relative to a reference level.
5. The method of paragraph 2 or 4, wherein the one or more marker genes is selected from the group consisting of:
   MMP2, TIMP3, ADAMTS1, VCL, TGFB1, SPARC, CYR61; EGR1, SMADs; GLIs, VCAN, CNY61, LOX, TAFs, ACTA2, POSTN, CXCL14, CCL13, FAP, NUAK1, PTCH1, TGFBR2; and TNFAIP6.
6. The method of paragraph 2 or 4, wherein the one or more marker genes is selected from the group consisting of:
   POSTN, CXCL14, CCL13, FAP, NUAK1, PTCH1, TGFBR2; and TNFAIP6.
7. The method of paragraph 6, wherein the level of the expression products of POSTN, CXCL14, CCL13, FAP, NUAK1, PTCH1, TGFBR2; and TNFAIP6 is determined
8. The method of any of paragraphs 1-7, wherein the expression products are mRNA expression products.
9. The method of any of paragraphs 1-8, wherein the expression products are polypeptide expression products.
10. The method of any of paragraphs 1-9, wherein the subject has advanced stage ovarian cancer.
11. The method of any of paragraphs 1-10, wherein the sample is a tumor cell sample.
12. The method of any of paragraphs 1-11, wherein the subject is a human.
13. An assay comprising, detecting and/or measuring, in a sample obtained from a subject in need of treatment for ovarian cancer, the level of activation of at least one pathway selected from the group consisting of:
   TGF-β/Smad signaling; RTK/Ras/MAPK/Egr-1 signaling; AMPK/Egr-1 signaling; and Hedgehog/Gli signaling;
   wherein the subject is likely to benefit from cytoreductive surgery if the level of activation is not increased relative to a reference level; and
   wherein the subject is not likely to benefit from cytoreductive surgery if the level of activation is increased relative to a reference level.
14. An assay comprising, detecting and/or measuring, in a sample obtained from a subject in need of treatment for ovarian cancer, the level of expression products of at least one marker gene selected from Table 1 or Table 2;
   or the level of phosphorylated SMAD2 or SMAD3;
   wherein the subject is likely to benefit from cytoreductive surgery if the level of expression products selected from Table 1 or the level of phosphorylated SMAD2 or SMAD3 are not increased relative to a reference level or the level of expression products selected from Table 2 are not decreased relative to a reference level; and wherein the subject is not likely to benefit from cytoreductive surgery if the level of expression products selected from Table 1 or the level of phosphorylated SMAD2 or SMAD3 are increased relative to a reference level or the level of expression products selected from Table 2 are decreased relative to a reference level.
15. The assay of paragraph 14, wherein the one or more marker genes is selected from the group consisting of:
   MMP2, TIMP3, ADAMTS1, VCL, TGFB1, SPARC, CYR61; EGR1, SMADs; GLIs, VCAN, CNY61, LOX, TAFs, ACTA2, POSTN, CXCL14, CCL13, FAP, NUAK1, PTCH1, TGFBR2; and TNFAIP6.
16. The assay of paragraph 14, wherein the one or more marker genes is selected from the group consisting of:
   POSTN, CXCL14, CCL13, FAP, NUAK1, PTCH1, TGFBR2; and TNFAIP6.
17. The assay of paragraph 16, wherein the level of the expression products of POSTN, CXCL14, CCL13, FAP, NUAK1, PTCH1, TGFBR2; and TNFAIP6 is determined and/or measured.
18. The assay of any of paragraphs 13-17, wherein the expression products are mRNA expression products.
19. The assay of any of paragraphs 13-17, wherein the expression products are polypeptide expression products.
20. The assay of any of paragraphs 13-19, wherein the level of expression of a marker gene product is determined and/or measured using an method selected from the group consisting of:
   RT-PCR; quantitative RT-PCR; Northern blot; microarray based expression analysis; Western blot; immunoprecipitation; enzyme-linked immunosorbent assay (ELISA); radioimmunological assay (RIA); sandwich assay; fluorescence in situ hybridization (FISH); immunohistological staining; radioimmunometric assay; immunofluoresence assay; mass spectroscopy and immunoelectrophoresis assay.
21. The assay of any of paragraphs 13-19, wherein the subject has advanced stage ovarian cancer.
22. The assay of any of paragraphs 13-21, wherein the sample is a tumor cell sample.
23. The assay of any of paragraphs 13-22, wherein the subject is a human.
24. A method of determining if a subject is likely to benefit from cytoreductive surgery, the method comprising, detecting and/or measuring, in a sample obtained from a subject in need of treatment for ovarian cancer, the level of activation of at least one pathway selected from the group consisting of:
   TGF-β/Smad signaling; RTK/Ras/MAPK/Egr-1 signaling; AMPK/Egr-1 signaling; and Hedgehog/Gli signaling;
   wherein the subject is likely to benefit from cytoreductive surgery if the level of activation is not increased relative to a reference level; and
   wherein the subject is not likely to benefit from cytoreductive surgery if the level of activation is increased relative to a reference level.
25. A method of determining if a subject is likely to benefit from cytoreductive surgery, the method comprising, detecting and/or measuring, in a sample obtained from a subject in need of treatment for ovarian cancer, the level of expression products of at least one marker gene selected from Table 1 or Table 2;
   or the level of phosphorylated SMAD2 or SMAD3;
   wherein the subject is likely to benefit from cytoreductive surgery if the level of expression products selected from Table 1 or the level of phosphorylated SMAD2 or SMAD3 are not increased relative to a reference level or the level of expression products selected from Table 2 are not decreased relative to a reference level; and
   wherein the subject is not likely to benefit from cytoreductive surgery if the level of expression products selected from Table 1 or the level of phosphorylated SMAD2 or SMAD3 are increased relative to a reference level or the level of expression products selected from Table 2 are decreased relative to a reference level.
26. A method of selecting a treatment regimen for a subject with ovarian cancer, the method comprising, detecting and/or measuring, in a sample obtained from a subject in need of treatment for ovarian cancer, the level of activation of at least one pathway selected from the group consisting of:
   TGF-β/Smad signaling; RTK/Ras/MAPK/Egr-1 signaling; AMPK/Egr-1 signaling; and Hedgehog/Gli signaling;
   selecting a treatment regimen comprising cytoreductive surgery if the level of activation is not increased relative to a reference level; and
   selecting a treatment regimen not comprising cytoreductive surgery if the level of activation is increased relative to a reference level.
27. A method of selecting a treatment regimen for a subject with ovarian cancer, the method comprising, detecting and/or measuring, in a sample obtained from a subject in need of treatment for ovarian cancer, the level of expression products of at least one marker gene selected from Table 1 or Table 2;
   or the level of phosphorylated SMAD2 or SMAD3;
   selecting a treatment regimen comprising cytoreductive surgery if the level of expression products selected from Table 1 or the level of phosphorylated SMAD2 or SMAD3 are not increased relative to a reference level or the level of expression products selected from Table 2 are not decreased relative to a reference level; and
   selecting a treatment regimen not comprising cytoreductive surgery if the level of expression products selected from Table 1 or the level of phosphorylated SMAD2 or SMAD3 are increased relative to a reference level or the level of expression products selected from Table 2 are decreased relative to a reference level.
28. The method of paragraphs 25 or 27, wherein the one or more marker genes is selected from the group consisting of:
   MMP2, TIMP3, ADAMTS1, VCL, TGFB1, SPARC, CYR61; EGR1, SMADs; GLIs, VCAN, CNY61, LOX, TAFs, ACTA2, POSTN, CXCL14, CCL13, FAP, NUAK1, PTCH1, TGFBR2; and TNFAIP6.
29. The method of paragraph 28, wherein the one or more marker genes is selected from the group consisting of:
   POSTN, CXCL14, CCL13, FAP, NUAK1, PTCH1, TGFBR2; and TNFAIP6.
30. The method of paragraph 28, wherein the level of the expression products of POSTN, CXCL14, CCL13, FAP, NUAK1, PTCH1, TGFBR2; and TNFAIP6 is determined
31. The method of any of paragraphs 24-30, wherein the expression products are mRNA expression products.
32. The method of any of paragraphs 24-30, wherein the expression products are polypeptide expression products.
33. The method of any of paragraphs 24-32, wherein the level of expression of a marker gene product is determined and/or measured using an method selected from the group consisting of:
   RT-PCR; quantitative RT-PCR; Northern blot; microarray based expression analysis; Western blot; immunoprecipitation; enzyme-linked immunosorbent assay (ELISA); radioimmunological assay (RIA); sandwich assay; fluorescence in situ hybridization (FISH); immunohistological staining; radioimmunometric assay; immunofluoresence assay; mass spectroscopy and immunoelectrophoresis assay.
34. The method of any of paragraphs 24-33, wherein the subject has advanced stage ovarian cancer.
35. The method of any of paragraphs 24-34, wherein the sample is a tumor cell sample.
36. The method of any of paragraphs 24-35, wherein the subject is a human.
37. The method or assay of any of paragraphs 24-36, further comprising administering cytoreductive surgery to the subject if the level of activation is not increased relative to a reference level; and not administering cytoreductive surgery to the subject if the level of activation is increased relative to a reference level.
38. The method of any of paragraphs 1-37, further comprising a step of comparing the level of expression and/or phosphorylation determined and/or measured in the sample obtained from the subject with a reference level.
39. A computer system for determining if subject in need of treatment for ovarian cancer is likely to benefit from cytoreductive surgery, the system comprising:
   a measuring module configured to measure the level of activation of at least one pathway selected from the group consisting of:
      TGF-β/Smad signaling; RTK/Ras/MAPK/Egr-1 signaling; AMPK/Egr-1 signaling; and Hedgehog/Gli signaling;
   in a test sample obtained from a subject;
   a storage module configured to store output data from the measuring module;
   a comparison module adapted to compare the data stored on the storage module with a reference level, and to provide a retrieved content, and
   a display module for displaying whether the level in a test sample obtained from a subject is greater, by a statistically significant amount, than the reference level.
40. A computer system for determining if subject in need of treatment for ovarian cancer is likely to benefit from cytoreductive surgery, the system comprising:
   a measuring module configured to measure the level of expression products of at least one marker gene selected from Table 1 or Table 2;
      or the level of phosphorylated SMAD2 or SMAD3;
   in a test sample obtained from a subject;
   a storage module configured to store output data from the measuring module;
   a comparison module adapted to compare the data stored on the storage module with a reference level, and to provide a retrieved content, and
   a display module for displaying whether the level in a test sample obtained from a subject differs, by a statistically significant amount, from the reference level;
   wherein the subject is likely to benefit from cytoreductive surgery if the level of expression products selected from Table 1 or the level of phosphorylated SMAD2 or SMAD3 are not increased relative to a reference level or the level of expression products selected from Table 2 are not decreased relative to a reference level; and
   wherein the subject is not likely to benefit from cytoreductive surgery if the level of expression products selected from Table 1 or the level of phosphorylated SMAD2 or SMAD3 are increased relative to a reference level or the level of expression products selected from Table 2 are decreased relative to a reference level.
41. The system of paragraph 40, wherein the one or more marker genes is selected from the group consisting of:
   MMP2, TIMP3, ADAMTS1, VCL, TGFB1, SPARC, CYR61; EGR1, SMADs; GLIs, VCAN, CNY61, LOX, TAFs, ACTA2, POSTN, CXCL14, CCL13, FAP, NUAK1, PTCH1, TGFBR2; and TNFAIP6.
42. The method of paragraph 40, wherein the one or more marker genes is selected from the group consisting of:
   POSTN, CXCL14, CCL13, FAP, NUAK1, PTCH1, TGFBR2; and TNFAIP6.
43. The system of paragraph 40, wherein the level of the expression products of POSTN, CXCL14, CCL13, FAP, NUAK1, PTCH1, TGFBR2; and TNFAIP6 is determined
44. The system of any of paragraphs 39-43, wherein if the computing module determines that the level of of the marker in the test sample obtained from a subject is differs by a statistically significant amount from the reference level, the display module displays a signal indicating that the level in the sample obtained from a subject differs from the reference level.
45. The system of any of paragraphs 39-44, wherein the signal indicates whether the the subject is likely to benefit from cytoreductive surgery.
46. The system of any of paragraphs 39-45, further comprising creating a report based on the level of the marker.

### EXAMPLES

### Example 1

### Risk Prediction for Late-Stage Ovarian Cancer by Meta-Analysis of 1,622 Patient Samples: Biologic and Clinical Correlations

Ovarian cancer causes over 15,000 deaths per year in the United States, the majority of which present as advanced stage high grade, serous tumors. The survival of these patients is quite heterogeneous, and inaccurate prognosis would help with the clinical management of these patients. Published microarray-based prognostic gene signatures are however not yet sufficiently robust to employ clinically.

Described herein is the development and validation of a gene expression signature of survival for advanced stage serous ovarian cancer, integrating 13 publicly available datasets totaling 1,622 subjects. This signature was further tested on early stage serous disease. A second signature was developed for predicting debulking status. Prediction models were trained using a meta-analysis variation on the Compound Covariate method, tested via a “leave-one-dataset-out” procedure, and validation performed in additional datasets not meeting the selection criteria for training data. Selected genes from the debulking signature were validated by immunohistochemistry and qRT-PCR in two independent cohorts of 179 and 78 patients, respectively.

These signatures stratified patients into high- and low-risk groups (HR=2.17; 95% CI, 1.83 to 2.57) significantly better than the TCGA signature (P=0.016) and predict surgical outcome with a high sensitivity and specificity (AUC). POSTN, CXCL14, CCL13, FAP, NUAK1, PTCH1, and TGFBR2 were validated by qRT-PCR (P<0.05) and POSTN, CXCL14 and phosphorylated Smad2/3 by immunohistochemistry (P<0.0001) as independent predictors of debulking status. The sum of IHC intensities for these three proteins provided a tool that classified 93% of samples in the high and low risk groups for suboptimal debulking correctly, with an AUC of 0.89 (95% CI 0.84-0.93).

The signatures described herein provide the most accurate and well-validated prognostic models for early and advanced stage high-grade serous ovarian cancer and prediction of surgical debulking outcome.

### Introduction

Ovarian cancer is the most lethal gynecologic malignancy, causing over 15,000 deaths per year in the United States [1]. Advanced ovarian cancer (stages III and IV) accounted for the majority of the estimated 22,000 new cases of epithelial ovarian cancer in 2012 in the United States [1]. While significant improvements have been made in the median survival of women with advanced stage ovarian cancer, overall survival has essentially not changed during the last decades. For instance, although 80% of advanced stage papillary serous ovarian cancers initially respond to primary treatment with surgery and chemotherapy [2], most of them recur and eventually develop a drug-resistant phenotype. Reliable methods of stratification could group patients by response to initial therapy or survival time. There is a critical need for such classifiers to identify patients for recruitment into clinical trials as well as to identify novel targets for therapeutic intervention.

The inventors have developed the largest collection of ovarian cancer gene expression data to date [3], allowing them to systematically evaluate a range of previously published prognostic signatures [4-14]. The signature developed by The Cancer Genome Atlas (TCGA) consortium was previously the best available prognostic model [15]; however, even this signature is insufficiently accurate for clinical application.

As described herein the inventors used a meta-analytic approach to leverage more than 1,600 publicly available, clinically-annotated microarray assays of high-grade, primary serous tumors to comprehensively address two important objectives for prognostication of ovarian cancer. These are to (i) develop a prognostic gene signature for overall survival of early and late-stage patients and (ii) predict suboptimal cytoreductive surgery. The inventors performed extensive signature evaluation demonstrating significant improvement over existing signatures. Furthermore, this work establishes the existence of a signature predictive for suboptimal cytoreduction providing for the avoidance of unsuccessful surgery through a genomic or immunohistochemical test at diagnosis. These results provide for more personalized treatment for women with ovarian cancer.

### Methods

Dataset and Patient Eligibility Criteria:

Gene expression data are available as processed, normalized datasets in the inventors' ovarian cancer microarray database [17]. Criteria for inclusion of cohorts in this database, the corresponding literature reviews, and the data processing protocols have been described previously[17]. The meta-analysis described herein is restricted to primary, late-stage (stage III or IV), high-grade, serous tumorswith available overall survival time-to-event data (Table 3). For prediction model training, a minimum sample size of 75 for survival prediction and 50 for binary classification was required. Only datasets published before March 2012 were considered in the training phase. Datasets that did not pass these two additional training criteria were used as validation data for the final model. The final model was tested on the 30 early-stage, high-grade samples from TCGA; these samples contained 8 patients with events.

Clinical Endpoints:

The primary endpoint was overall survival (OS) from initial diagnosis to death. Suboptimal debulking was defined as presence of macroscopic residual tumor in two public datasets [7, 18], otherwise as residual tumor mass >1 cm.

Model Training:

For the model training approach, as many publically available datasets as possible were used to identify robust good and bad prognosis genes, i.e., genes for which up-regulation was consistently associated with longer and shorter survival, respectively. To test for association with survival, the univariate Cox coefficients and their standard errors were calculated for each gene in all datasets. To summarize the Cox coefficients of a gene i across training datasets into a single coefficient βᵢ, these coefficients were pooled in a fixed effects meta-analysis [19]. Genes with a pooled Cox coefficient significant larger than 0 represent bad prognosis genes, while those with a Cox coefficient significantly smaller than 0 are good prognosis genes. In the model training step, genes were ranked by p-value against the null hypothesis of pooled coefficient equal to 0. Then, a set number m of the top-ranked genes were used in the prediction model. This model can be used to calculate a risk score of patient j, rⱼ, with the meta-analysis variant of the compound covariate score [20]. In this approach, the expression of gene i in subject j, xᵢⱼ, is weighted by the pooled Cox coefficient of gene i (βᵢ) to calculate the risk score r_{J}:

$${r}_{j} = {\sum }_{i = 1}^{m} {β}_{i} {x}_{ij}$$

This linear predictor represents a weighted average of the expression of genes in the signature. To ensure that gene expression measurements are on the same scale across studies, all datasets were centered to zero mean and scaled to unit variance. For binary classification, we proceeded analogously, pooling coefficients of a univariate logistic regression model for each gene.

Validation Metrics:

Gene signatures were evaluated by Hazard Ratio (HR) of dichotomized patient risk scores. Dichotomization cutoffs corresponded to the medians of these risk scores in the training cohorts. Significance of HR differences between signatures was estimated by bootstrap. Binary outcome classifiers were evaluated by area under the Receiver Operating Characteristic (ROC) curve (AUC). Multivariate models included clinicopathologic or demographic characteristics as predictors, along with the meta-analysis risk stratifications. These models were 5-fold cross-validated. Details of the implementations of published models [7, 16, 21] are described herein.

Quantitative RT-PCR:

Quantitative RT-PCR (qRT-PCR) was performed as previously described [11] on 20 ng amplified RNA from 39 suboptimally and 39 optimally debulked specimens selected randomly from the Bonome et al. cohort [6] that had not been used in model training. As tumor stage is associated with debulking status [22], numbers of stage III and IV patients (31 and 8, respectively) in the optimal and suboptimal groups were balanced to disassociate stage and debulking status in the qRT-PCR validation cohort. Primer sets were selected (Table 4) for housekeeping genes GAPDH, GUSB, and ACTB and 8 genes showing highly differentiated expression levels through the meta-analysis.

Immunohistochemistry (IHC):

Immunohistochemical staining of POSTN (BioVendor R&D, 1.25 μg/mL), CXCL14 (Abcam, ab46010, 2.5 μg/mL) and pSmad2/3 (Cell Signaling, #3101, recognizing only phosphorylated Smad2 or Smad3 by TGF-β receptor, 1:200) was done on an independent validation tissue microarray consisting of 216 stage III/IV high-grade serous ovarian cancers obtained from patients with informed consent at the Massachusetts General Hospital (MGH) between 1993 and 2009. Debulking status was available for 179 cancers (136 optimal and 43 suboptimal). Deparaffinized sections were subjected to antigen retrieval (citrate buffer, pH=6.1, 95° C. 30 min), incubated with each primary antibody at room temperature for 45 min, visualized with ImmPRESS Peroxidase Polymer Detection Reagents (Vector Laboratories) and 3,3′-diaminobenzidine (DAB), and counterstained with Mayer's hematoxylin. Intensity scores were calculated as the average difference in staining intensity between the tumor and stroma areas [23].

Multivariate qRT-PCR and IHC Models:

The signs of the coefficients from the microarray-based signature were used, i.e., all genes were equally weighted, with the expression levels of down-regulated genes in suboptimal subtracted from the ones of up-regulated genes. Group sizes for patient stratification in high-, medium- and low-risk corresponded to the numbers of suboptimal and optimal tumors for high- and low-risk, respectively. The 33% of high-risk samples with lowest risk and the 33% of low-risk samples with highest risk were then classified as medium-risk and were excluded from the accuracy calculation.

### Results

A rigorous meta-analysis approach was used to publicly available high grade, advanced stage serous ovarian cancer microarray gene expression profiles (Table 3) [3]. This included the training and validation of prognostic signatures that stratify high- and low-risk patients with early-stage and late-stage serous ovarian cancer. An additional signature was used to identify advanced stage serous tumors that cannot be optimally debulked to ≦1 cm of residual tumor.

Overall Survival Gene Signature Stratifies Patients in High- and Low-Risk Groups.

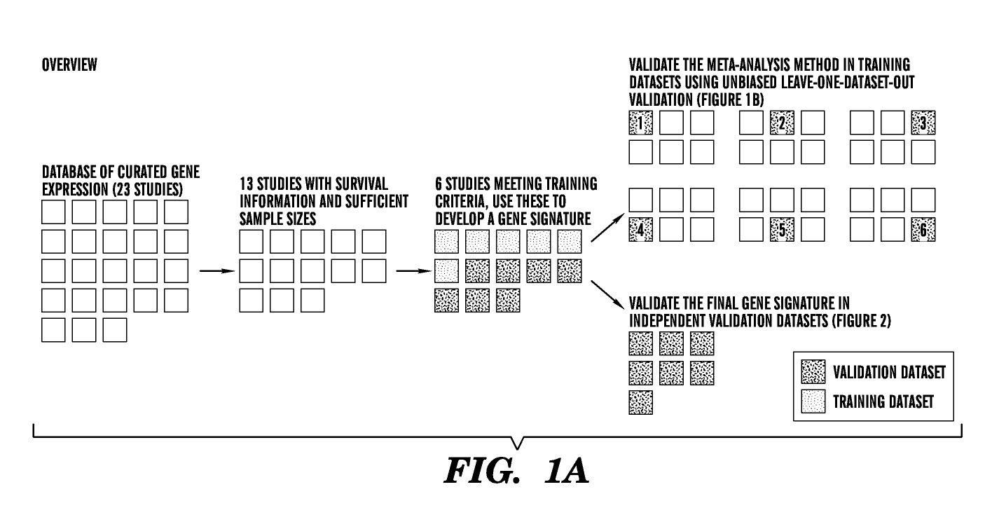

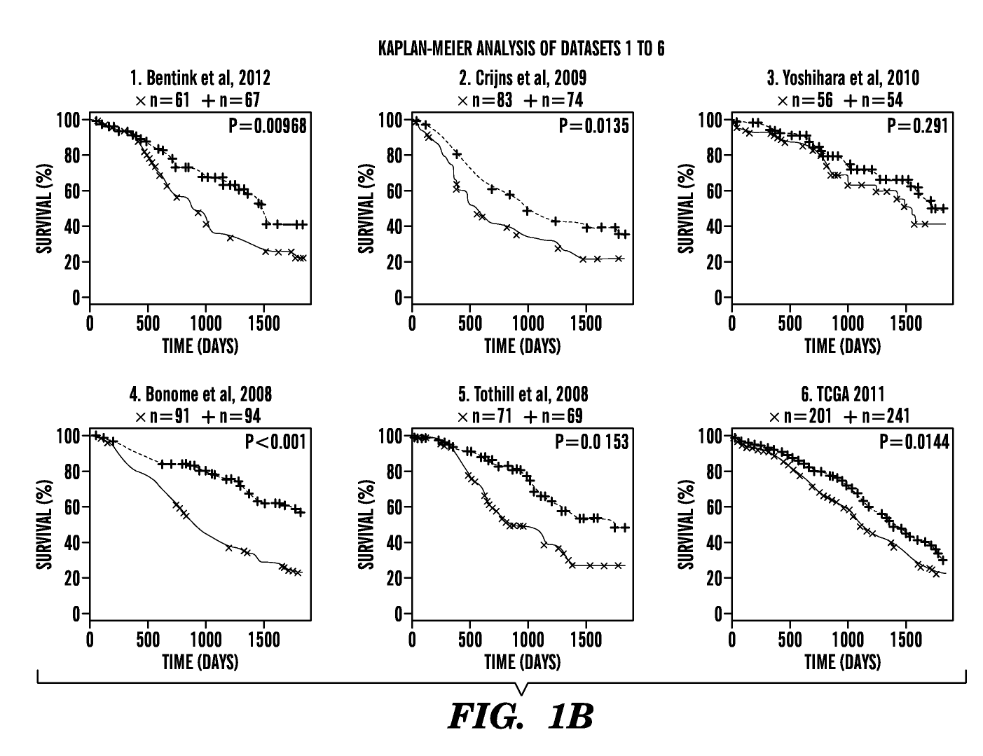

A new gene signature was identified, consisting of genes whose expression displayed the most significant association with overall survival across major public datasets (see Methods). The meta-analysis allowed a simple approach to use these datasets for both training and validation, and similar to classic cross-validation to validate a prediction model the remaining datasets for training. This approach provides an unbiased estimate of the prediction performance, since the training data is never used for validation (FIG. 1A).

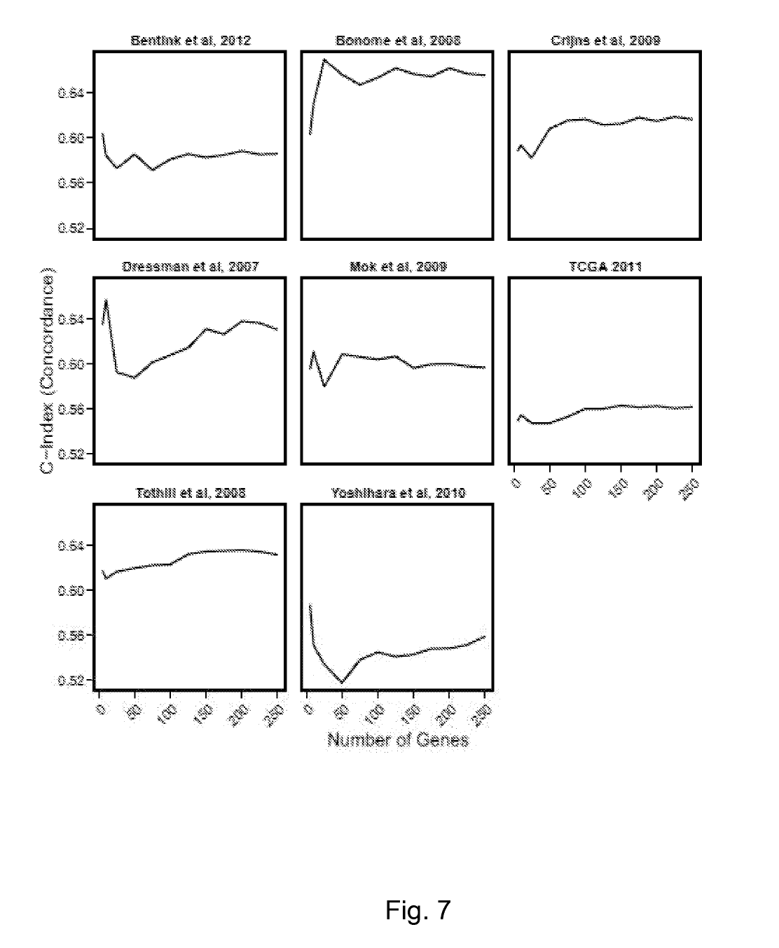

A standard signature size of 200 genes was used for all models, a size sufficiently small to be practically useful in a clinical test. In general, the size of the gene signature had only a modest impact on the prediction accuracy in the algorithm, as long as the signature size was larger than 100 genes (FIG. 7). Significant risk stratification was found in all but the Yoshihara et al. 2010 dataset [13] (FIG. 1B).

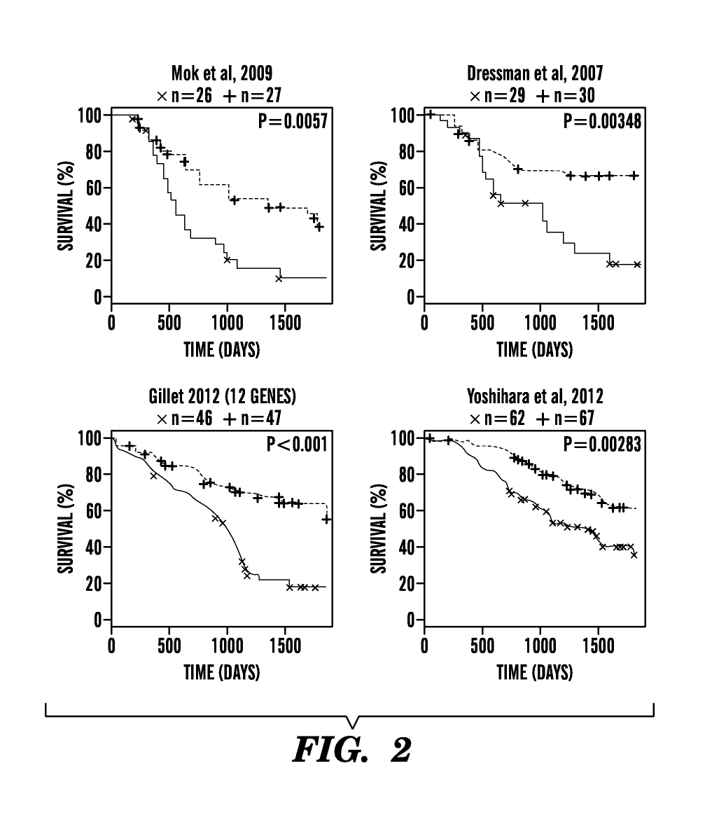

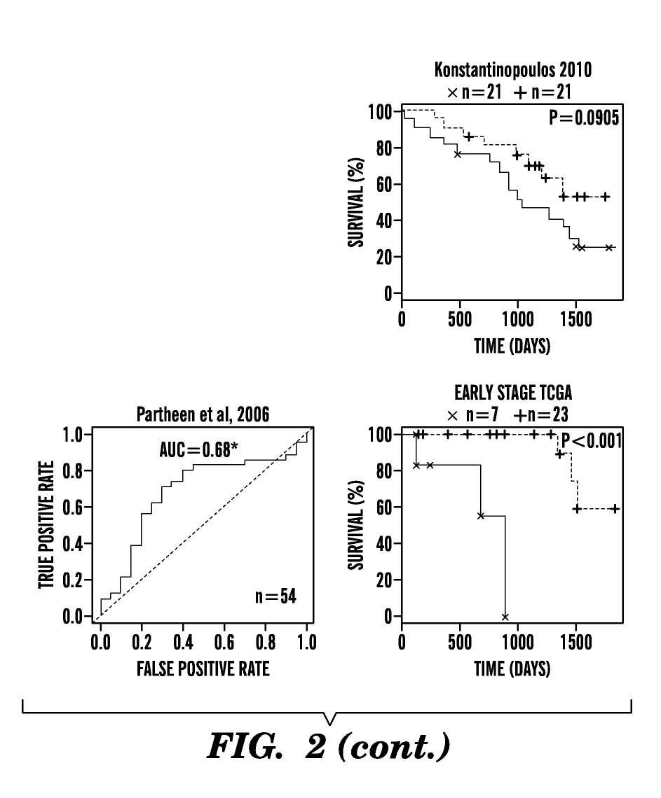

The signature was tested in 7 additional datasets not meeting the criteria for training datasets. These included a qRT-PCR dataset [24], three datasets not passing the minimum training sample size of 75 [11, 25, 26], a dataset which became available after the model was finalized [14], the TCGA early-stage, high-grade samples [7], and a dataset for which survival was annotated with a binary label rather than time to death [18]. The model validated in all of these datasets as shown by Kaplan-Meier analysis and Receiver Operating Characteristic plots (FIG. 2).

The Survival Signature Outperforms Existing Prognostic Factors and Gene Signatures.

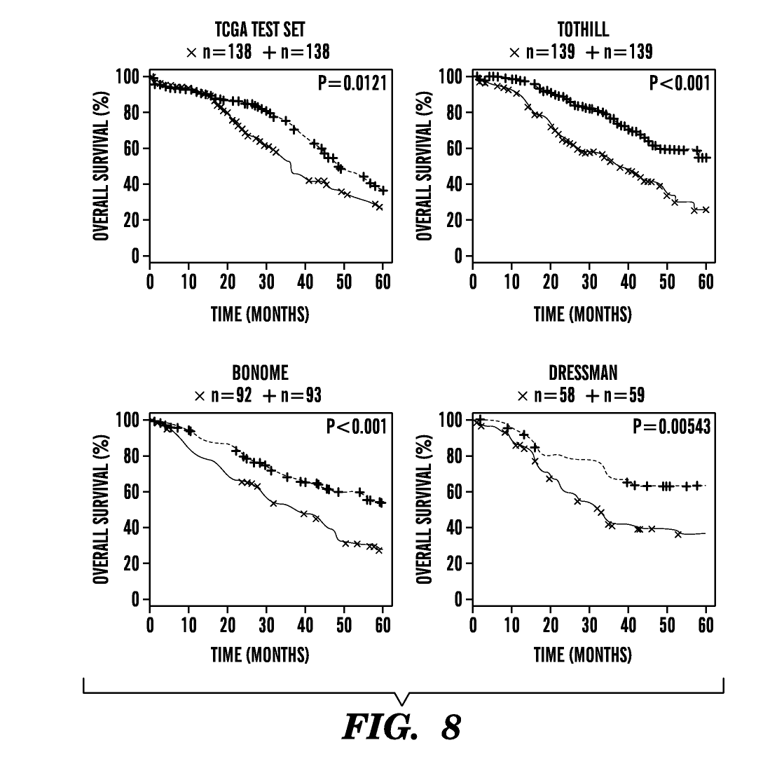

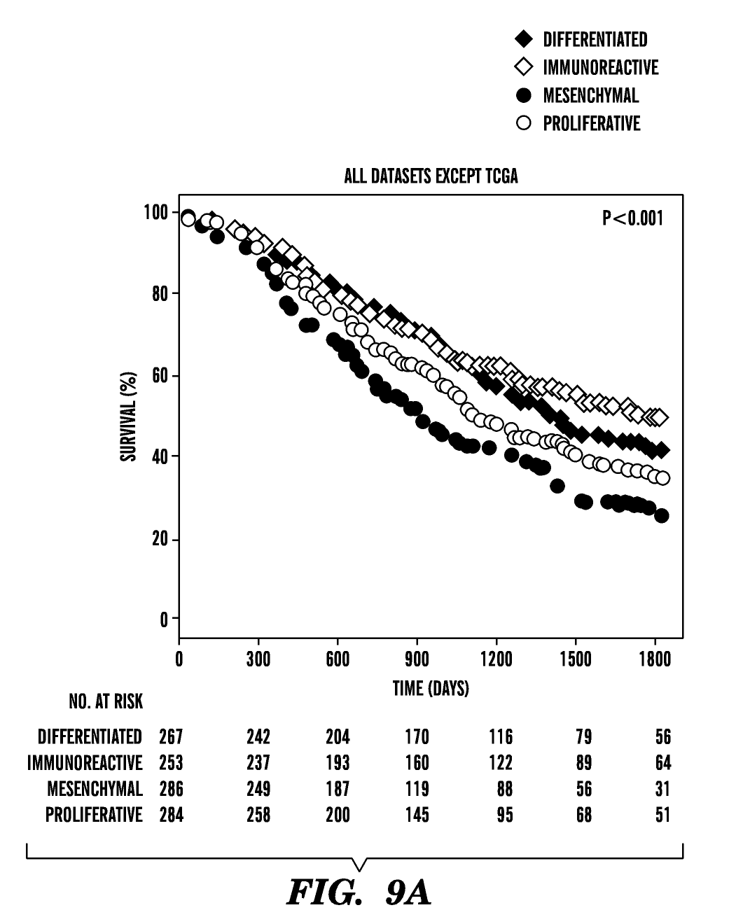

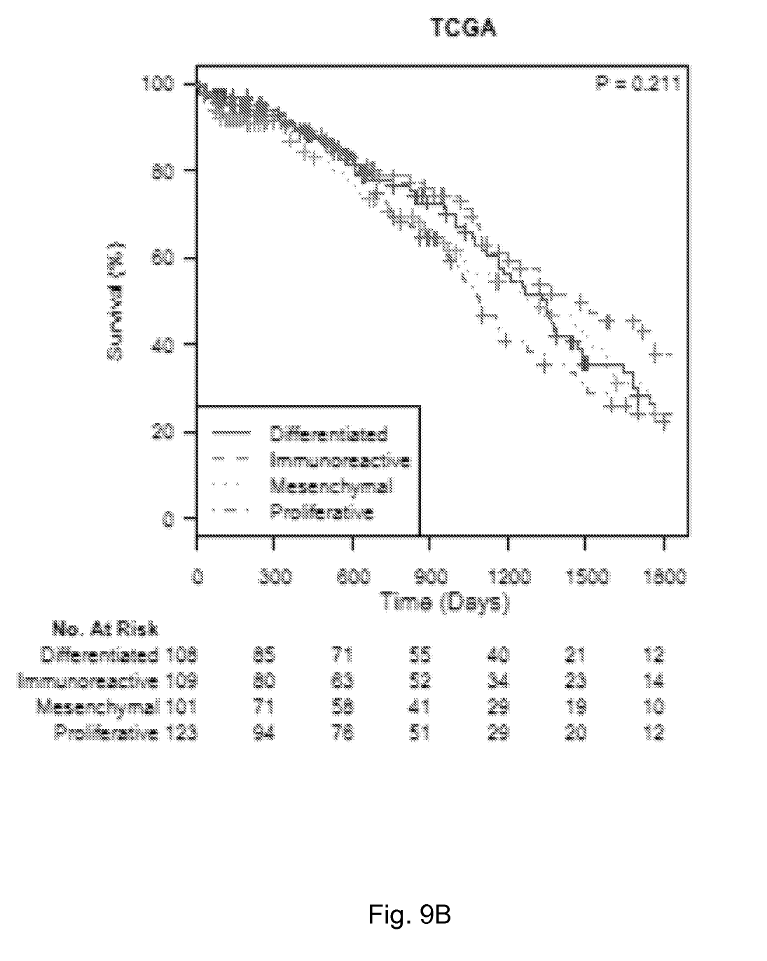

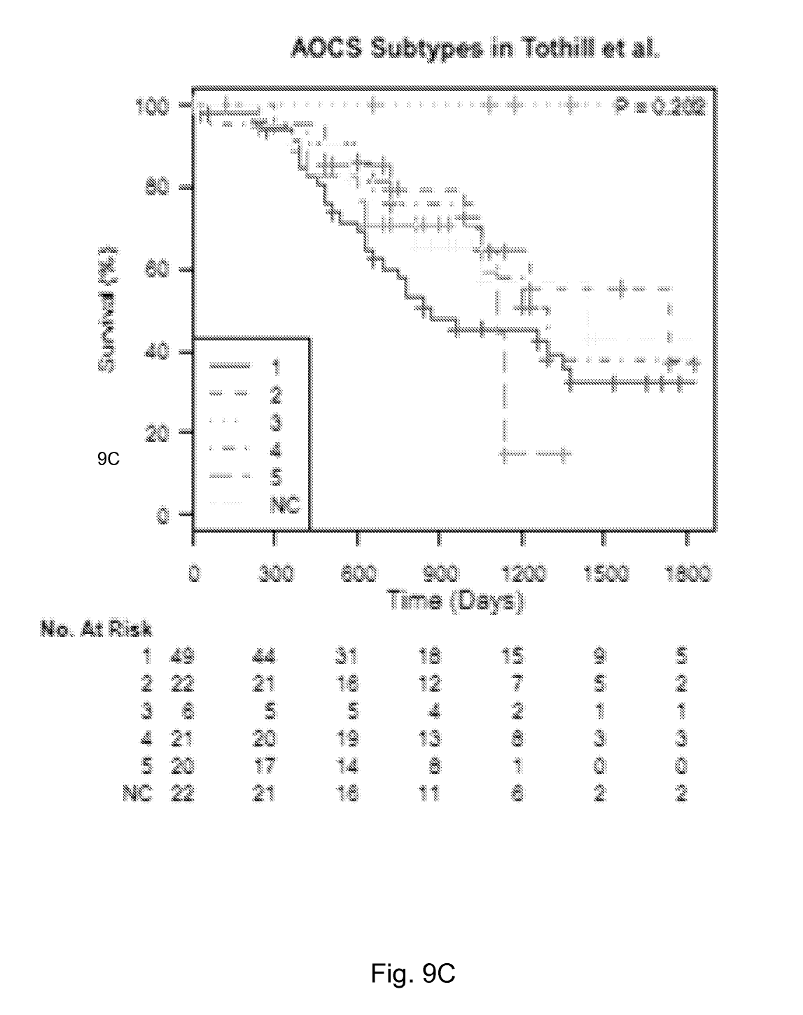

Kaplan-Meier stratifications from clinical prognostic factors, from the original TCGA gene signature, (FIG. 8, [7]) the best signature published before July 2012 [15], and from the more recent TCGA signature developed by the same investigators (FIGS. 9A-9C) were compared. Clinical prognostic factors include optimal debulking [6], age and tumor stage at diagnosis. All three factors were only available for 4 datasets; however, both stage and debulking status were available for 7 datasets (Table 3). These two factors were therefore focused on.

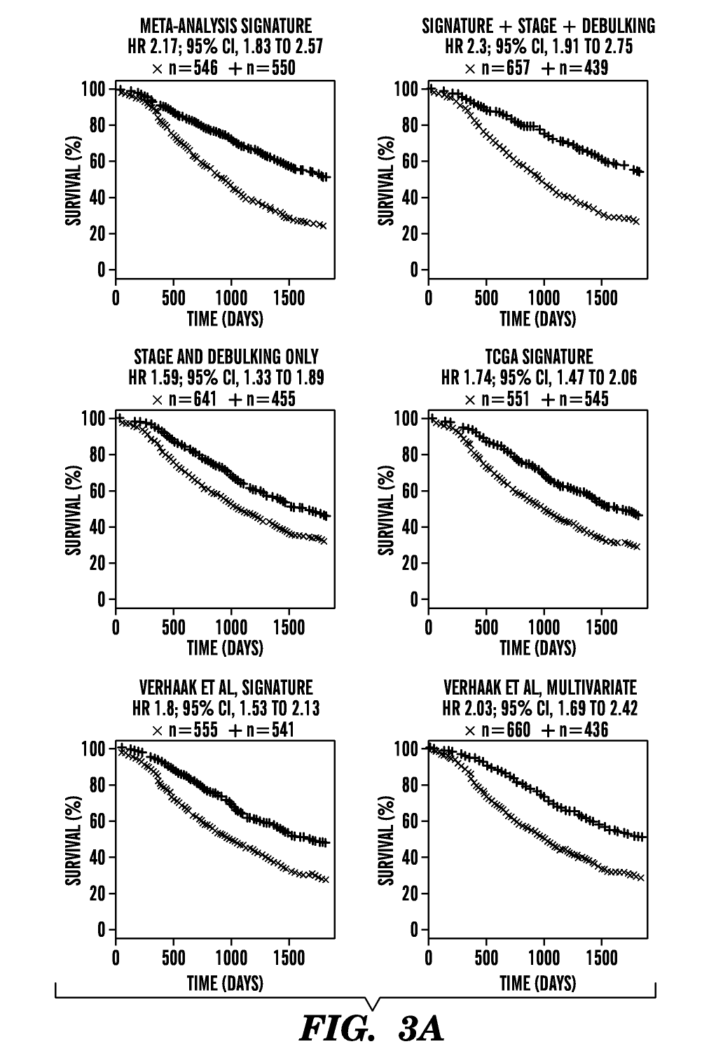

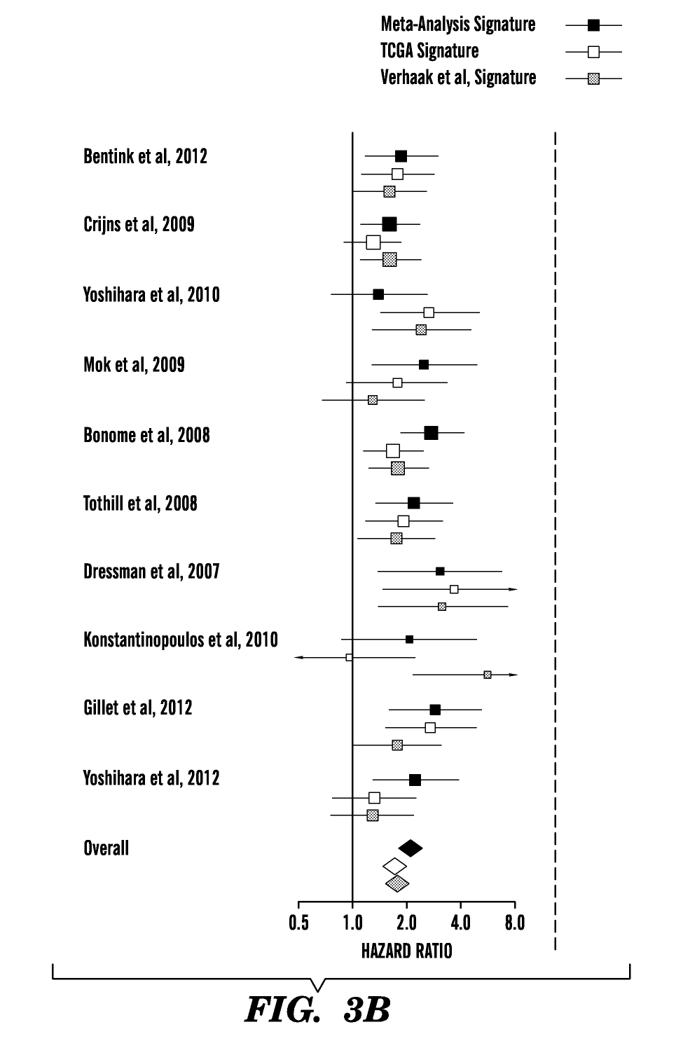

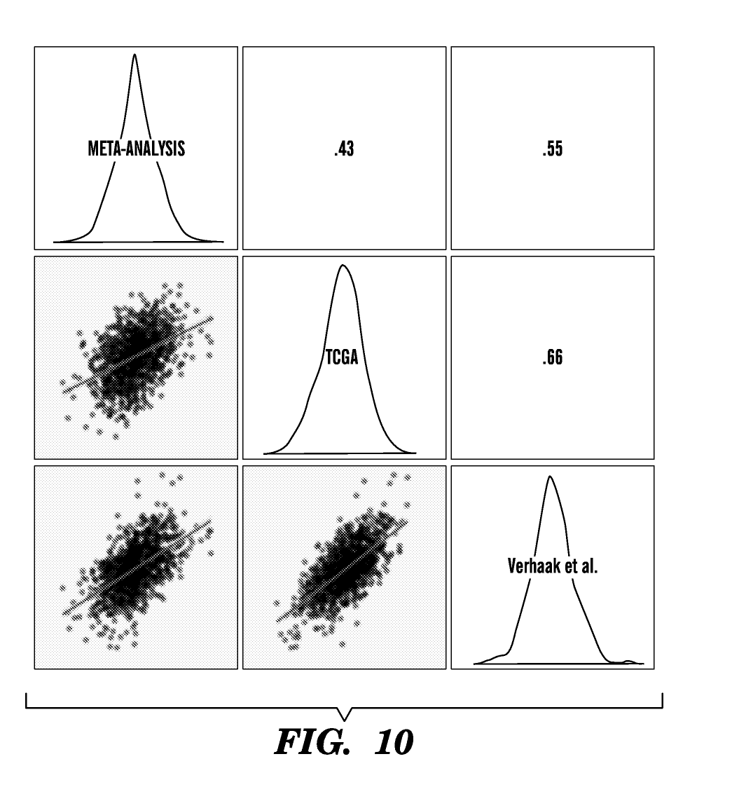

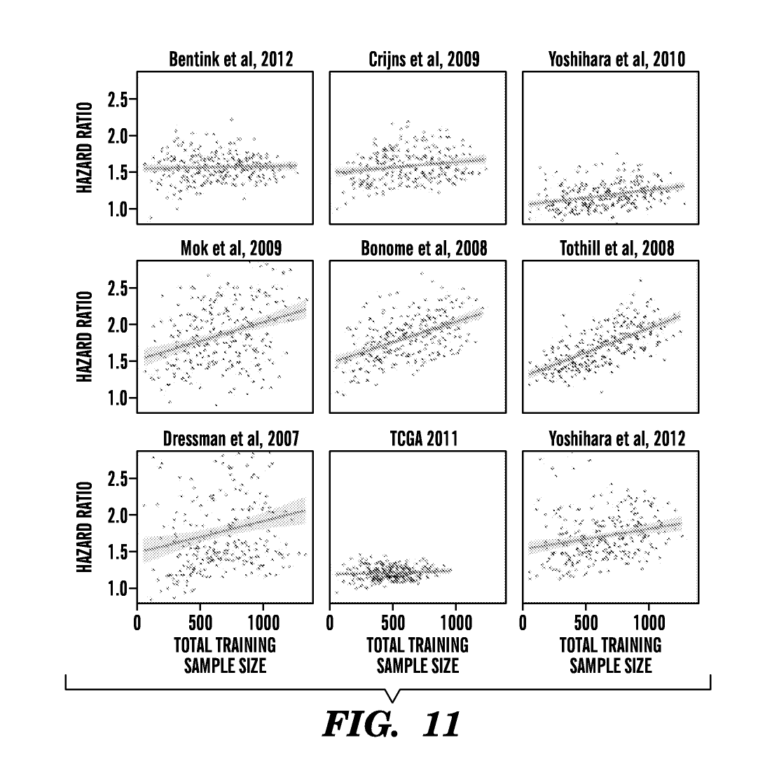

Patient stratification using the signature described herein was superior to clinical factors or either TCGA gene signatures (FIG. 3A-3B). Over all cohorts excluding TCGA (1,096 patients where direct comparison to the TCGA signatures could be made), patients classified as high-risk by the signature described herein had a median survival of 30.5 months (95% CI 27.6-33.5) compared to 61.1 months (95% CI 55.2-68.0, HR=2.17; 95% CI for HR 1.83-2.57) for the low-risk patients. Addition of stage and debulking status to this signature in a multivariate model yielded an improved HR of 2.3 (95% CI 1.91-2.75). The present signature alone provided an overall increase in HR of 0.43 (95% CI 0.04-0.82) compared to the original TCGA signature. The two signatures proposed by the TCGA consortium performed very similarly compared to each other (HR difference 0.06; 95% CI−0.26-0.38, FIGS. 3A-3B). Finally, it was shown that the meta-analysis training method described herein is superior to single study training (FIG. 10).

A Novel Signature Predicts Suboptimal Debulking Surgery.

Repeating the meta-analysis approach, a gene signature for predicting unsuccessful debulking surgery was developed (Tables 1 and 2). This signature was expected to be different from the survival signature, since the biologic basis for optimal surgical removal of tumor tissue and patient survival is likely not the same. We All 8 late-stage microarray datasets with available debulking information were utilized (Table 3), excluding half of the Bonome samples (n=79) for validation by qRT-PCR.

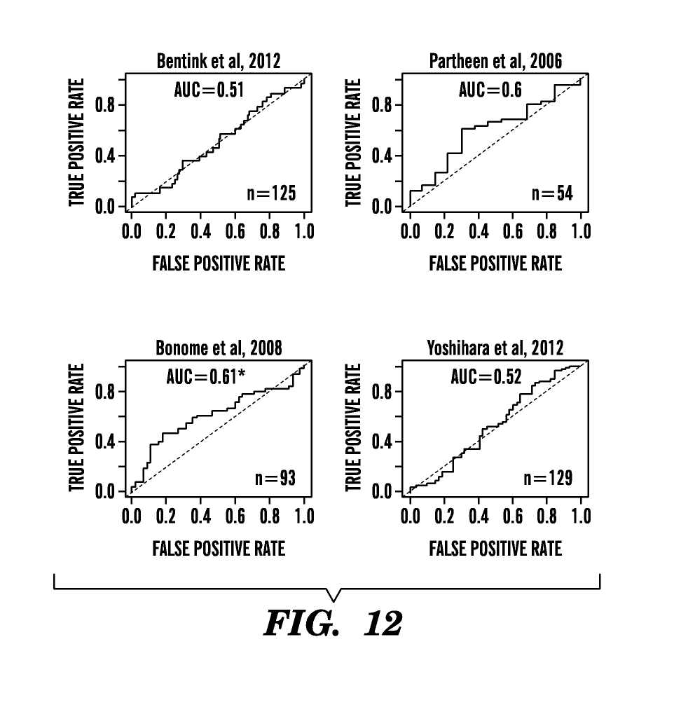

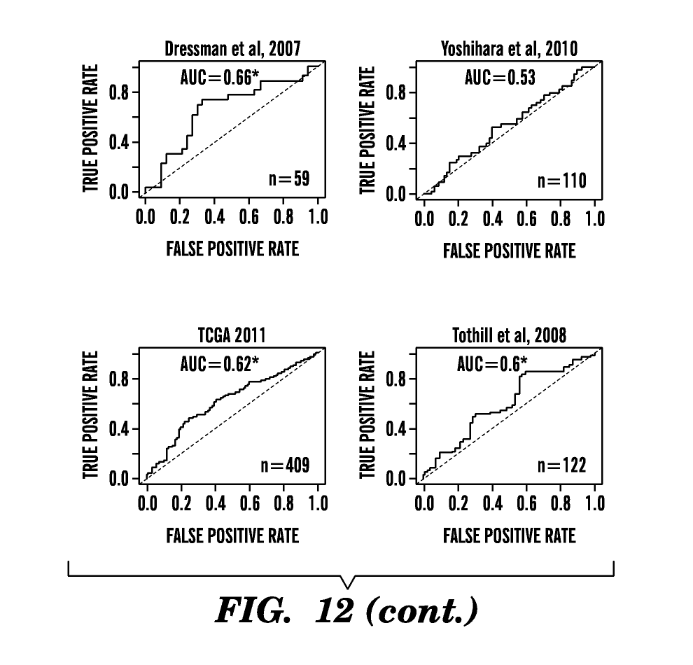

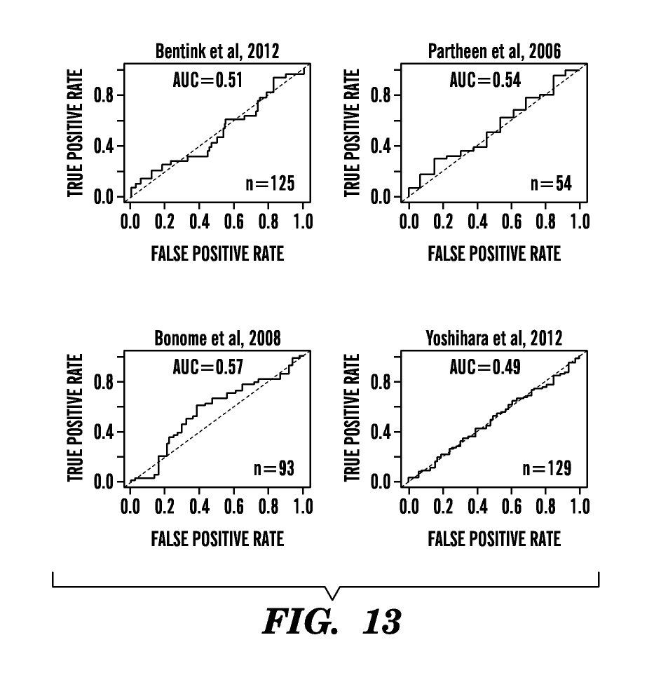

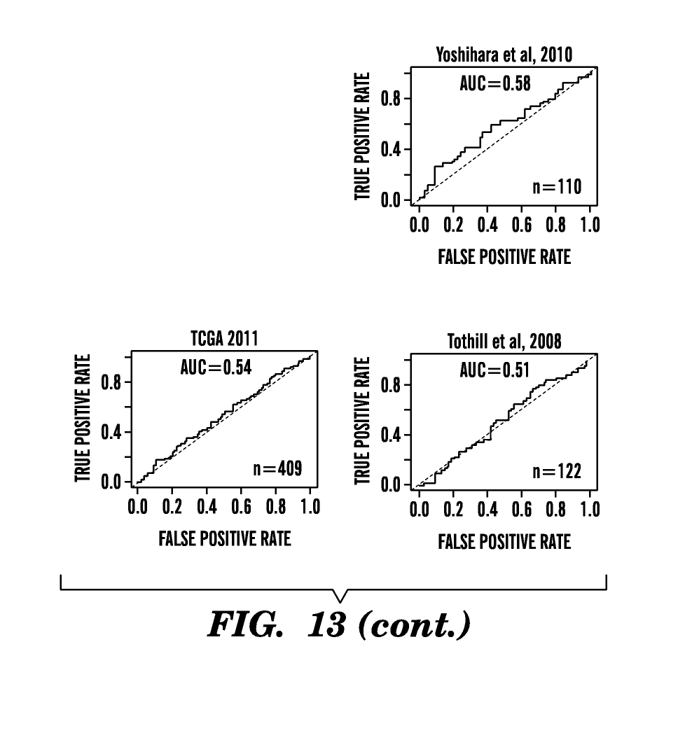

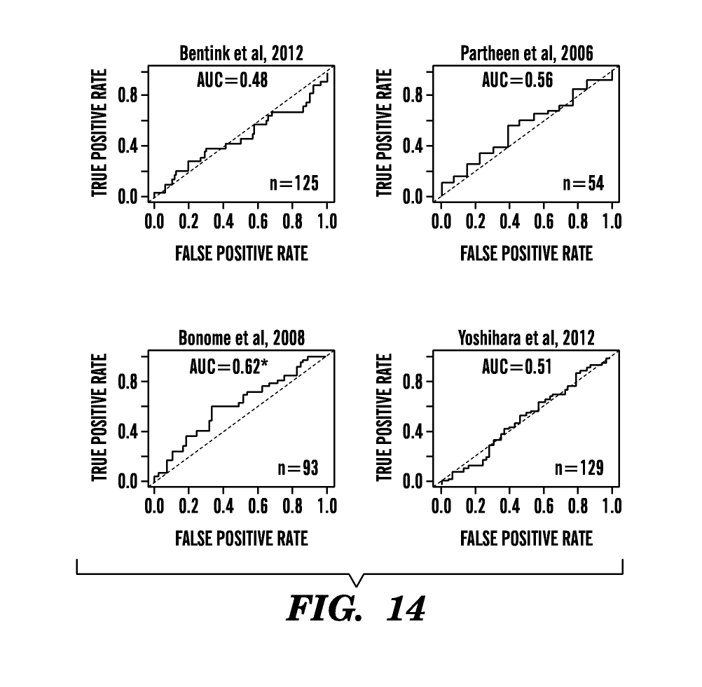

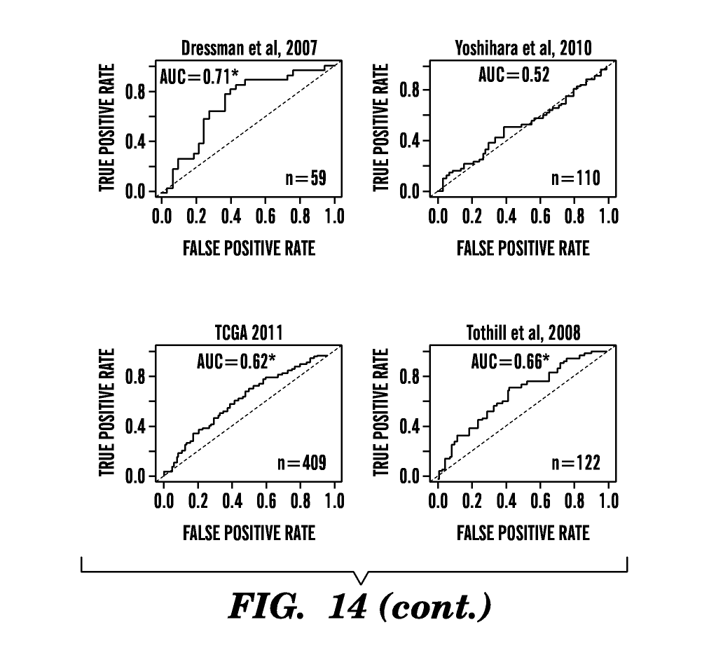

The training datasets were first tested by leave-one-dataset-out validation. Accurate prediction of debulking status proved to be difficult, with an overall AUC of 0.59 (95% CI 0.55-0.63, FIG. 10). The signature published by Berchuck et al. [21] achieved an AUC of 0.55 (95% CI 0.49-0.57, FIG. 12, Table 5). Expression of the top-ranked hit POSTN alone achieved very similar prediction accuracies as the presently described signature (FIG. 13). In the 1,248 microarray samples with debulking and survival information, high POSTN levels were only borderline prognostic of survival after adjusting for debulking status (P=0.07, unadjusted P=0.002).

Signature Reveals Pathways Contributing to Suboptimal Cytoreductive Surgery.

To explore the molecular basis underlying the debulking signature, the gene signature was analyzed to identify biological pathways relevant to suboptimal disease Application of pathway identification software (Pathway studio 7.1™, Ariadne Genomics) identified the hyperactivation of TGF-β/Smad signaling (pathway enrichment analysis, p=0.0035) and the potential activation of RTK/Ras/MAPK/Egr-1, AMPK/Egr-1 and Hedgehog/Gli signaling in suboptimally debulked tumors (FIG. 4). The resultant transcriptional network leads to up-regulation of genes that support tumor dissemination, which decrease the chance of total surgically removal, reducing the possibility of optimal debulking. Potential molecular events responsible for suboptimal surgical outcome involve migration and invasion (MMP2, TIMP3, ADAMTS1, VCL, POSTN, TGFBI, SPARC, and CYR61) [27-32], angiogenesis (EGR1, SMADs, GLIB, VCAN, POSTN, CNY61 and LOX) [33-39], metastatic colonization (POSTN, VCAN and LOX) [40-42], and the activation of tumor associated fibroblasts (TAFs, ACTA2 and FAP) which play important roles in modulating the tumor microenvironment through the secretion of growth factors and extracellular matrix remodeling to support tumor dissemination via metastasis and angiogenesis [43, 44].

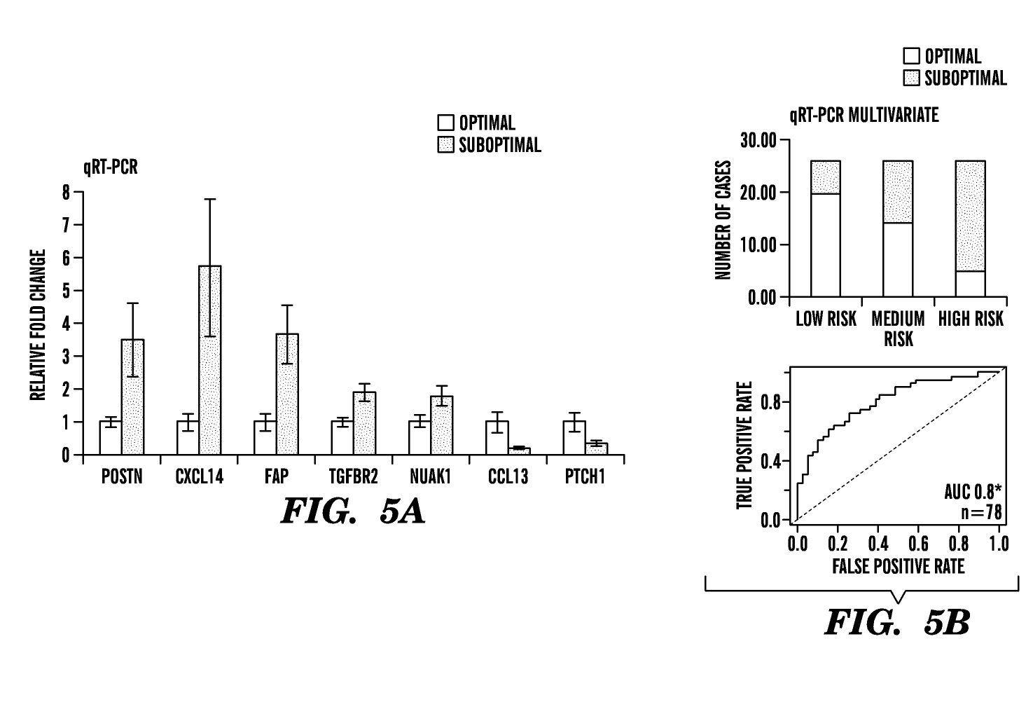

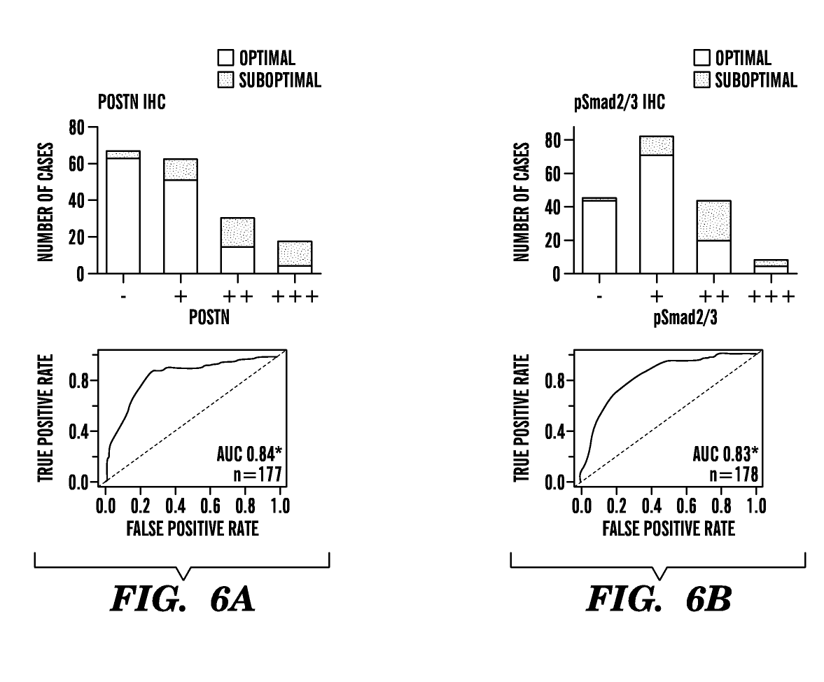

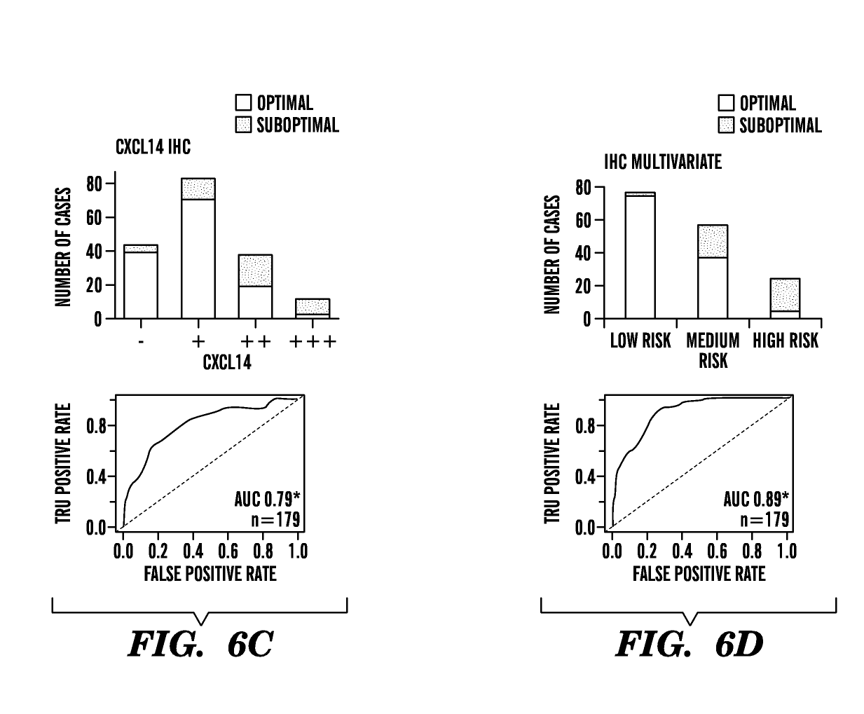

8 highly differentially expressed genes with known biological role in ovarian tumorigenesis (7 genes enriched in the pathway shown in FIG. 4) were selected and their expression level validated by qRT-PCR in an independent cohort of stage III and IV tumors consisting of 79 Bonome samples [6] excluded from the meta-analysis. Out of the 8 genes tested, 7 were significantly associated with surgery outcome: POSTN (P=0.03), CXCL14 (P=0.03), CCL13 (P=0.04), FAP (P=0.01), NUAK1 (P=0.03), PTCH1 (P=0.004), TGFBR2 (P=0.005) and TNFAIP6 (P=0.95, all Student t-test, FIG. 5A). A model using all 8 genes classified 78.8% of all samples correctly, with an AUC of 0.8 (95% CI 0.71-0.90, FIG. 5B). Using normalized microarray data for the same set of patients, however, only achieved an AUC of 0.68 through the same model, possibly owing to the higher quantitative accuracy of qRT-PCR over microarray. The AUC for POSTN qRT-PCR measured expression level alone was 0.65. Furthermore protein expression of three of these proteins POSTN, CXCL14 (signature genes) and pSmad2/3 (a surrogate marker of TGF-β pathway activation, a signature pathway) was validated by IHC in an independent cohort of 179 patients. This analysis confirmed strong association of their expression with debulking status (FIGS. 6A-6D, Table 6). The sum of IHC intensities for these three proteins provided a tool that classified 93% of samples in the high and low risk groups for suboptimal debulking correctly, with an AUC of 0.89 (95% CI 0.84-0.93, FIG. 6D).

### Discussion

Described herein is the derivation of gene expression signatures of potential clinical utility for high-grade serous ovarian cancer that predict overall patient survival in early and late-stage cases, and an additional signature for suboptimal debulking surgery. These signatures were determined using the largest gene expression meta-analysis to date for ovarian cancer, incorporating 1,622 samples. This analysis triples the sample size of the largest previous study [7]. Novel signatures were validated, shown to provide added value compared to known clinical factors, and they consistently outperformed available gene signatures [7, 16, 21].

Multiple groups have published prognostic genomic signatures for advanced stage ovarian cancer. None of these have approached clinical value for different reasons: 1.) they were generated on relatively small sample sizes [45-49]; 2.) a lack of independent validation [47, 49]; 3.) unaudited and unreliable clinical annotation [47, 50, 51]; 4.) laboratory-specific biases such as batch effects [51, 52]; and 5.) training performed on non-representative patient cohorts [2].

The comprehensive meta-analysis described herein addresses these previously missing elements. The models described herein were trained and validated on a large number of carefully curated datasets, utilizing a robust meta-analysis framework that limited the impact of laboratory or cohort-specific biases. Prediction accuracy in validation datasets was seen to increase linearly with training sample size, even up to 1,250 samples (FIG. 10). This continual increase is striking, considering the heterogeneous surgical and medical management used by the different hospitals represented in this meta-analysis. For example, large differences in the reported rate of optimal debulking of tumors to no more than 1 cm existed, likely due to differences in surgical procedures, general medical support, and pathologic review. Such heterogeneity highlights the limitations of signatures developed from any single institution, and the need for specimens from clinical trials with precisely specified inclusion criteria. However, the initial validation of these signatures across such apparently heterogeneous cohorts provides compelling evidence of their biological underpinnings.

Although the survival signature was established entirely from late-stage serous tumors, it demonstrated promise for stratifying early-stage, high-grade serous tumors. This finding may reflect the underlying biology of recurrent early stage ovarian cancer, as these tumors have gene expression profiles similar to poor-prognosis advanced stage cancers. A reliable stratification of early-stage patients could spare low-risk patients unnecessary adjuvant chemotherapy. The observed stratification of 30 TCGA samples is encouraging (FIGS. 3A-3B), in that the large majority are identified as low-risk relative to late-stage tumors, and that these risk groups show a large difference in survival rate.

Cytoreductive surgery remains an important component of treatment for women with epithelial ovarian cancer. The ability to optimally debulk patients is an important prognostic factor [6]. Whether this fact is due to (i) the smaller residual tumor mass or (ii) an intrinsic biological element of tumors, providing less aggressive and invasive tumors an advantage in surgery, is currently unresolved [22]. We present the strongest evidence to date for the existence of a biologic basis and a predictive gene signature for debulkability of ovarian tumors.

Finally, it is interesting to note that of the top 5 misclassified optimal cases (high protein expression for all three biomarkers), two had 1 cm residual disease. This suggests that the signature may be even more accurate than appreciated and its limitation is dependent in part on the clinical classification of debulking. Further, the signature provides biologic rationale and insight into ovarian cancer spread at the time of diagnosis. TGF-β pathway activation has been documented in ovarian cancer with tumors becoming resistant to the growth inhibitory effects of the ligand. Without wishing to be bound by theory, these data suggest that in a subset of tumors, the TGF-β activated pathway stimulates EMT/TAF and other biologic processes which contribute to difficulty in debulking. These pathways may provide therapeutic targets in the neoadjuvant setting, making interval debulking more effective.

### REFERENCES

1. Siegel R, Naishadham D, Jemal A. Cancer statistics, 2012. CA Cancer J Clin 2012; 62(1):10-29.
2. Swisher E M, Taniguchi T, Karlan B Y. Molecular scores to predict ovarian cancer outcomes: a worthy goal, but not ready for prime time. J Natl Cancer Inst 2012; 104(9):642-5.
3. Ganzfried B F, Riester M, Haibe-Kains B, et al. curatedOvarianData: Clinically Annotated Data for the Ovarian Cancer Transcriptome. Database 2012; accepted.
4. Bentink S, Haibe-Kains B, Risch T, et al. Angiogenic mRNA and microRNA gene expression signature predicts a novel subtype of serous ovarian cancer. PLoS ONE 2012; 7(2).
5. Bonome T, Lee J-Y, Park D-C, et al. Expression profiling of serous low malignant potential, low-grade, and high-grade tumors of the ovary. Cancer Research 2005; 65(22):10602-10612.
6. Bonome T, Levine D, Shih J, et al. A gene signature predicting for survival in suboptimally debulked patients with ovarian cancer. Cancer Research 2008; 68(13):5478-5486.
7. Cancer Genome Atlas Research Network. Integrated genomic analyses of ovarian carcinoma. Nature 2011; 474(7353):609-615.
8. Crijns A, Fehrmann R, de Jong S, et al. Survival-related profile, pathways, and transcription factors in ovarian cancer. PLoS medicine 2009; 6(2).
9. Denkert C, Budczies J, Darb-Esfahani S, et al. A prognostic gene expression index in ovarian cancer—validation across different independent data sets. The Journal of Pathology 2009; 218(2):273-280.
10. Hernandez L, Hsu S, Davidson B, et al. Activation of NF-kappaB signaling by inhibitor of NF-kappaB kinase beta increases aggressiveness of ovarian cancer. Cancer Research 2010; 70(10):4005-4014.
11. Mok S C, Bonome T, Vathipadiekal V, et al. A Gene Signature Predictive for Outcome in Advanced Ovarian Cancer Identifies a Survival Factor: Microfibril-Associated Glycoprotein 2. Cancer Cell 2009; 16(6):521-532.
12. Sabatier R, Finetti P, Bonensea J, et al. A seven-gene prognostic model for platinum-treated ovarian carcinomas. British Journal of Cancer 2011; 105(2):304-311.
13. Yoshihara K, Tajima A, Yahata T, et al. Gene expression profile for predicting survival in advanced-stage serous ovarian cancer across two independent datasets. PLoS ONE 2010; 5(8d30371b-95cc-f8dc-a30a-fc6dc17240f8).
14. Yoshihara K, Tsunoda T, Shigemizu D, et al. High-risk ovarian cancer based on 126-gene expression signature is uniquely characterized by downregulation of antigen presentation pathway. Clinical cancer research: an official journal of the American Association for Cancer Research 2012; 18(5):1374-1385.
15. Waldron L, Haibe-Kains B, Culhane A, et al. A comparative meta-analysis of prognostic gene signatures for late-stage ovarian cancer. submitted 2012.
16. Verhaak R G, Tamayo P, Yang J Y, et al. Prognostically relevant gene signatures of high-grade serous ovarian carcinoma. J Clin Invest 2012.
17. Ganzfried B F, Riester M, Haibe-Kains B, et al. curatedOvarianData: Clinically Annotated Data for the Ovarian Cancer Transcriptome. submitted 2012.
18. Partheen K, Levan K, Osterberg L, et al. Expression analysis of stage III serous ovarian adenocarcinoma distinguishes a sub-group of survivors. European journal of cancer (Oxford, England: 1990) 2006; 42(16):2846-2854.
19. Hedges L V, Vevea J L. Fixed- and Random-Effects Models in Meta-Analysis. Psychological Methods 1998; 3(4):486-504.
20. Tukey J W. Tightening the clinical trial. Control Clin Trials 1993; 14(4):266-85.
21. Berchuck A, Iversen E S, Lancaster J M, et al. Prediction of optimal versus suboptimal cytoreduction of advanced-stage serous ovarian cancer with the use of microarrays. Am J Obstet Gynecol 2004; 190(4):910-25.
22. Borley J, Wilhelm-Benartzi C, Brown R, et al. Does tumour biology determine surgical success in the treatment of epithelial ovarian cancer? A systematic literature review. Br J Cancer 2012; 107(7):1069-74.
23. Birrer M J, Johnson M E, Hao K, et al. Whole genome oligonucleotide-based array comparative genomic hybridization analysis identified fibroblast growth factor 1 as a prognostic marker for advanced-stage serous ovarian adenocarcinomas. J Clin Oncol 2007; 25(16):2281-7.
24. Gillet J-P, Calcagno A, Varma S, et al. Multidrug Resistance-Linked Gene Signature Predicts Overall Survival of Patients with Primary Ovarian Serous Carcinoma. Clinical cancer research: an official journal of the American Association for Cancer Research 2012; 18(11):3197-3206.
25. Dressman H, Berchuck A, Chan G, et al. An integrated genomic-based approach to individualized treatment of patients with advanced-stage ovarian cancer. Journal of clinical oncology: official journal of the American Society of Clinical Oncology 2007; 25(5):517-525.
26. Konstantinopoulos P A, Spentzos D, Karlan B Y, et al. Gene expression profile of BRCAness that correlates with responsiveness to chemotherapy and with outcome in patients with epithelial ovarian cancer. J Clin Oncol 2010; 28(22):3555-61.
27. Chen J, Wang M, Xi B, et al. SPARC is a key regulator of proliferation, apoptosis and invasion in human ovarian cancer. PLoS ONE 2012; 7(8):e42413.
28. Deryugina E I, Quigley J P. Matrix metalloproteinases and tumor metastasis. Cancer Metastasis Rev 2006; 25(1):9-34.
29. Dhar A, Ray A. The CCN family proteins in carcinogenesis. Exp Oncol 2010; 32(1):2-9.
30. Ricciardelli C, Frewin K M, Tan Ide A, et al. The ADAMTS1 protease gene is required for mammary tumor growth and metastasis. Am J Pathol 2011; 179(6):3075-85.
31. Soikkeli J, Podlasz P, Yin M, et al. Metastatic outgrowth encompasses COL-I, FN1, and POSTN up-regulation and assembly to fibrillar networks regulating cell adhesion, migration, and growth. Am J Pathol 2010; 177(1):387-403.
32. Ween M P, Oehler M K, Ricciardelli C. Transforming Growth Factor-Beta-Induced Protein (TGFBI)/(betaig-H3): A Matrix Protein with Dual Functions in Ovarian Cancer. Int J Mol Sci 2012; 13(8):10461-77.
33. Baker A M, Bird D, Welti J C, et al. Lysyl oxidase plays a critical role in endothelial cell stimulation to drive tumor angiogenesis. Cancer Res 2013; 73(2):583-94.
34. Carpenter R L, Lo H W. Hedgehog pathway and GLI1 isoforms in human cancer. Discov Med 2012; 13(69):105-13.
35. Chen Y, Du X Y. Functional properties and intracellular signaling of CCN1/Cyr61. J Cell Biochem 2007; 100(6):1337-45.
36. Khachigian L M. Early growth response-1: blocking angiogenesis by shooting the messenger. Cell Cycle 2004; 3(1):10-1.
37. Pardali E, ten Dijke P. Transforming growth factor-beta signaling and tumor angiogenesis. Front Biosci 2009; 14:4848-61.
38. Zheng P S, Wen J, Ang L C, et al. Versican/PG-M G3 domain promotes tumor growth and angiogenesis. FASEB J 2004; 18(6):754-6.
39. Zhu M, Fejzo M S, Anderson L, et al. Periostin promotes ovarian cancer angiogenesis and metastasis. Gynecol Oncol 2010; 119(2):337-44.
40. Erler J T, Bennewith K L, Cox T R, et al. Hypoxia-induced lysyl oxidase is a critical mediator of bone marrow cell recruitment to form the premetastatic niche. Cancer Cell 2009; 15(1):35-44.
41. Gao D, Vandat L T, Wong S, et al. Microenvironmental regulation of epithelial-mesenchymal transitions in cancer. Cancer Res 2012; 72(19):4883-9.
42. Malanchi I, Santamaria-Martinez A, Susanto E, et al. Interactions between cancer stem cells and their niche govern metastatic colonization. Nature 2012; 481(7379):85-9.
43. Ahmed F, Steele J C, Herbert J M, et al. Tumor stroma as a target in cancer. Curr Cancer Drug Targets 2008; 8(6):447-53.
44. Schauer I G, Sood A K, Mok S, et al. Cancer-associated fibroblasts and their putative role in potentiating the initiation and development of epithelial ovarian cancer. Neoplasia 2011; 13(5):393-405.
45. Dobbin K K, Zhao Y, Simon R M. How large a training set is needed to develop a classifier for microarray data? Clin Cancer Res 2008; 14(1):108-14.
46. Koscielny S. Why Most Gene Expression Signatures of Tumors Have Not Been Useful in the Clinic. Science Translational Medicine 2010; 2(14):14ps2-14ps2.
47. Micheel C M, Nass S J, Omenn G S. Evolution of Translational Omics Lessons Learned and the Path Forward. Board on Health Care Services; Board on Health Sciences Policy; Institute of Medicine. National Academies Press 2012.
48. Sabatier R, Finetti P, Cervera N, et al. Gene expression profiling and prediction of clinical outcome in ovarian cancer. Critical Reviews in Oncology/Hematology 2009; 72(2):98-109.
49. Simon R. Development and validation of therapeutically relevant multi-gene biomarker classifiers. J Natl Cancer Inst 2005; 97(12):866-7.
50. Baggerly K A, Coombes K R. Deriving Chemosensitivity from Cell Lines: Forensic Bioinformatics and Reproducible Research in High-throughput Biology. Annals of Applied Statistics 2009; 3(4):1309-1334.
51. Baggerly K A, Coombes K R, Neeley E S. Run batch effects potentially compromise the usefulness of genomic signatures for ovarian cancer. J Clin Oncol 2008; 26(7):1186-7; author reply 1187-8.
52. Leek J T, Scharpf R B, Bravo H C, et al. Tackling the widespread and critical impact of batch effects in high-throughput data. Nat Rev Genet 2010; 11(10):733-9.
53. Tothill R W, Tinker A V, George J, et al. Novel Molecular Subtypes of Serous and Endometrioid Ovarian Cancer Linked to Clinical Outcome. Clinical Cancer Research 2008; 14(16):5198-5208.

TABLE 3
Datasets used in this study.

| | | | | Number | Stage | Subopt. | Median | Median | | |
|---|---|---|---|---|---|---|---|---|---|---|
| | Accession | | | of | IV | Debulked | Survival | Follow-up | Censoring | Median |
| Dataset | ID | Ref. | Platform | samples | (%) | (%) | (Months) | (Months) | (%) | Age |
| Bentink et al, | E.MTAB.386 | [4] | Illumnia | 128¹ | 15 | 22⁴ | 39 | 53 | 43 | 66 |
| 2012 | | | HumanRef-8 | | | | | | | |
| | | | v2 | | | | | | | |
| Partheen et al, | GSE12418 | [18] | Partheen | 54¹ | 0 | 76⁵ | Short (<3 yrs) versus long term | | | 60 |
| 2006 | | | MetaData | | | | survival (>7 yrs) information only | | | |
| Crijns et al. | GSE13876 | [8] | Operon | 157¹ | N/A | N/A | 25 | 72 | 28 | 60 |
| 2009 | | | Human v3 | | | | | | | |
| Yoshihara et al., | GSE17260 | [13] | Agilent | 110¹ | 15 | 48⁴ | 53 | 47 | 58 | N/A |
| 2010 | | | G4112a | | | | | | | |
| Mok et al, 2009 | GSE18520 | [11] | Affymetrix | 53¹ | 0 | N/A | 25 | 140 | 23 | N/A |
| | | | U133 Plus 2.0 | | | | | | | |
| Konstantinopoulos | GSE19829.GPL8300 | [26] | Affymetrix | 42¹ | N/A | N/A | 45 | 50 | 45 | N/A |
| et al, 2010 | | | U95 v2 | | | | | | | |
| Bonome et al, | GSE26712 | [6] | Affymetrix | 185¹ | 19 | 51⁴ | 46 | 90 | 30 | 63 |
| 2008 | | | U133a | | | | | | | |
| Gillet et al, | GSE30009 | [24] | TaqManqRT- | 93¹ | 18 | 22⁴ | 41 | 53 | 45 | 61 |
| 2012 | | | PCR 380 | | | | | | | |
| Yoshihara et al, | GSE32062.GPL6480 | [14] | Agilent | 129¹ | 26 | 64⁴ | 59 | 55 | 53 | N/A |
| 2012 | | | G4112a | | | | | | | |
| Tothill et al, | GSE9891 | [53] | Affymetrix | 140¹ | 8 | 43⁴ | 40 | 40 | 49 | 59 |
| 2008 | | | U133 Plus 2.0 | | | | | | | |
| Dressman et al, | PMID17290060 | [25] | Affymetrix | 59¹ | 15 | 44⁴ | 42 | 94 | 39 | N/A |
| 2007⁶ | | | U133a | | | | | | | |
| TCGA 2011 | TCGA | [7] | Affymetrix | 442^{1,2} | 16 | 80⁵ | 42 | 49 | 47 | 59 |
| | | | HT U133a | | | | | | | |
| TCGA 2011 | TCGA | [7] | Affymetrix | 30³ | 0 | 23⁵ | 70 | 37 | 73 | 60.5 |
| Early Stage | | | HT U133a | | | | | | | |

N/A = Not available.
¹Only Federation of Gynecology and Obstetrics (FIGO) stage III + IV, high grade (grade 3), serous histology;
²10 samples already included in Bonome et al. [6] were removed;
³Only FIGO stage I + II, high grade, serous subtype;
⁴Suboptimal cytoreduction defined as residual tumor mass >1 cm;
⁵Suboptimalcytoreduction defined as presence of macroscopic residual tumor;
⁶Paper was retracted because of a misalignment of genomic and survival data; the corrected data were used.

TABLE 4
QRT-PCR primers

| | Forward (5′) | SEQ ID | Reverse (3′) | SEQ ID |
|---|---|---|---|---|
| Gene | primer | NO: | primer | NO: |
| POSTN | GTTAGCCTCCTG | 19 | GGCTCGGTCTTT | 20 |
| | TGGTAAAGGA | | TCAATGGG | |
| CXCL14 | ATGAAGCCAAAG | 21 | TCTCGTTCCAGG | 22 |
| | TACCCGCA | | CGTTGTAC | |
| FAP | CTCATCCACGGA | 23 | TAAGTGGTTCGT | 24 |
| | ACAGCAGA | | GGACAGGC | |
| TGFBR2 | TGTGGGTGGGCT | 25 | GCAACAGCTATT | 26 |
| | GAGAGTTA | | GGGATGGTATC | |
| NUAK1 | GGCCTGCTGTCT | 27 | ACCATGCCAGTA | 28 |
| | TGAGTACAA | | AATCCCTACA | |
| TNFAIP6 | CCAGGCTTCCCA | 29 | GCCATGGACATC | 30 |
| | AATGAGTA | | ATCGTAACT | |
| CCL13 | ACTTGTTGCTGG | 31 | GAAGGGAAGGGG | 32 |
| | TTTGGAGTTTA | | GCTTAGAG | |
| PTCH1 | AGTGGGCACAGT | 33 | CTAGGTCGCCAA | 34 |
| | TTTCATTGTA | | TGGTAACTAA | |
| GAPDH | ACCCACTCCTCC | 35 | CACCACCCTGTT | 36 |
| | ACCTTTG | | GCTGTAG | |
| GUSB | GCAGTCCAGCGT | 37 | AGGGACCATCCA | 38 |
| | AGTTGAAAA | | ATACCTGAC | |
| ACTB | CGAGGACTTTGA | 39 | GAGAAGTGGGGT | 40 |
| | TTGCACATT | | GGCTTTTAG | |

### Example 2

### Supplemental Data

Dependency of survival gene signature size on prediction performance

In the analyses described herein, the gene signature size was fixed to 200 genes. This size was motivated by the fact that this size is sufficiently small to be practically useful in a clinical test and by the performance of validated and random signatures (Waldron et al, submitted). It was shown in Waldron et al. that smaller signatures tend to be less robust than large signatures. The algorithm used herein weighs genes according their rank (see Methods). This means increasing the signature size is expected to have only limited influence on the prediction performance at some point as the weight of the genes decreases.

FIG. 7 confirms that the signature size had only modest impact on the prediction accuracy in the algorithm, as long as the signature size was larger than 100 genes. In this Fig., the prediction accuracy is reported with the C-Index metric. The C-Index is a pairwise comparison of patients, summarizing the fraction of pairs where the patient predicted to be at higher risk in fact has shorter survival. A C-Index of 0.5 would correspond to a random model, and a C-Index of 1.0 of to a perfect model. Such a perfect model would predict the correct order in which patients die. This performance measure was used instead of Hazard Ratios because it has an easy interpretation, is essentially parameter free and does not require a dichotomization of the prediction scores.

Comparison with the TCGA Signatures.

The signature described herein was compared to the TCGA signature [3]. To apply the TCGA signature across microarray platforms, the 193 probe sets in the signature were matched to 185 unique gene symbols [5] used in curated OvarianData. The reported performance of this model was reproduced in the three TCGA test datasets [2, 4, 6] to ensure the correctness of the model implementation (FIG. 8). Among the 200 genes in the present signature, 17 overlapped with the TCGA signature (P<0.001). The p-value of this overlap between the present and the TCGA signature was calculated with the hypergeometric distribution, using the number of genes common to all training datasets as background.

The TCGA project recently published an improved gene signature called CLOVAR [7]. It was possible to map 87 of the 100 genes of the signature to probesets used in curated OvarianData. 21 of these genes overlapped with the present gene signature (P<0.001).

Comparison with the CLOVAR Multivariate Model.

All datasets were classified by TCGA subtype using a single sample GSEA variant as implemented in the GSVA package. Subtype specific gene sets were first identified with the limma package in the TCGA data using the official TCGA subtype labels. Final subtype scores were obtained by subtracting the ssGSEA scores for the down-regulated gene sets from the up-regulated genes. Default gene set sizes of 200 genes were used for each subtype (100 up- and 100 down-regulated genes per subtype). Applied back to the TCGA data, this approach classified 90.5% of the samples correctly. Note that TCGA used unified expression measures obtained from multiple platforms for training, not the Affymetrix data we used in the present meta-analysis. In FIGS. 9A-9C, an association of subtype with overall survival in is shown all datasets except TCGA consistent with the report of Verhaak et al. [7]. The immunoreactive subtype had in both TCGA and the remaining datasets the best prognosis. Poor survival was in general observed for samples classified as mesenchymal.

Consistent overlaps with the Australian Ovarian Cancer Study Group (AOCS) subtypes as published by Tothill et al. [6] were tested for. All mesenchymal samples were assigned to the AOCS cluster c1, which had poor prognosis in the Tothill data (FIG. 9C). Most immunoreactive samples were assigned to the c2 cluster and c2 samples had consistent with our results better outcome in the Tothill dataset.

Verhaak et al. proposed a multi-variate model including tumor stage, debulking status, BRCA1/2 mutation status and ssGSEA scores for the immunoreactive and mesenchymal subtypes. BRCA1/2 mutation status was unavailable for the validation datasets. As the survival association of the ssGSEA scores were discovered in most of the validation datasets, a multivariate model using the CLOVAR patient stratifications in high- and low-risk (based on the median of CLOVAR risk scores in all datasets except the validation data), tumor stage, debulking status and ssGSEA scores was tested for all 4 subtypes. This model was then 5-fold cross-validated using all datasets combined. This model performed very similarity to the one proposed by using only 2 ssGSEA scores, but did not require any biased feature selection.

The correlation of the risk scores of the present meta-analysis signature and both TCGA signatures (for Verhaak et al. based on the CLOVAR signature only, not the multivariate predictions) of Tothill samples in TCGA subtypes was compared with the official Tothill et al. clusters. For example, 35 patients of the AOCS cluster c1 were classified as Mesenchymal (data not shown).

Meta-Analysis is Superior to Single Study Training.

The training sample size was positively correlated with accuracy of patient risk stratifications, up to the maximum training sample sizes of 1,250 (FIG. 10). This finding demonstrated that (i) a meta-analysis of microarray datasets even from different platforms is superior compared to single study training and (ii) the leave-one-dataset-out approach, in which training datasets were removed to obtain un-biased estimates of the final signature's HR, is not an over-optimistic estimate, because removing training datasets as expected made the signature worse.

Comparison with the Berchuck 2004 Debulking Signature.

A gene signature for suboptimal debulking surgery was generated and validated by leave-one-dataset-out cross-validation. The meta-analysis summary is shown in FIG. 13 as ROC curves.

The present results were compared to the only published model predicting debulking success [1] the inventors were aware of, with the corresponding ROC curves shown FIG. 13. Because Berchuck et al. did not provide the coefficients of their model, the average expression of genes down-regulated in suboptimal were subtracted from the average of up-regulated genes. The authors further did not specify the exact probe sets and provided Unigene or Genbank accession numbers for only a subset of genes in their signature. It was attempted to manually identify current HGNC symbols for the genes. It was possible to map 21 of the 32 genes utilized in their model to probes used in curatedOvarianData. None of these 21 genes overlapped with the present two signatures.

In Table 5, the logistic regression coefficients of the gene signature risk scores adjusted for FIGO stage (III vs. IV) are shown.

TABLE 5
Prediction of debulking status. The table lists the regression of our
leave-one-dataset-out cross-validated meta-analysis debulking gene
signature signature and the signature published by Berchuck et al. [1].
The predictions were adjusted for tumor stage. Numbers in brackets
are standard errors.

| | Meta-Analysis | Berchuck et al. 2004 |
|---|---|---|
| Intercept | −1.07* | −1.30** |
| | (0.57) | (0.59) |
| Gene Signature | 0.01*** | 0.03 |
| | (0.00) | (0.02) |
| Stage (III vs IV) | 0.47*** | 0.55*** |
| | (0.18) | (0.19) |
| AIC | 1466.98 | 1388.23 |
| BCI | 1481.98 | 1403.07 |
| Log Likelihood | −730.49 | −691.12 |
| Deviance | 1460.98 | 1382.23 |
| Num. obs. | 1097 | 1039 |

***p < 0.01,
**p < 0.05,
*p < 0.1

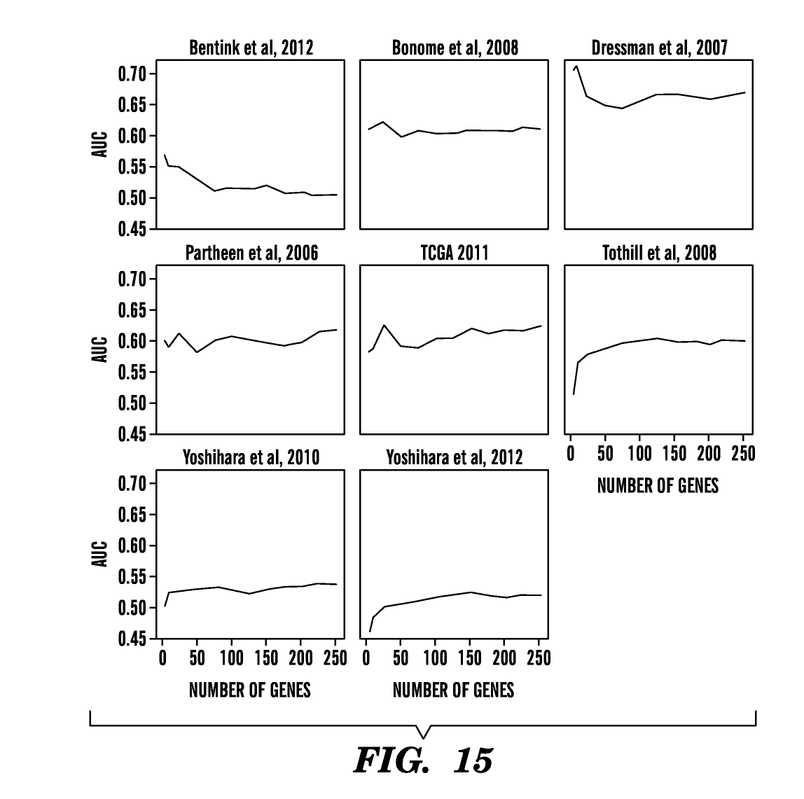

ROC curves for public microarray data of our top-ranked hit POSTN are shown in FIG. 15.

IHC and qRT-PCR Validation of Genes Associated with Surgery Out-Come.

In Table 6, it is shown that POSTN, pSmad2/3 and CXCL14 are predictors debulking surgery outcome independent of tumor grade and stage. 8 genes were further validated by qRT-PCR in 78 Bonome samples not used for training. The selection criteria were (i) high rank by fold-change and FDR and (ii) the results of our pathway analyses. For example, PTCH1 was selected because a validation would demonstrate another layout of evidence of the hyperactivation of the Hedgehog/GLI pathway in suboptimal tumors.

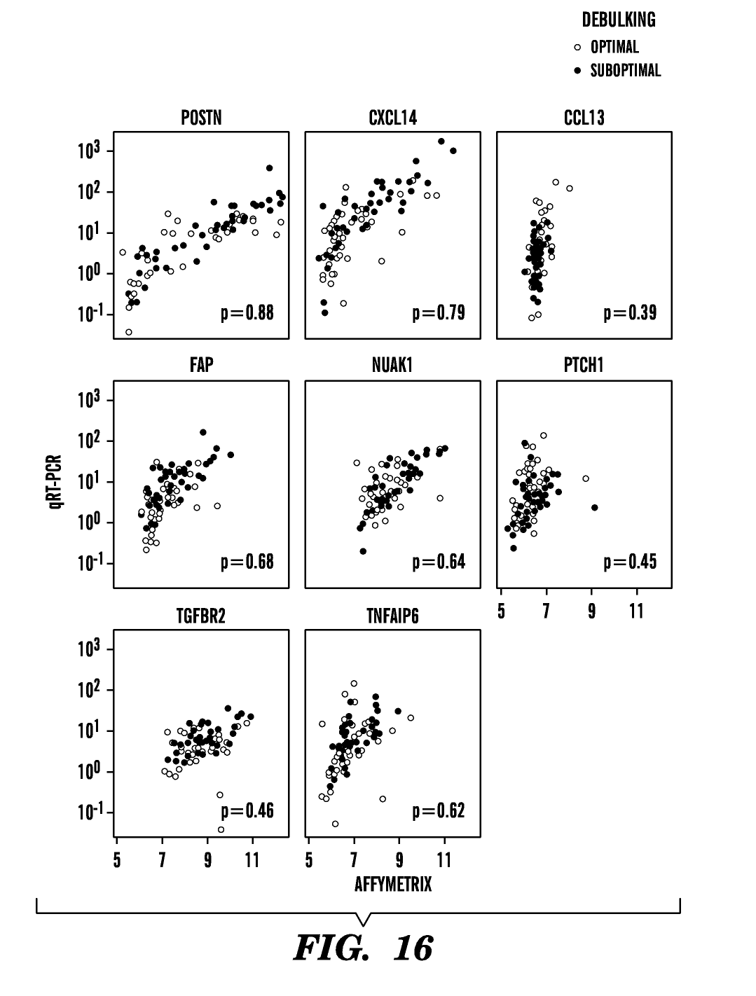

In FIG. 16, the correlation of the qRT-PCR expression values and the Affymetrix signal intensities is shown.

TABLE 6
Multivariate prediction of debulking status. Shown are multivariate models
based on IHC staining only and models adjusted (adj.) for tumor stage (III
vs. IV) and grade (2 vs. 3). Numbers in brackets are standard errors.

| | IHC | IHC adj. | POSTN adj. | pSmad2/3 adj. | CXCL14 adj. |
|---|---|---|---|---|---|
| (Intercept) | −6.35*** | −21.64*** | −15.94*** | −20.56*** | −14.39*** |
| | (1.01) | (4.72) | (4.11) | (4.21) | (3.73) |
| POSTN | 0.26*** | 0.29*** | 0.44*** | | |
| | (0.08) | (0.10) | (0.08) | | |
| pSmad23 | 0.41*** | 0.56*** | | 0.75*** | |
| | (0.12) | (0.16) | | (0.14) | |
| CXCL14 | 0.21* | 0.21 | | | 0.52*** |
| | (0.11) | (0.14) | | | (0.11) |
| Grade | | 2.32* | 1.88 | 3.10*** | 1.32 |
| | | (1.22) | (1.17) | (1.14) | (1.10) |
| Stage | | 2.25*** | 2.07*** | 1.80*** | 1.87*** |
| | | (0.61) | (0.54) | (0.51) | (0.48) |
| AIC | 129.69 | 102.43 | 120.12 | 119.14 | 135.16 |
| BCI | 142.39 | 121.17 | 132.61 | 131.66 | 147.68 |
| Log Likelihood | −60.84 | −45.21 | −56.06 | −55.57 | −63.58 |
| Deviance | 121.69 | 90.43 | 112.12 | 111.14 | 127.16 |
| Num. obs. | 177 | 168 | 168 | 169 | 169 |

***p < 0.01,
**p < 0.05,
*p < 0.1

### REFERENCES

[1] A Berchuck, E S Iversen, J M Lancaster, H K Dressman, M West, J R Nevins, and J R Marks. Prediction of optimal versus suboptimal cytoreduction of advanced-stage serous ovarian cancer with the use of microarrays. Am J Obstet Gynecol, 190(4):910-925, April 2004.
[2] T Bonome, D A Levine, J Shih, M Randonovich, C A Pise-Masison, F Bogomolniy, L Ozbun, J Brady, J C Barrett, J Boyd, and M J Birrer. A gene signature predicting for survival in suboptimally debulked patients with ovarian cancer. Cancer Res, 68(13):5478-5486, July 2008.
[3] Cancer Genome Atlas Research Network. Integrated genomic analyses of ovarian carcinoma. Nature, 474(7353):609-615, June 2011.
[4] H K Dressman, A Berchuck, G Chan, J Zhai, A Bild, R Sayer, J Cragun, J Clarke, R S Whitaker, L Li, J Gray, J Marks, G S Ginsburg, A Potti, M West, J R Nevins, and J M Lancaster. An integrated genomic-based approach to individualized treatment of patients with advanced-stage ovarian cancer. J Clin Oncol, 25(5):517-525, February 2007.
[5] R L Seal, S M Gordon, M J Lush, M W Wright, and E A Bruford. genenames.org: the hgnc resources in 2011. Nucleic Acids Res, 39(Database issue):514-519, January 2011.
[6] R W Tothill, A V Tinker, J George, R Brown, S B Fox, S Lade, D S Johnson, M K Trivett, D Etemad-moghadam, B Locandro, N Traficante, S Fereday, J A Hung, Y E Chiew, I Haviv, Australian Ovarian Cancer Study Group, D Gertig, A DeFazio, and D D Bowtell. Novel molecular subtypes of serous and endometrioid ovarian cancer linked to clinical outcome. Clin Cancer Res, 14(16):5198-5208, August 2008.
[7] R G Verhaak, P Tamayo, J Y Yang, D Hubbard, H Zhang, C J Creighton, S Fereday, M Lawrence, S L Carter, C H Mermel, A D Kostic, D Etemadmoghadam, G Saksena, K Cibulskis, S Duraisamy, K Levanon, C Sougnez, A Tsherniak, S Gomez, R Onofrio, S Gabriel, L Chin, N Zhang, P T Spellman, Y Zhang, R Akbani, K A Hoadley, A Kahn, M Kobel, D Huntsman, R A Soslow, A Defazio, M J Birrer, J W Gray, J N Weinstein, D D Bowtell, R Drapkin, J P Mesirov, G Getz, D A Levine, and M Meyerson. Prognostically relevant gene signatures of high-grade serous ovarian carcinoma. J Clin Invest, 123(1):517-525, January 2013.

1. A method of treatment comprising,
detecting, in a sample obtained from a subject in need of treatment for ovarian cancer, the level of activation of at least one pathway selected from the group consisting of:
TGF-β/Smad signaling; RTK/Ras/MAPK/Egr-1 signaling; AMPK/Egr-1 signaling; and Hedgehog/Gli signaling;
administering cytoreductive surgery to the subject if the level of activation is not increased relative to a reference level; and
not administering cytoreductive surgery to the subject if the level of activation is increased relative to a reference level.

2. A method of treatment comprising,
detecting, in a sample obtained from a subject in need of treatment for ovarian cancer, the level of expression products of at least one marker gene selected from Table 1 or Table 2;
or the level of phosphorylated SMAD2 or SMAD3;
administering cytoreductive surgery to the subject if the level of expression products selected from Table 1 or the level of phosphorylated SMAD2 or SMAD3 are not increased relative to a reference level or the level of expression products selected from Table 2 are not decreased relative to a reference level; and
not administering cytoreductive surgery to the subject if the level of expression products selected from Table 1 or the level of phosphorylated SMAD2 or SMAD3 are increased relative to a reference level or the level of expression products selected from Table 2 are decreased relative to a reference level.

3. The method of claim 2, wherein the one or more marker genes is selected from the group consisting of:
MMP2, TIMP3, ADAMTS1, VCL, TGFB1, SPARC, CYR61; EGR1, SMADs; GLIs, VCAN, CNY61, LOX, TAFs, ACTA2, POSTN, CXCL14, CCL13, FAP, NUAK1, PTCH1, TGFBR2; and TNFAIP6.

4. The method of claim 2, wherein the one or more marker genes is selected from the group consisting of:
POSTN, CXCL14, CCL13, FAP, NUAK1, PTCH1, TGFBR2; and TNFAIP6.

5. The method of claim 4, wherein the level of the expression products of POSTN, CXCL14, CCL13, FAP, NUAK1, PTCH1, TGFBR2; and TNFAIP6 is determined.

6. The method of claim 2, wherein the expression products are mRNA expression products.

7. The method of claim 2, wherein the expression products are polypeptide expression products.

8. The method of claim 2, wherein the subject has advanced stage ovarian cancer.

9. The method of claim 2, wherein the sample is a tumor cell sample.

10. The method of claim 2, wherein the subject is a human.

11-40. (canceled)


SEQUENCE LISTING (40 sequences)

SEQ ID NO: 1 - protein, 836 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 2 - protein, 111 aa, Homo sapiens
MSLLPRRAPPVSMRLLAAALLLLLLALYTARVDGSKCKCSRKGPKIRYSDVKKLEMKPKYPHCEEKMVIITTKSVSRYRGQEHCLHPKLQSTKRFIKWYNAWNEKRRVYEE

SEQ ID NO: 3 - protein, 98 aa, Homo sapiens
MKVSAVLLCLLLMTAAFNPQGLAQPDALNVPSTCCFTFSSKKISLQRLKSYVITTSRCPQKAVIFRTKLGKEICADPKEKWVQNYMKHLGRKAHTLKT

SEQ ID NO: 4 - protein, 760 aa, Homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 5 - protein, 661 aa, Homo sapiens
MEGAAAPVAGDRPDLGLGAPGSPREAVAGATAALEPRKPHGVKRHHHKHNLKHRYELQETLGKGTYGKVKRATERFSGRVVAIKSIRKDKIKDEQDMVHIRREIEIMSSLNHPHIISIYEVFENKDKIVIIMEYASKGELYDYISERRRLSERETRHFFRQIVSAVHYCHKNGVVHRDLKLENILLDDNCNIKIADFGLSNLYQKDKFLQTFCGSPLYASPEIVNGRPYRGPEVDSWALGVLLYTLVYGTMPFDGFDHKNLIRQISSGEYREPTQPSDARGLIRWMLMVNPDRRATIEDIANHWWVNWGYKSSVCDCDALHDSESPLLARIIDWHHRSTGLQADTEAKMKGLAKPTTSEVMLERQRSLKKSKKENDFAQSGQDAVPESPSKLSSKRPKGILKKRSNSEHRSHSTGFIEGVVGPALPSTFKMEQDLCRTGVLLPSSPEAEVPGKLSPKQSATMPKKGILKKTQQRESGYYSSPERSESSELLDSNDVMGSSIPSPSPPDPARVTSHSLSCRRKGILKHSSKYSAGTMDPALVSPEMPTLESLSEPGVPAEGLSRSYSRPSSVISDDSVLSSDSFDLLDLQENRPARQRIRSCVSAENFLQIQDFEGLQNRPRPQYLKRYRNRLADSSFSLLTDMDDVTQVYKQALEICSKLN

SEQ ID NO: 6 - protein, 1447 aa, Homo sapiens
MASAGNAAEPQDRGGGGSGCIGAPGRPAGGGRRRRTGGLRRAAAPDRDYLHRPSYCDAAFALEQISKGKATGRKAPLWLRAKFQRLLFKLGCYIQKNCGKFLVVGLLIFGAFAVGLKAANLETNVEELWVEVGGRVSRELNYTRQKIGEEAMFNPQLMIQTPKEEGANVLTTEALLQHLDSALQASRVHVYMYNRQWKLEHLCYKSGELITETGYMDQIIEYLYPCLIITPLDCFWEGAKLQSGTAYLLGKPPLRWTNFDPLEFLEELKKINYQVDSWEEMLNKAEVGHGYMDRPCLNPADPDCPATAPNKNSTKPLDMALVLNGGCHGLSRKYMHWQEELIVGGTVKNSTGKLVSAHALQTMFQLMTPKQMYEHFKGYEYVSHINWNEDKAAAILEAWQRTYVEVVHQSVAQNSTQKVLSFTTTTLDDILKSFSDVSVIRVASGYLLMLAYACLTMLRWDCSKSQGAVGLAGVLLVALSVAAGLGLCSLIGISFNAATTQVLPFLALGVGVDDVFLLAHAFSETGQNKRIPFEDRTGECLKRTGASVALTSISNVTAFFMAALIPIPALRAFSLQAAVVVVFNFAMVLLIFPAILSMDLYRREDRRLDIFCCFTSPCVSRVIQVEPQAYTDTHDNTRYSPPPPYSSHSFAHETQITMQSTVQLRTEYDPHTHVYYTTAEPRSEISVQPVTVTQDTLSCQSPESTSSTRDLLSQFSDSSLHCLEPPCTKWTLSSFAEKHYAPFLLKPKAKVVVIFLFLGLLGVSLYGTTRVRDGLDLTDIVPRETREYDFIAAQFKYFSFYNMYIVTQKADYPNIQHLLYDLHRSFSNVKYVMLEENKQLPKMWLHYFRDWLQGLQDAFDSDWETGKIMPNNYKNGSDDGVLAYKLLVQTGSRDKPIDISQLTKQRLVDADGIINPSAFYIYLTAWVSNDPVAYAASQANIRPHRPEWVHDKADYMPETRLRIPAAEPIEYAQFPFYLNGLRDTSDFVEAIEKVRTICSNYTSLGLSSYPNGYPFLFWEQYIGLRHWLLLFISVVLACTFLVCAVFLLNPWTAGIIVMVLALMTVELFGMMGLIGIKLSAVPVVILIASVGIGVEFTVHVALAFLTAIGDKNRRAVLALEHMFAPVLDGAVSTLLGVLMLAGSEFDFIVRYFFAVLAILTILGVLNGLVLLPVLLSFFGPYPEVSPANGLNRLPTPSPEPPPSVVRFAMPPGHTHSGSDSSDSEYSSQTTVSGLSEELRHYEAQQGAGGPAHQVIVEATENPVFAHSTVVHPESRHHPPSNPRQQPHLDSGSLPPGRQGQQPRRDPPREGLWPPPYRPRRDAFEISTEGHSGPSNRARWGPRGARSHNPRNPASTAMGSSVPGYCQPITTVTASASVTVAVHPPPVPGPGRNPRGGLCPGYPETDHGLFEDPHVPFHVRCERRDSKVEVIELQDVECEERPRGSSSN

SEQ ID NO: 7 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 8 - protein, 277 aa, Homo sapiens
MIILIYLFLLLWEDTQGWGFKDGIFHNSIWLERAAGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAKECGGVFTDPKQIFKSPGFPNEYEDNQICYWHIRLKYGQRIHLSFLDFDLEDDPGCLADYVEIYDSYDDVHGFVGRYCGDELPDDIISTGNVMTLKFLSDASVTAGGFQIKYVAMDPVSKSSQGKNTSTTSTGNKNFLAGRFSHL

SEQ ID NO: 9 - protein, 467 aa, Homo sapiens
MSSILPFTPPVVKRLLGWKKSAGGSGGAGGGEQNGQEEKWCEKAVKSLVKKLKKTGRLDELEKAITTQNCNTKCVTIPSTCSEIWGLSTPNTIDQWDTTGLYSFSEQTRSLDGRLQVSHRKGLPHVIYCRLWRWPDLHSHHELKAIENCEYAFNLKKDEVCVNPYHYQRVETPVLPPVLVPRHTEILTELPPLDDYTHSIPENTNFPAGIEPQSNYIPETPPPGYISEDGETSDQQLNQSMDTGSPAELSPTTLSPVNHSLDLQPVTYSEPAFWCSIAYYELNQRVGETFHASQPSLTVDGFTDPSNSERFCLGLLSNVNRNATVEMTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSVRCSSMS

SEQ ID NO: 10 - protein, 425 aa, Homo sapiens
MSSILPFTPPIVKRLLGWKKGEQNGQEEKWCEKAVKSLVKKLKKTGQLDELEKAITTQNVNTKCITIPRSLDGRLQVSHRKGLPHVIYCRLWRWPDLHSHHELRAMELCEFAFNMKKDEVCVNPYHYQRVETPVLPPVLVPRHTEIPAEFPPLDDYSHSIPENTNFPAGIEPQSNIPETPPPGYLSEDGETSDHQMNHSMDAGSPNLSPNPMSPAHNNLDLQPVTYCEPAFWCSISYYELNQRVGETFHASQPSMTVDGFTDPSNSERFCLGLLSNVNRNAAVELTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSIRCSSVS

SEQ ID NO: 11 - DNA, 3390 bp, Homo sapiens
agactctcaggttgatgcagtgttccctcccacaactctgacatgtatataaattctgagctctccaaagcccactgccagttctcttcggggactaactgcaacggagagactcaagatgattccctttttacccatgttttctctactattgctgcttattgttaaccctataaacgccaacaatcattatgacaagatcttggctcatagtcgtatcaggggtcgggaccaaggcccaaatgtctgtgcccttcaacagattttgggcaccaaaaagaaatacttcagcacttgtaagaactggtataaaaagtccatctgtggacagaaaacgactgtgttatatgaatgttgccctggttatatgagaatggaaggaatgaaaggctgcccagcagttttgcccattgaccatgtttatggcactctgggcatcgtgggagccaccacaacgcagcgctattctgacgcctcaaaactgagggaggagatcgagggaaagggatccttcacttactttgcaccgagtaatgaggcttgggacaacttggattctgatatccgtagaggtttggagagcaacgtgaatgttgaattactgaatgctttacatagtcacatgattaataagagaatgttgaccaaggacttaaaaaatggcatgattattccttcaatgtataacaatttggggcttttcattaaccattatcctaatggggttgtcactgttaattgtgctcgaatcatccatgggaaccagattgcaacaaatggtgttgtccatgtcattgaccgtgtgcttacacaaattggtacctcaattcaagacttcattgaagcagaagatgacctttcatcttttagagcagctgccatcacatcggacatattggaggcccttggaagagacggtcacttcacactctttgctcccaccaatgaggcttttgagaaacttccacgaggtgtcctagaaaggatcatgggagacaaagtggcttccgaagctcttatgaagtaccacatcttaaatactctccagtgttctgagtctattatgggaggagcagtctttgagacgctggaaggaaatacaattgagataggatgtgacggtgacagtataacagtaaatggaatcaaaatggtgaacaaaaaggatattgtgacaaataatggtgtgatccatttgattgatcaggtcctaattcctgattctgccaaacaagttattgagctggctggaaaacagcaaaccaccttcacggatcttgtggcccaattaggcttggcatctgctctgaggccagatggagaatacactttgctggcacctgtgaataatgcattttctgatgatactctcagcatggatcagcgcctccttaaattaattctgcagaatcacatattgaaagtaaaagttggccttaatgagctttacaacgggcaaatactggaaaccatcggaggcaaacagctcagagtcttcgtatatcgtacagctgtctgcattgaaaattcatgcatggagaaagggagtaagcaagggagaaacggtgcgattcacatattccgcgagatcatcaagccagcagagaaatccctccatgaaaagttaaaacaagataagcgctttagcaccttcctcagcctacttgaagctgcagacttgaaagagctcctgacacaacctggagactggacattatttgtgccaaccaatgatgcttttaagggaatgactagtgaagaaaaagaaattctgatacgggacaaaaatgctcttcaaaacatcattctttatcacctgacaccaggagttttcattggaaaaggatttgaacctggtgttactaacattttaaagaccacacaaggaagcaaaatctttctgaaagaagtaaatgatacacttctggtgaatgaattgaaatcaaaagaatctgacatcatgacaacaaatggtgtaattcatgttgtagataaactcctctatccagcagacacacctgttggaaatgatcaactgctggaaatacttaataaattaatcaaatacatccaaattaagtttgttcgtggtagcaccttcaaagaaatccccgtgactgtctatacaactaaaattataaccaaagttgtggaaccaaaaattaaagtgattgaaggcagtcttcagcctattatcaaaactgaaggacccacactaacaaaagtcaaaattgaaggtgaacctgaattcagactgattaaagaaggtgaaacaataactgaagtgatccatggagagccaattattaaaaaatacaccaaaatcattgatggagtgcctgtggaaataactgaaaaagagacacgagaagaacgaatcattacaggtcctgaaataaaatacactaggatttctactggaggtggagaaacagaagaaactctgaagaaattgttacaagaagaggtcaccaaggtcaccaaattcattgaaggtggtgatggtcatttatttgaagatgaagaaattaaaagactgcttcagggagacacacccgtgaggaagttgcaagccaacaaaaaagttcaaggatctagaagacgattaagggaaggtcgttctcagtgaaaatccaaaaaccagaaaaaaatgtttatacaaccctaagtcaataacctgaccttagaaaattgtgagagccaagttgacttcaggaactgaaacatcagcacaaagaagcaatcatcaaataattctgaacacaaatttaatatttttttttctgaatgagaaacatgagggaaattgtggagttagcctcctgtggtaaaggaattgaagaaaatataacaccttacaccctttttcatcttgacattaaaagttctggctaactttggaatccattagagaaaaatccttgtcaccagattcattacaattcaaatcgaagagttgtgaactgttatcccattgaaaagaccgagccttgtatgtatgttatggatacataaaatgcacgcaagccattatctctccatgggaagctaagttataaaaataggtgcttggtgtacaaaactttttatatcaaaaggctttgcacatttctatatgagtgggtttactggtaaattatgttattttttacaactaattttgtactctcagaatgtttgtcatatgcttcttgcaatgcatattttttaatctcaaacgtttcaataaaaccatttttcagatataaagagaattacttcaaattgagtaattcagaaaaactcaagatttaagttaaaaagtggtttggacttgggaacaggactttatacctcttttactgtaacaagtactcattaaaggaaattgaatgaaattaaaaaaaaaaa

SEQ ID NO: 12 - DNA, 1989 bp, Homo sapiens
aatgtgtgcgcgctgtggtatgggtgtgcaagtgtgcgaaggcggcgtgttgtgtgagcgagagggtagcggatgtgtgtgtgcgtgtgcgcgcgtggctccgggtgtgcgccgctgcgatagcgggtcctttcccggggcgggcgacgggcgggctgggaaggtctcctcccctcaccacattgagaaatctcagtgagtcaccgagtggttctgcatattaatgagctcgctcgctgcgagggcaggagcggatttaaaagaggccagggcgggcggagggaggctgtggagagagcgcggagacaagcgcagagcgcagcgcacggccacagacagccctgggcatccaccgacggcgcagccggagccagcagagccggaaggcgcgccccgggcagagaaagccgagcagagctgggtggcgtctccgggccgccgctccgacgggccagcgccctccccatgtccctgctcccacgccgcgcccctccggtcagcatgaggctcctggcggccgcgctgctcctgctgctgctggcgctgtacaccgcgcgtgtggacgggtccaaatgcaagtgctcccggaagggacccaagatccgctacagcgacgtgaagaagctggaaatgaagccaaagtacccgcactgcgaggagaagatggttatcatcaccaccaagagcgtgtccaggtaccgaggtcaggagcactgcctgcaccccaagctgcagagcaccaagcgcttcatcaagtggtacaacgcctggaacgagaagcgcagggtctacgaagaatagggtgaaaaacctcagaagggaaaactccaaaccagttgggagacttgtgcaaaggactttgcagattaaaaaaaaaaaaaaaaaaaaaaaaaaaaaaaaaaaaaaaaaaagcctttctttctcacaggcataagacacaaattatatattgttatgaagcactttttaccaacggtcagtttttacattttatagctgcgtgcgaaaggcttccagatgggagacccatctctcttgtgctccagacttcatcacaggctgctttttatcaaaaaggggaaaactcatgcctttcctttttaaaaaatgcttttttgtatttgtccatacgtcactatacatctgagctttataagcgcccgggaggaacaatgagcttggtggacacatttcattgcagtgttgctccattcctagcttgggaagcttccgcttagaggtcctggcgcctcggcacagctgccacgggctctcctgggcttatggccggtcacagcctcagtgtgactccacagtggcccctgtagccgggcaagcaggagcaggtctctctgcatctgttctctgaggaactcaagtttggttgccagaaaaatgtgcttcattcccccctggttaatttttacacaccctaggaaacatttccaagatcctgtgatggcgagacaaatgatccttaaagaaggtgtggggtctttcccaacctgaggatttctgaaaggttcacaggttcaatatttaatgcttcagaagcatgtgaggttcccaacactgtcagcaaaaaccttaggagaaaacttaaaaatatatgaatacatgcgcaatacacagctacagacacacattctgttgacaagggaaaaccttcaaagcatgtttctttccctcaccacaacagaacatgcagtactaaagcaatatatttgtgattccccatgtaattcttcaatgttaaacagtgcagtcctctttcgaaagctaagatgaccatgcgccctttcctctgtacatatacccttaagaacgccccctccacacactgccccccagtatatgccgcattgtactgctgtgttatatgctatgtacatgtcagaaaccattagcattgcatgcaggtttcatattctttctaagatggaaagtaataaaatatatttgaaatgtaccaaaaaaaaaaaaaaaaaa

SEQ ID NO: 13 - DNA, 861 bp, Homo sapiens
aaaaggccggcggaacagccagaggagcagagaggcaaagaaacattgtgaaatctccaactcttaaccttcaacatgaaagtctctgcagtgcttctgtgcctgctgctcatgacagcagctttcaacccccagggacttgctcagccagatgcactcaacgtcccatctacttgctgcttcacatttagcagtaagaagatctccttgcagaggctgaagagctatgtgatcaccaccagcaggtgtccccagaaggctgtcatcttcagaaccaaactgggcaaggagatctgtgctgacccaaaggagaagtgggtccagaattatatgaaacacctgggccggaaagctcacaccctgaagacttgaactctgctacccctactgaaatcaagctggagtacgtgaaatgacttttccattctcctctggcctcctcttctatgctttggaatacttctaccataattttcaaataggatgcattcggttttgtgattcaaaatgtactatgtgttaagtaatattggctattatttgacttgttgctggtttggagtttatttgagtattgctgatcttttctaaagcaaggccttgagcaagtaggttgctgtctctaagcccccttcccttccactatgagctgctggcagtgggtttgtattcggttcccaggggttgagagcatgcctgtgggagtcatggacatgaagggatgctgcaatgtaggaaggagagctctttgtgaatgtgaggtgttgctaaatatgttattgtggaaagatgaatgcaatagtaggactgctgacattttgcagaaaatacattttatttaaaatctcctaaaaaaaaaaa

SEQ ID NO: 14 - DNA, 2788 bp, Homo sapiens
aagaacgcccccaaaatctgtttctaattttacagaaatcttttgaaacttggcacggtattcaaaagtccgtggaaagaaaaaaaccttgtcctggcttcagcttccaactacaaagacagacttggtccttttcaacggttttcacagatccagtgacccacgctctgaagacagaattagctaactttcaaaaacatctggaaaaatgaagacttgggtaaaaatcgtatttggagttgccacctctgctgtgcttgccttattggtgatgtgcattgtcttacgcccttcaagagttcataactctgaagaaaatacaatgagagcactcacactgaaggatattttaaatggaacattttcttataaaacattttttccaaactggatttcaggacaagaatatcttcatcaatctgcagataacaatatagtactttataatattgaaacaggacaatcatataccattttgagtaatagaaccatgaaaagtgtgaatgcttcaaattacggcttatcacctgatcggcaatttgtatatctagaaagtgattattcaaagctttggagatactcttacacagcaacatattacatctatgaccttagcaatggagaatttgtaagaggaaatgagcttcctcgtccaattcagtatttatgctggtcgcctgttgggagtaaattagcatatgtctatcaaaacaatatctatttgaaacaaagaccaggagatccaccttttcaaataacatttaatggaagagaaaataaaatatttaatggaatcccagactgggtttatgaagaggaaatgcttgctacaaaatatgctctctggtggtctcctaatggaaaatttttggcatatgcggaatttaatgatacggatataccagttattgcctattcctattatggcgatgaacaatatcctagaacaataaatattccatacccaaaggctggagctaagaatcccgttgttcggatatttattatcgataccacttaccctgcgtatgtaggtccccaggaagtgcctgttccagcaatgatagcctcaagtgattattatttcagttggctcacgtgggttactgatgaacgagtatgtttgcagtggctaaaaagagtccagaatgtttcggtcctgtctatatgtgacttcagggaagactggcagacatgggattgtccaaagacccaggagcatatagaagaaagcagaactggatgggctggtggattctttgtttcaacaccagttttcagctatgatgccatttcgtactacaaaatatttagtgacaaggatggctacaaacatattcactatatcaaagacactgtggaaaatgctattcaaattacaagtggcaagtgggaggccataaatatattcagagtaacacaggattcactgttttattctagcaatgaatttgaagaataccctggaagaagaaacatctacagaattagcattggaagctatcctccaagcaagaagtgtgttacttgccatctaaggaaagaaaggtgccaatattacacagcaagtttcagcgactacgccaagtactatgcacttgtctgctacggcccaggcatccccatttccacccttcatgatggacgcactgatcaagaaattaaaatcctggaagaaaacaaggaattggaaaatgctttgaaaaatatccagctgcctaaagaggaaattaagaaacttgaagtagatgaaattactttatggtacaagatgattcttcctcctcaatttgacagatcaaagaagtatcccttgctaattcaagtgtatggtggtccctgcagtcagagtgtaaggtctgtatttgctgttaattggatatcttatcttgcaagtaaggaagggatggtcattgccttggtggatggtcgaggaacagctttccaaggtgacaaactcctctatgcagtgtatcgaaagctgggtgtttatgaagttgaagaccagattacagctgtcagaaaattcatagaaatgggtttcattgatgaaaaaagaatagccatatggggctggtcctatggaggatacgtttcatcactggcccttgcatctggaactggtcttttcaaatgtggtatagcagtggctccagtctccagctgggaatattacgcgtctgtctacacagagagattcatgggtctcccaacaaaggatgataatcttgagcactataagaattcaactgtgatggcaagagcagaatatttcagaaatgtagactatcttctcatccacggaacagcagatgataatgtgcactttcaaaactcagcacagattgctaaagctctggttaatgcacaagtggatttccaggcaatgtggtactctgaccagaaccacggcttatccggcctgtccacgaaccacttatacacccacatgacccacttcctaaagcagtgtttctctttgtcagactaaaaacgatgcagatgcaagcctgtatcagaatctgaaaaccttatataaacccctcagacagtttgcttattttattttttatgttgtaaaatgctagtataaacaaacaaattaatgttgttctaaaggctgttaaaaaaaagatgaggactcagaagttcaagctaaatattgtttacattttctggtactctgtgaaagaagagaaaagggagtcatgcattttgctttggacacagtgttttatcacctgttcatttgaagaaaaataataaagtcagaagttcaaaaaaaaaa

SEQ ID NO: 15 - DNA, 6821 bp, Homo sapiens
aatctcccaaggcctaaggaggcaagaggcctgcaaatcgcctcctgctcagcaaacgggttgctcagcaggcccggggtcctggtccaccccaggtccctggtttgcccacctccgatggcggccttcgctggcagggtgggcgcctctggggagccagctccgtcccggcgcctttagagccccatctcttccacgtccctggccttcctccccttccaggcggctgtccccgccggggtccagatggtgtcggagggccggcggttcgacggcgggcccggggttcagcctcccggcctccctccgtccctgactctcctttcttcggagagggcgcgggggccggggccaaagcgccgctcttggggttctcctggactcggagttgccccaggcgggcgcagctctgccccgcggggtgccagcctcgggcgggcaaggtccgtgagtcaccgcctgtaaccgaacaccaggcctccctgccccctcccccagctccggccgccaggctgcggcgacacctacaagaaaatgaaggggcgcccaggcccgcggcggccccggccgtatcgcgagcaggtcccggcggcccccggctcgcggcgctctttcttccccggccccggggctcggccagccgcaaccgccgccccggcgccagcaggaatccaggccgagcgaccggccccggagcccgaggcggcggagggcccgcggtagctgcgactggcgagcccgagagcgcccggggagggggcgcccggcttggaatttcccggtcccttccggcccagcgaggacaaagcactcctggccgccgccgccgccgccgccgtggcctacgccgcgccgcacaaagggcgagtcgcgacacgctcccatccccctcccagctcacggcggccccggccccgggtggctgcagggaggtgggggaagccctggctgcaccgcccctcgctccccctcccctggggccgcgcgagcgccgcccccgccccgtctgcgcgtcctcccggggaggggttggggggcgcggcgccccacataacactccccctcctgcgctgcgagccaccctctcccctccctcctgcaaacaccaccgcctcccctgccaccgccgccacctcgcccgacgctccacagctcgccgcggccggggggcggtgcgcggaccgtgcgcgccgcgggcgccagatgtgcagtccccgccgccgccagtgaccgagccgcagtccgagcggtatcgggccgcctccctgatgctgcgggggcgaccttgagcgtacagcggcttccctcggtggggaccccgacatcccagcgctgtgcccggtcttgccctctgtagcccggctcgccccgcgcttggacatggaaggggccgccgcgcctgtggcgggggaccgccccgacttggggctgggggcgccgggctctccccgagaggcggtggcgggggcgactgcagccctggagcccaggaagccgcacggggtgaagcggcatcaccacaagcacaacttgaagcaccgctacgagctgcaggagaccctgggcaaaggcacctacggcaaagtcaagcgggccaccgagaggttttctggccgagtggttgctataaaatccattcgtaaggacaaaattaaggatgaacaagacatggttcacatcagacgagagattgagatcatgtcatctctcaaccatcctcatatcatcagtatttatgaagtgtttgagaacaaagataagattgtgatcatcatggaatatgccagcaaaggggagctgtacgattacatcagtgagcggcgacgcctcagtgagagggagacccggcacttcttccggcagatcgtctctgctgtgcactattgtcacaagaacggtgtggtccaccgggacttgaagctggaaaatatactgctcgatgacaactgcaatattaagattgctgactttgggctttccaacctgtaccagaaggataagttcttacaaacgttttgtgggagtccactctatgcatctcctgagattgtcaatgggagaccttaccgagggccagaggtggacagctgggccctgggtgtgttgctttacactcttgtttatggaacaatgcccttcgatggtttcgatcacaaaaacctcattcggcaaatcagcagcggagagtaccgggagccaacacagccctcagatgctcgaggactcatacggtggatgctgatggtgaaccccgatcgccgggccactattgaggacattgccaaccactggtgggtgaactggggctataagagcagcgtgtgtgactgtgatgccctccatgactctgagtccccactcctggctcggatcattgactggcaccaccgttccacagggctgcaggctgacaccgaagccaaaatgaagggcctggccaaacccacgacctctgaggtcatgctagagcggcagcggtcgctgaagaaatccaagaaagagaatgactttgctcagtctggtcaggatgcagtgcctgaaagcccatccaagttgagttctaagaggcccaaggggatcctgaagaagcgaagcaacagcgagcatcgctctcacagcactggcttcattgaaggtgtagttggtcctgccttaccctctactttcaagatggagcaggacttgtgcaggactggcgtgctcctcccaagctcaccagaggcagaggtgccgggaaaactcagccccaagcagtcggccacgatgcccaagaaaggcatcttgaaaaagacccagcagagagaatcaggttactactcttccccagagcgcagtgagtcttcggagctgttggacagtaatgatgtgatgggcagcagcatcccctcccccagccccccggacccagccagggtaacctcccacagcctctcctgccggaggaagggcatcttgaaacacagcagcaaatactcagcgggcaccatggacccagccctggtcagccctgaaatgcccacactggaatccctgtcagagcctggtgtccctgccgagggcctctcccggagctacagccgcccttccagtgtcatcagcgatgacagcgtgctgtccagcgactcttttgacttgctggatttgcaggagaatcgccctgcccgccagcgcatccgcagctgcgtctctgcagaaaacttcctccagatccaggactttgaggggctccagaaccggccccggccccagtacctgaagcggtaccggaaccggctggcagacagcagcttctccctcctcacagacatggatgatgtgactcaggtctacaagcaagcgctggagatctgcagcaagctcaactagcattccagggcgcccaggggcgggcgggggtacgagggaggaaggggagcaagacttgggctcacaggctggttacctctttgctggctgtgacaacagactgaaaaaggattggcactgtctcacttggccaagtttgcagccttgagccaacacctaaaagggagaggtgggctcttctgccagttctgtcaattgtcagtcagaatttgggccctgtttggcatttgctttatggcacctcctagaggaccagctgtccaggggaggtggtattgaccggcactcagtgggtggagaggaagcatatgtggaaggagcatttccttagaagtcatccagatgcttctggaggaggggcaggagacacttgggctgtttgccttgggtgagcccaaagaacttgccccttttctccttgtattagcaagctaggtctggctgcgtggagctggcaagtagatttcagcaacttgagcttgagttgacgatcaattaatagtggctgccagttgtgctggcgtaagtggcccacatcatggggaaggagtgctgtgattgactagtaatggctaccacgggaaagggaaggggaagcagtagcactaatgctatgtagttgtcatctttgatctggctaggccctgggaatcgggtttagtcatcctgtgggatctatgttaactctttcatgccactggtgaggcatttgttaatttgctactcaactttgaggaaagacagggccttggtcagagagagaatgctctgaactctgctaaggacatagagtcagcccatggtgatttagctccttgctgttcacctcctctttcctgatgtctgccttgctctacagcacaacctcttgagggtggacagggagaaagatgatggtgtcagaggtcaaaactattatatatgacagggcacaagatggtctgtgatctttgcacagatgaatggaagttgatgcacaccaacaagaggcaacttgtcactttctttctcaatattaactggaatgctgcctcttgggttctcacctgcatggatgctttgagttggacgtgatactgtccatattctccagaggattacctggctgaaccattggctctgttcaccagtgacagatggtttccccatccactgagtgtagcatcctcagaggtaggcaagtttgcttctagggagttagcatgtagatgggatattgggatgaggaaaggaaaatcaggtagatggtgctttttttcccccaaatctaagtattctatgtcatggttttaaactttgccatgaactcctgggctttgggggaagagaaagttccattcatttaaatgaataaggtgttgaaagagtgcagggggttgggaggaagcatgtaagagagggaacatttccttagatgttacccagatggttctgggggagacagaaaagaggtgcggcaggactcttatcttaaaaagtaaacaaaacaaaacaaaacaaaacaaaaaactagatatgtaatttctaaacacccagatcacaatgacaagatgccactccaaccatgggacaccttcatgatactaggtttgtacttcctggtctctgggatgacttcagattctgctggccaaggcaaattgaactcagttcaagatggccaccactggtagacgtgtagatagaaaagaggactggtcttgggaacatctttggaaaaaccaacaaacaatagttctagggagatgagaaaaaaattcaccttacagtgctaagaaagtgcattagaatggaattgccctttccttaaggagacagtttgggctctccccttgccaccggctctggtgttttggcttatgcgttccttcaggttgagctgagcagtgtgttatgggaagctgctcaatttcctttcattcaattccacctccttcctgaactctaatagaggttaaaagggaaaaaaaaaattctgtagatagcaaattgtgtgtgtgggggggggtgggggtgtgggtgcatggaggacaacctgcaactctgagctccctacttcctgcctcatttcatgcagtcttttctgaacagcctatgctgctgccctgctggccccttgtgcacggcagctggccgtgtccgtagctgtcagtatgacttagatctagctcctacctactggttgatgtgttttttccttttgccaagtgattgagtctgtttagtagtttccatcattctagtctttaagtaaaaatgacactattgaggaaagtcagtctactcccttcttcctccccccaaacacgtgttctcttttgtcaggaaactcagccagtggactgtggcagagaaagtcctccactcagaggcagagactgagttaagtcataggtggccttaggcatctgcattgtttgcaggggttaagttttccttccagtgagggctggagggatgaattagctggtacctgaagccccgcttagctctgacactctgccaacatcctctgattctaggtgtggtgttgactgtcctttcaaggaaaaacttgcaatagagggaaaagccattaaagcagctccctgcttcatcattaagtcctgtcatccctaccagccaatcccagtcaaagaagttatgctttattcacttctgtggaattacaagtgagagacacttttaggacctgatggacaaagcaggagattcactgtcagctttcctggtcctctccttacttctgtgggccttgcaccgtcttagtttacacatctgccaaaggggtagaattacacttctttttacaggtaaatgtcaaggcacaatcagttttcaggaagtgcttcaagaccccaggtgaaatgaaaatgctaagtaccctctgaatggccatgcctgttaccaggtgctgcttcttcagatgatggggagcacttttcagggtgaaattcaggcgagttttgcccaggcctgctgtcttgagtacaaatgtgaatgatcgactgactgcttgttgccaaactggaaatgttctgtagggatttactggcatggtatcattcctagaagaaaaaaagagagaaacttgactgcacattaaaaaaaaaaaaaaatccacattgtgacttttatttaatttctattttttttggtaataaaaagttgacttttttatttgaatttgtcttttttatttattggtctgaaaggcatttcaaaggtattataataatatattggtgtaatttaattggtgcaacatgctttatggctcctgtcaaaattggttttcactcatttgattggtttgagcccagaacagcctacaggggaaaaacaagctggataaccacccaaagtgtttgtattttcgttggaaactgatttttgtttcattttggtttttgtttctgtttttatttttaaattaaataaattgcaatgaactg

SEQ ID NO: 16 - DNA, 7957 bp, Homo sapiens
gcgcccgccgtgtgagcagcagcagcggctggtctgtcaaccggagcccgagcccgagcagcctgcggccagcagcgtcctcgcaagccgagcgcccaggcgcgccaggagcccgcagcagcggcagcagcgcgccgggccgcccgggaagcctccgtccccgcggcggcggcggcggcggcggcaacatggcctcggctggtaacgccgccgagccccaggaccgcggcggcggcggcagcggctgtatcggtgccccgggacggccggctggaggcgggaggcgcagacggacgggggggctgcgccgtgctgccgcgccggaccgggactatctgcaccggcccagctactgcgacgccgccttcgctctggagcagatttccaaggggaaggctactggccggaaagcgccgctgtggctgagagcgaagtttcagagactcttatttaaactgggttgttacattcaaaaaaactgcggcaagttcttggttgtgggcctcctcatatttggggccttcgcggtgggattaaaagcagcgaacctcgagaccaacgtggaggagctgtgggtggaagttggaggacgagtaagtcgtgaattaaattatactcgccagaagattggagaagaggctatgtttaatcctcaactcatgatacagacccctaaagaagaaggtgctaatgtcctgaccacagaagcgctcctacaacacctggactcggcactccaggccagccgtgtccatgtatacatgtacaacaggcagtggaaattggaacatttgtgttacaaatcaggagagcttatcacagaaacaggttacatggatcagataatagaatatctttacccttgtttgattattacacctttggactgcttctgggaaggggcgaaattacagtctgggacagcatacctcctaggtaaacctcctttgcggtggacaaacttcgaccctttggaattcctggaagagttaaagaaaataaactatcaagtggacagctgggaggaaatgctgaataaggctgaggttggtcatggttacatggaccgcccctgcctcaatccggccgatccagactgccccgccacagcccccaacaaaaattcaaccaaacctcttgatatggcccttgttttgaatggtggatgtcatggcttatccagaaagtatatgcactggcaggaggagttgattgtgggtggcacagtcaagaacagcactggaaaactcgtcagcgcccatgccctgcagaccatgttccagttaatgactcccaagcaaatgtacgagcacttcaaggggtacgagtatgtctcacacatcaactggaacgaggacaaagcggcagccatcctggaggcctggcagaggacatatgtggaggtggttcatcagagtgtcgcacagaactccactcaaaaggtgctttccttcaccaccacgaccctggacgacatcctgaaatccttctctgacgtcagtgtcatccgcgtggccagcggctacttactcatgctcgcctatgcctgtctaaccatgctgcgctgggactgctccaagtcccagggtgccgtggggctggctggcgtcctgctggttgcactgtcagtggctgcaggactgggcctgtgctcattgatcggaatttcctttaacgctgcaacaactcaggttttgccatttctcgctcttggtgttggtgtggatgatgtttttcttctggcccacgccttcagtgaaacaggacagaataaaagaatcccttttgaggacaggaccggggagtgcctgaagcgcacaggagccagcgtggccctcacgtccatcagcaatgtcacagccttcttcatggccgcgttaatcccaattcccgctctgcgggcgttctccctccaggcagcggtagtagtggtgttcaattttgccatggttctgctcatttttcctgcaattctcagcatggatttatatcgacgcgaggacaggagactggatattttctgctgttttacaagcccctgcgtcagcagagtgattcaggttgaacctcaggcctacaccgacacacacgacaatacccgctacagccccccacctccctacagcagccacagctttgcccatgaaacgcagattaccatgcagtccactgtccagctccgcacggagtacgacccccacacgcacgtgtactacaccaccgctgagccgcgctccgagatctctgtgcagcccgtcaccgtgacacaggacaccctcagctgccagagcccagagagcaccagctccacaagggacctgctctcccagttctccgactccagcctccactgcctcgagcccccctgtacgaagtggacactctcatcttttgctgagaagcactatgctcctttcctcttgaaaccaaaagccaaggtagtggtgatcttcctttttctgggcttgctgggggtcagcctttatggcaccacccgagtgagagacgggctggaccttacggacattgtacctcgggaaaccagagaatatgactttattgctgcacaattcaaatacttttctttctacaacatgtatatagtcacccagaaagcagactacccgaatatccagcacttactttacgacctacacaggagtttcagtaacgtgaagtatgtcatgttggaagaaaacaaacagcttcccaaaatgtggctgcactacttcagagactggcttcagggacttcaggatgcatttgacagtgactgggaaaccgggaaaatcatgccaaacaattacaagaatggatcagacgatggagtccttgcctacaaactcctggtgcaaaccggcagccgcgataagcccatcgacatcagccagttgactaaacagcgtctggtggatgcagatggcatcattaatcccagcgctttctacatctacctgacggcttgggtcagcaacgaccccgtcgcgtatgctgcctcccaggccaacatccggccacaccgaccagaatgggtccacgacaaagccgactacatgcctgaaacaaggctgagaatcccggcagcagagcccatcgagtatgcccagttccctttctacctcaacggcttgcgggacacctcagactttgtggaggcaattgaaaaagtaaggaccatctgcagcaactatacgagcctggggctgtccagttaccccaacggctaccccttcctcttctgggagcagtacatcggcctccgccactggctgctgctgttcatcagcgtggtgttggcctgcacattcctcgtgtgcgctgtcttccttctgaacccctggacggccgggatcattgtgatggtcctggcgctgatgacggtcgagctgttcggcatgatgggcctcatcggaatcaagctcagtgccgtgcccgtggtcatcctgatcgcttctgttggcataggagtggagttcaccgttcacgttgctttggcctttctgacggccatcggcgacaagaaccgcagggctgtgcttgccctggagcacatgtttgcacccgtcctggatggcgccgtgtccactctgctgggagtgctgatgctggcgggatctgagttcgacttcattgtcaggtatttctttgctgtgctggcgatcctcaccatcctcggcgttctcaatgggctggttttgcttcccgtgcttttgtctttctttggaccatatcctgaggtgtctccagccaacggcttgaaccgcctgcccacaccctcccctgagccaccccccagcgtggtccgcttcgccatgccgcccggccacacgcacagcgggtctgattcctccgactcggagtatagttcccagacgacagtgtcaggcctcagcgaggagcttcggcactacgaggcccagcagggcgcgggaggccctgcccaccaagtgatcgtggaagccacagaaaaccccgtcttcgcccactccactgtggtccatcccgaatccaggcatcacccaccctcgaacccgagacagcagccccacctggactcagggtccctgcctcccggacggcaaggccagcagccccgcagggacccccccagagaaggcttgtggccacccccctacagaccgcgcagagacgcttttgaaatttctactgaagggcattctggccctagcaatagggcccgctggggccctcgcggggcccgttctcacaaccctcggaacccagcgtccactgccatgggcagctccgtgcccggctactgccagcccatcaccactgtgacggcttctgcctccgtgactgtcgccgtgcacccgccgcctgtccctgggcctgggcggaacccccgagggggactctgcccaggctaccctgagactgaccacggcctgtttgaggacccccacgtgcctttccacgtccggtgtgagaggagggattcgaaggtggaagtcattgagctgcaggacgtggaatgcgaggagaggccccggggaagcagctccaactgagggtgattaaaatctgaagcaaagaggccaaagattggaaaccccccacccccacctctttccagaactgcttgaagagaactggttggagttatggaaaagatgccctgtgccaggacagcagttcattgttactgtaaccgattgtattattttgttaaatatttctataaatatttaagagatgtacacatgtgtaatataggaaggaaggatgtaaagtggtatgatctggggcttctccactcctgccccagagtgtggaggccacagtggggcctctccgtatttgtgcattgggctccgtgccacaaccaagcttcattagtcttaaatttcagcatatgttgctgctgcttaaatattgtataatttacttgtataattctatgcaaatattgcttatgtaataggattattttgtaaaggtttctgtttaaaatattttaaatttgcatatcacaaccctgtggtagtatgaaatgttactgttaactttcaaacacgctatgcgtgataatttttttgtttaatgagcagatatgaagaaagcacgttaatcctggtggcttctctaggtgtcgttgtgtgcggtcctcttgtttggctgtgcgtgtgaacacgtgtgtgagttcaccatgtactgtactgtgattttttttttgtcttgttttgtttctctacactgtctgtaacctgtagtaggctctgacctagtcaggctggaagcgtcaggatatcttttcttcgtgctggtgagggctggccctaaacatccacctaatcctttcaaatcagcccggcaaaagctagactctcctcgtgtctacggcatctcttatgatcattggctgccatccaggaccccaatttgtgcttcagggggataatctccttctctcggatcattgtgatggatgctggaacctcagggtatggagctcacatcagttcatcatggtgggtgttagagaattcggtgacatgcctagtgctgagccttggctgggccatgagagtctgtatactctaaaaagcatgcagcatggtgcccctcttctgaccaacacacacacgacccctcccccaacacccccaaattcaagagtggatgtggccctgtcacaggtagaaaaacctatttagttaattctttcttggcccacagtctcccagaaatgatgttttgagtccctatagtttaaactccctctcttaaatggagcagctggttgaggctttctagatctgtttgcatcttctttaaaactaagtggtgagcatgcattgtggtgtagaggcaggcattatgtaggataagagctccggggggattcttcatgcaccagtgtttagggtacgtgcttcctaagtaaatccaaacattgtctccatcctccccgtcattagtgctctttcaatgtgatgtgggaaagcaggaggatggacacaccccactgaaagatgtaggcaggggcaggtctctcaaccaggcatatttttaaaagttgcttctgtactggttctcttcttttgctctgaggtgtgggctccctcatctcgtaaccagagaccagcacatgtcagggaagcacccagtgtcggctccccatccaaatccacaccagcaccttgttacagacaagaagtcagaggaaagggcggggtccctgcagggctgaagcctaagctactgtgaggcgctcacgagtggcagctcctgttactcccttttaaattacctgggaaatcttaacagaaaggtaatgggcccccagaaatacccacagcatagtgacctcagaccctgatactcaccacaaaacttttaagatgctgattgggagccgcttgtggctgctgggtgtgtgtgtgtgtgtgtgcgtgcgtgcgtgtgtgtgtgtctctgctggggaccctggccacccccctgctgctgtcttggtgcctgtcacccacatggtctgccatcctaacacccagctctgctcagaaaacgtcctgcgtggaggagggatgatgcagaattctgaagtcgacttccctctggctcctggcgtgccctcgctcccttcctgagcccagctcgtgttgcgccggaggctgcgcggcccctgatttctgcatggtgtagaactttctccaatagtcacattggcaaagggagaactggggtgggcggggggtggggctggcagggaattagaatttctctctctcttttaatagttttattttgtctgtcctgtttgttcatttggatgttttaatttttaaaaaaaaaaaaactttgctgatatttataattttgtatcataagaatgttttcctctacagtatttgtcatgccagtttataacaaaaaaaaatgcagggattttatttctattggaaacattacagctatgttttacttttggacagaatttttatttgtatagagtgcttactaatgttaaatagttcagagtatataacatttacattaaggactcatggtaggttttagggtaaggagtttaaaggaaataaatattcaaactgggtctcattgccaattttggtggaaatgagtttgtgtcatttcaattacaaagataaaagtatgccatataatttatttatatgaagatttatttttgtagtgtacatagtagtcatcaagtcttttgacagaagtatatttttaaagaatttatatgtgatgaatccataatgtctggaactttgctgagacatgagtgggcacagttttcattgtaaattacagcaaggaaagaaaatgtttaacagtgttaagagagtcagagcagagtggatattcatgcgattatgaagtgtttattagttaccattggcgacctagcatgcttctcatttcaaaccttggaaggtgaaaatgtacaaactctctaaataattaatgttcaaacactgatagaaattctaacatgaataaaaaataatataacttgttggttatatctttgtttgtagaatgctttttttctttaaaatacttgggagagacagttagtgttggagccatcagaaactgttgtttcatcttgctgaccttgtgacactcttcttttttgctctatctgatggagagagttttaggttttcctcctttttgttttgtataaatagtgggtataagcatagctctgttacatggatgtattacaaagtgggggctgagcttttagtgtgatcatcacccaaatagtgtacattgtacccattaattaatttcttatcacctgaccctatttttgttttctcataccaagaaacttcctaagttaaccatcaaaattagtccttctgtcatctctcttagcatacttatttcccttatgtgatgcctaataatgagggaacataaataaagccaaattcaaagataaaaaaaaaaaaaa

SEQ ID NO: 17 - DNA, 4704 bp, Homo sapiens
ggagagggagaaggctctcgggcggagagaggtcctgcccagctgttggcgaggagtttcctgtttcccccgcagcgctgagttgaagttgagtgagtcactcgcgcgcacggagcgacgacacccccgcgcgtgcacccgctcgggacaggagccggactcctgtgcagcttccctcggccgccgggggcctccccgcgcctcgccggcctccaggccccctcctggctggcgagcgggcgccacatctggcccgcacatctgcgctgccggcccggcgcggggtccggagagggcgcggcgcggaggcgcagccaggggtccgggaaggcgccgtccgctgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgttgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaatagctcttctggggcaggctgggccatgtccaaagaggctgcccctctcaccaaagaacagaggcagcaggaagctgcccctgaactgatgcttcctggaaaaccaagggggtcactcccctccctgtaagctgtggggataagcagaaacaacagcagcagggagtgggtgacatagagcattctatgcctttgacattgtcataggataagctgtgttagcacttcctcaggaaatgagattgatttttacaatagccaataacatttgcactttattaatgcctgtatataaatatgaatagctatgttttatatatatatatatatatctatatatgtctatagctctatatatatagccataccttgaaaagagacaaggaaaaacatcaaatattcccaggaaattggttttattggagaactccagaaccaagcagagaaggaagggacccatgacagcattagcatttgacaatcacacatgcagtggttctctgactgtaaaacagtgaactttgcatgaggaaagaggctccatgtctcacagccagctatgaccacattgcacttgcttttgcaaaataatcattccctgcctagcacttctcttctggccatggaactaagtacagtggcactgtttgaggaccagtgttcccggggttcctgtgtgcccttatttctcctggacttttcatttaagctccaagccccaaatctggggggctagtttagaaactctccctcaacctagtttagaaactctaccccatctttaataccttgaatgttttgaaccccactttttaccttcatgggttgcagaaaaatcagaacagatgtccccatccatgcgattgccccaccatctactaatgaaaaattgttctttttttcatctttcccctgcacttatgttactattctctgctcccagccttcatccttttctaaaaaggagcaaattctcactctaggctttatcgtgtttactttttcattacacttgacttgattttctagttttctatacaaacaccaatgggttccatctttctgggctcctgattgctcaagcacagtttggcctgatgaagaggatttcaactacacaatactatcattgtcaggactatgacctcaggcactctaaacatatgttttgtttggtcagcacagcgtttcaaaaagtgaagccactttataaatatttggagattttgcaggaaaatctggatccccaggtaaggatagcagatggttttcagttatctccagtccacgttcacaaaatgtgaaggtgtggagacacttacaaagctgcctcacttctcactgtaaacattagctctttccactgcctacctggaccccagtctaggaattaaatctgcacctaaccaaggtcccttgtaagaaatgtccattcaagcagtcattctctgggtatataatatgattttgactaccttatctggtgttaagatttgaagttggccttttattggactaaaggggaactcctttaagggtctcagttagcccaagtttcttttgcttatatgttaatagttttaccctctgcattggagagaggagtgctttactccaagaagctttcctcatggttaccgttctctccatcatgccagccttctcaacctttgcagaaattactagagaggatttgaatgtgggacacaaaggtcccatttgcagttagaaaatttgtgtccacaaggacaagaacaaagtatgagctttaaaactccataggaaacttgttaatcaacaaagaagtgttaatgctgcaagtaatctcttttttaaaactttttgaagctacttattttcagccaaataggaatattagagagggactggtagtgagaatatcagctctgtttggatggtggaaggtctcattttattgagatttttaagatacatgcaaaggtttggaaatagaacctctaggcaccctcctcagtgtgggtgggctgagagttaaagacagtgtggctgcagtagcatagaggcgcctagaaattccacttgcaccgtagggcatgctgataccatcccaatagctgttgcccattgacctctagtggtgagtttctagaatactggtccattcatgagatattcaagattcaagagtattctcacttctgggttatcagcataaactggaatgtagtgtcagaggatactgtggcttgttttgtttatgtttttttttcttattcaagaaaaaagaccaaggaataacattctgtagttcctaaaaatactgacttttttcactactatacataaagggaaagttttattcttttatggaacacttcagctgtactcatgtattaaaataggaatgtgaatgctatatactctttttatatcaaaagtctcaagcacttatttttattctatgcattgtttgtcttttacataaataaaatgtttattagattgaataaagcaaaatactcaggtgagcatcctgcctcctgttcccattcctagtagctaaa

SEQ ID NO: 18 - DNA, 1439 bp, Homo sapiens
agtcacatttcagccactgctctgagaatttgtgagcagcccctaacaggctgttacttcactacaactgacgatatgatcatcttaatttacttatttctcttgctatgggaagacactcaaggatggggattcaaggatggaatttttcataactccatatggcttgaacgagcagccggtgtgtaccacagagaagcacggtctggcaaatacaagctcacctacgcagaagctaaggcggtgtgtgaatttgaaggcggccatctcgcaacttacaagcagctagaggcagccagaaaaattggatttcatgtctgtgctgctggatggatggctaagggcagagttggataccccattgtgaagccagggcccaactgtggatttggaaaaactggcattattgattatggaatccgtctcaataggagtgaaagatgggatgcctattgctacaacccacacgcaaaggagtgtggtggcgtctttacagatccaaagcaaatttttaaatctccaggcttcccaaatgagtacgaagataaccaaatctgctactggcacattagactcaagtatggtcagcgtattcacctgagttttttagattttgaccttgaagatgacccaggttgcttggctgattatgttgaaatatatgacagttacgatgatgtccatggctttgtgggaagatactgtggagatgagcttccagatgacatcatcagtacaggaaatgtcatgaccttgaagtttctaagtgatgcttcagtgacagctggaggtttccaaatcaaatatgttgcaatggatcctgtatccaaatccagtcaaggaaaaaatacaagtactacttctactggaaataaaaactttttagctggaagatttagccacttataaaaaaaaaaaaaaggatgatcaaaacacacagtgtttatgttggaatcttttggaactcctttgatctcactgttattattaacatttatttattatttttctaaatgtgaaagcaatacataatttagggaaaattggaaaatataggaaactttaaacgagaaaatgaaacctctcataatcccactgcatagaaataacaagcgttaacattttcatatttttttctttcagtcatttttctatttgtggtatatgtatatatgtacctatatgtatttgcatttgaaattttggaatcctgctctatgtacagttttgtattatactttttaaatcttgaactttataaacattttctgaaatcattgattattctacaaaaacatgattttaaacagctgtaaaatattctatgatatgaatgttttatgcattatttaagcctgtctctattgttggaatttcaggtcattttcataaatattgttgcaataaatatccttgaacacacaaaaaaaaaaaaaaaa

SEQ ID NO: 19 - DNA, 22 bp, Artificial Sequence
  Description of Artificial Sequence Synthetic primer
gttagcctcctgtggtaaagga

SEQ ID NO: 20 - DNA, 20 bp, Artificial Sequence
  Description of Artificial Sequence Synthetic primer
ggctcggtcttttcaatggg

SEQ ID NO: 21 - DNA, 20 bp, Artificial Sequence
  Description of Artificial Sequence Synthetic primer
atgaagccaaagtacccgca

SEQ ID NO: 22 - DNA, 20 bp, Artificial Sequence
  Description of Artificial Sequence Synthetic primer
tctcgttccaggcgttgtac

SEQ ID NO: 23 - DNA, 20 bp, Artificial Sequence
  Description of Artificial Sequence Synthetic primer
ctcatccacggaacagcaga

SEQ ID NO: 24 - DNA, 20 bp, Artificial Sequence
  Description of Artificial Sequence Synthetic primer
taagtggttcgtggacaggc

SEQ ID NO: 25 - DNA, 20 bp, Artificial Sequence
  Description of Artificial Sequence Synthetic primer
tgtgggtgggctgagagtta

SEQ ID NO: 26 - DNA, 23 bp, Artificial Sequence
  Description of Artificial Sequence Synthetic primer
gcaacagctattgggatggtatc

SEQ ID NO: 27 - DNA, 21 bp, Artificial Sequence
  Description of Artificial Sequence Synthetic primer
ggcctgctgtcttgagtacaa

SEQ ID NO: 28 - DNA, 22 bp, Artificial Sequence
  Description of Artificial Sequence Synthetic primer
accatgccagtaaatccctaca

SEQ ID NO: 29 - DNA, 20 bp, Artificial Sequence
  Description of Artificial Sequence Synthetic primer
ccaggcttcccaaatgagta

SEQ ID NO: 30 - DNA, 21 bp, Artificial Sequence
  Description of Artificial Sequence Synthetic primer
gccatggacatcatcgtaact

SEQ ID NO: 31 - DNA, 23 bp, Artificial Sequence
  Description of Artificial Sequence Synthetic primer
acttgttgctggtttggagttta

SEQ ID NO: 32 - DNA, 20 bp, Artificial Sequence
  Description of Artificial Sequence Synthetic primer
gaagggaagggggcttagag

SEQ ID NO: 33 - DNA, 22 bp, Artificial Sequence
  Description of Artificial Sequence Synthetic primer
agtgggcacagttttcattgta

SEQ ID NO: 34 - DNA, 22 bp, Artificial Sequence
  Description of Artificial Sequence Synthetic primer
ctaggtcgccaatggtaactaa

SEQ ID NO: 35 - DNA, 19 bp, Artificial Sequence
  Description of Artificial Sequence Synthetic primer
acccactcctccacctttg

SEQ ID NO: 36 - DNA, 19 bp, Artificial Sequence
  Description of Artificial Sequence Synthetic primer
caccaccctgttgctgtag

SEQ ID NO: 37 - DNA, 21 bp, Artificial Sequence
  Description of Artificial Sequence Synthetic primer
gcagtccagcgtagttgaaaa

SEQ ID NO: 38 - DNA, 21 bp, Artificial Sequence
  Description of Artificial Sequence Synthetic primer
agggaccatccaatacctgac

SEQ ID NO: 39 - DNA, 21 bp, Artificial Sequence
  Description of Artificial Sequence Synthetic primer
cgaggactttgattgcacatt

SEQ ID NO: 40 - DNA, 21 bp, Artificial Sequence
  Description of Artificial Sequence Synthetic primer
gagaagtggggtggcttttag